(19) United States
(12) Patent Application Publication
(10) Pub. No.: US 2007/0275954 A1
(43) Pub. Date: Nov. 29, 2007

(54) Novel Fused Heterocycles and Uses Thereof

(75) Inventors: Gregory Basarab, Sudbury, MA (US); Charles Eyermann, Sudbury, MA (US); Bolin Geng, Andover, MA (US); Madhusudhan Gowravaram, Action, MA (US); James Loch III, Hopkinton, MA (US); Lawrence MacPherson, Marlborough, MA (US); Marshall Morningstar, Framingham, MA (US); George Mullen, Milford, MA (US); Alex Staz, Needham, MA (US); Andrew Kiely, Newton, MA (US)

Correspondence Address: ASTRAZENECA R&D BOSTON, 35 GATEHOUSE DRIVE, WALTHAM, MA, 02451-1215, US

(73) Assignee: AstraZeneca AB, SE-151 85 Sodertalje (SE)

(21) Appl. No.: 11/569,112

(22) PCT Filed: May 19, 2005
(86) PCT No.: PCT/SE05/00734

§ 371 Date: Nov. 14, 2006

Related U.S. Application Data
(60) Provisional application No. 60/572,815, filed on May 19, 2004.
(60) Provisional application No. 60/589,236, filed on Jul. 20, 2004.
(60) Provisional application No. 60/647,171, filed on Jan. 26, 2005.

(51) Int. Cl. A61K 31/5513; A61P 1/04; A61P 31/04; C07D 243/14; C07D 491/02

(52) U.S. Cl. 514/221; 540/568; 540/571

(57) ABSTRACT

This invention relates to novel compounds having the Formula (I) and to their pharmaceutical compositions and to their methods of use. These novel compounds provide a treatment or prophylaxis of H. pylori infection.

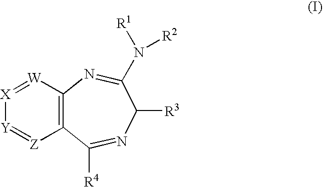

### FIELD OF THE INVENTION

The present invention relates to novel fused heterocycles, their pharmaceutical compositions and methods of use. In addition, the present invention relates to therapeutic methods for the treatment and prevention of various diseases caused by Helicobacter pylori (H. pylori) infection.

### BACKGROUND OF THE INVENTION

Helicobacter pylori (H. pylori) is a highly motile, S-shaped, microaerophilic Gram-negative bacterium that colonizes in the stomach. H. pylori infection is widespread with seroprevalence in the developed world between 30-60%. Infection with the bacterium is usually contracted during childhood and patients remain infected for life unless treated. H. pylori infection has been shown to result in the development of gastritis, peptic ulcer, and mucosa-associated lymphoid tissue (MALT) lymphoma and has been linked to gastric adenocarcinoma (Go, M. F. and D. T. Smoot, Helicobacter pylori, gastric MALT lymphoma, and adenocarcinoma of the stomach. Seminars in Gastrointestinal Disease, 2000, 11(3): p. 134-141). Eradication of H. pylori infection is currently achieved using combination therapy of antimicrobial and antisecretory agents (Malfertheiner, P., A. Leodolter, and U. Peitz, Cure of Helicobacter pylori-associated ulcer disease through eradication. Bailliere's Best Practice and Research in Clinical Gastroenterology, 2000, 14(1): p. 119-132). However, compliance to these therapies is compromised due to adverse side effects and cumbersome dosing regimens. In addition, increasing prevalence of H. pylori strains resistant to existing antimicrobial therapies threatens to limit the use of these treatments (Qureshi, W. A. and D. Y. Graham, Antibiotic-resistant H. pylori infection and its treatment. Current Pharmaceutical Design, 2000, 6(15): p. 1537-1544). Given these considerations, an ideal therapy for H. pylori infection would be a novel antimicrobial monotherapy that is selective for H. pylori eradication. The selectivity attribute is expected to aid in minimizing side effects due effects on gut flora.

H. pylori utilizes a cell wall comprised of crosslinked peptidoglycan to maintain shape and resist high osmotic pressure potentials. Peptidoglycan biosynthesis is a validated target for antimicrobial activity; cephalosphorins, penicillins and glycopeptides are antimicrobial agents, which block cell wall biosynthesis (Walsh, C., Molecular mechanisms that confer antibacterial resistance. Nature, 2000, 406: p. 775-781). Peptidoglycan biosynthesis requires the enzyme MurI, a glutamate racemase, and therefore this enzyme is essential for bacterial viability (Doublet, P., et al., The murI gene of Escherichia coli is an essential gene that encodes a glutamate racemase activity. Journal of Bacteriology, 1993, 175(10): p. 2970-9).

The present invention describes compounds, which inhibit H. pylori MurI, compositions of such compounds and methods of use. The compounds disclosed herein represent a valuable contribution to the development of selective therapies directed to diseases resulting from H. pylori infection.

### SUMMARY OF THE INVENTION

Provided herein is a compound having the structural formula (I) or a pharmaceutically acceptable salt thereof: 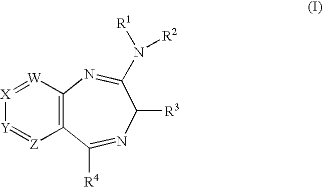
wherein:
W is N or CR^{a};
X is N or CR^{a};
Y is N or CR^{a};
Z is N or CR^{a};
provided however, only one of W, X, Y or Z is N;
R¹ and R² are, at each occurrence independently selected from H, or optionally substituted C₁₋₅alkyl, optionally substituted C₂₋₅alkenyl, optionally substituted C₂₋₅alkynyl, optionally substituted C₃₋₇cycloalkyl, optionally substituted C₃₋₇cycloalkyl-C₁₋₃allyl, optionally substituted N—C₁₋₅alkyl, optionally substituted N—C₃₋₇cycloalkyl, optionally substituted O—C₁₋₅alkyl, optionally substituted heterocyclyl, or optionally substituted phenyl; or R¹ and R² and the N to which they are attached in combination form an optionally substituted 3-7 member saturated, unsaturated or aromatic ring having 1, 2, 3 or 4 nitrogen atoms, and 0 or 1 oxygen atoms, and 0 or 1 sulfur atoms;
R³ is selected from optionally substituted C₃₋₇cycloalkyl, optionally substituted C₅₋₇cycloalkenyl, optionally substituted heterocyclyl, or optionally substituted phenyl;
R⁴ is selected from optionally substituted C₃₋₇cycloalkyl, optionally substituted C₅₋₇cycloalkenyl, optionally substituted heterocyclyl, or optionally substituted phenyl;
R^{a} is independently selected from H, cyano, halogen, nitro, C₁₋₅alkyl, C₂₋₅alkenyl, C₂₋₅ alkynyl, hydroxyl, amino, C₁₋₅alkylamino, di(C₁₋₅ alkyl)amino, C₁₋₅ alkoxyl, C₁₋₅alkylthio each substituted by zero, one or more of halogen, cyano, amino, hydroxyl, oxo carboxylate, CO₂—C₁₋₆alkyl, CONH₂, CONH—C₁₋₆alkyl, CON(C₁₋₆alkyl)-C₁₋₆alkyl, SO(C₁₋₆alkyl), SO₂(C₁₋₆alkyl), SO₂NH—C₁₋₆alkyl, SO₂NH₂, and SO₂N(C₁₋₆alkyl)-C₁₋₆alkyl.

The invention also encompasses stereoisomers, enantiomers, and pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

### DETAILED DESCRIPTION OF THE INVENTION

One embodiment of the invention is a compound having the structural formula (I) or a pharmaceutically acceptable salt thereof: 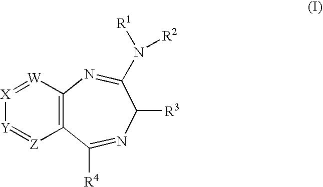
wherein:
W is N or CR^{a};
X is N or CR^{a};
Y is N or CR^{a};
Z is N or CR^{a};
provided however, only one of W, X, Y or Z is N;
R¹ and R² are, at each occurrence independently selected from H, or optionally substituted C₁₋₅alkyl, optionally substituted C₂₋₅alkenyl, optionally substituted C₂₋₅alklynyl, optionally substituted C₃₋₇cycloalkyl, optionally substituted C₃₋₇cycloalkyl-C₁₋₃alklyl, optionally substituted N—C₁₋₅alkyl, optionally substituted N—C₃₋₇cycloalkyl, optionally substituted O—C₁₋₅alkyl, optionally substituted heterocyclyl, or optionally substituted phenyl; or R¹ and R² and the N to which they are attached in combination form an optionally substituted 3-7 member saturated, unsaturated or aromatic ring having 1, 2, 3 or 4 nitrogen atoms, and 0 or 1 oxygen atoms, and 0 or 1 sulfur atoms;
R³ is selected from optionally substituted C₃₋₇cycloalkyl, optionally substituted C₅₋₇cycloalkenyl, optionally substituted heterocyclyl, or optionally substituted phenyl;
R⁴ is selected from optionally substituted C₃₋₇cycloalkyl, optionally substituted C₅₋₇cycloalkenyl, optionally substituted heterocyclyl, or optionally substituted phenyl;
R^{a} is independently selected from H, cyano, halogen, nitro, C₁₋₅alkyl, C₂₋₅alkenyl, C₂₋₅ alkynyl, hydroxyl, amino, C₁₋₅alkylamino, di(C₁₋₅ alkyl)amino, C₁₋₅ alkoxyl, C₁₋₅alkylthio each substituted by zero, one or more of halogen, cyano, amino, hydroxyl, oxo carboxylate, CO₂—C₁₋₆allyl, CONH₂, CONH—C₁₋₆alkyl, CON(C₁₋₆alkyl)-C₁₋₆alkyl, SO(C₁₋₆alkyl), SO₂(C₁₋₆alkyl), SO₂NH—C₁₋₆alkyl, SO₂NH₂, and SO₂N(C₁₋₆alkyl)-C₁₋₆alkyl.

A further embodiment is a compound of formula (I) and their pharmaceutically acceptable salts wherein:

R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl or optionally substituted O—C₁₋₅alkyl, or R¹ and R² and the N to which they are attached in combination form an optionally substituted 4-5 member saturated, unsaturated or aromatic ring having 1 additional nitrogen atoms.

A further embodiment is a compound of formula (I) and their pharmaceutically acceptable salts wherein:

R³ is selected from optionally substituted heterocyclyl, or optionally substituted phenyl.

A further embodiment is a compound of formula (I) and their pharmaceutically acceptable salts wherein:

R⁴ is selected from optionally substituted heterocyclyl, or optionally substituted phenyl, or optionally substituted C₃₋₇cycloalkyl.

A further embodiment is a compound of formula (I) and their pharmaceutically acceptable salts wherein:

R¹ is independently selected from H, NH₂, OH, halogen, C₁₋₅alkyl, cyano, nitro, trifluoromethyl, alkoxy, alkylamino.

A further embodiment is a compound of formula (I) and their pharmaceutically acceptable salts wherein:

W is N or CR^{a};

X is N or CR^{a};

Y is N or CR^{a};

Z is N or CR^{a};

provided however, only one of W, X, Y or Z is N;

R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl or optionally substituted O—C₁₋₅alkyl, or R¹ and R² and the N to which they are attached in combination form an optionally substituted 4-5 member saturated, unsaturated or aromatic ring having 1 additional nitrogen atoms;

R³ is selected from optionally substituted heterocyclyl, optionally substituted phenyl;

R⁴ is selected from optionally substituted heterocyclyl, or optionally substituted phenyl or optionally substituted C₃₋₇cycloalkyl;

R^{a} is independently selected from H, NH₂, OH, halogen, C₁₋₅allyl, cyano, nitro, trifluoromethyl, alkoxy, alkylamino.

A further embodiment is a compound of formula (I) and their pharmaceutically acceptable salts wherein:

W is N;

X is CR^{a};

Y is CR^{a};

Z is CR^{a};

R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl or optionally substituted O—C₁₋₅alkyl, or R¹ and R² and the N to which they are attached in combination form an optionally substituted 4-5 member saturated, unsaturated or aromatic ring having 1 additional nitrogen atoms;

R³ is selected from optionally substituted heterocyclyl, optionally substituted phenyl;

R⁴ is selected from optionally substituted heterocyclyl, or optionally substituted phenyl or optionally substituted C₃₋₇cycloalkyl;

R^{a} is independently selected from H, NH₂, OH, halogen, C₁₋₅alkyl, cyano, nitro, trifluoromethyl, alkoxy, alkylamino.

A further embodiment is a compound of formula (I) and their pharmaceutically acceptable salts wherein:

W is N or CR^{a};

X is N or CR^{a};

Y is N or CR^{a};

Z is N or CR^{a};

provided however, only one of W, X, Y or Z is N;

R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl, or optionally substituted O—C₁₋₅alkyl;

R³ is selected from optionally substituted heterocyclyl, optionally substituted phenyl;

R⁴ is selected from optionally substituted heterocyclyl, or optionally substituted phenyl or optionally substituted C₃₋₇cycloalkyl;

R^{a} is independently selected from H, NH₂, OH, halogen, C₁₋₅alkyl, cyano, nitro, trifluoromethyl, alkoxy, alkylamino.

A further embodiment is a compound of formula (I) and their pharmaceutically acceptable salts wherein:

W is N;

X is CR^{a};

Y is CR^{a};

Z is CR^{a};

R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl, or optionally substituted O—C₁₋₅alkyl;

R³ is selected from optionally substituted heterocyclyl, optionally substituted phenyl;

R⁴ is selected from optionally substituted heterocyclyl, or optionally substituted phenyl or optionally substituted C₃₋₇cycloalkyl;

R^{a} is independently selected from H, NH₂, OH, halogen, C₁₋₅alkyl, cyano, nitro, trifluoromethyl, alkoxy, alkylamino.

A further embodiment is a compound of formula (I) and their pharmaceutically acceptable salts wherein:

W is N or CR^{a};

X is N or CR^{a};

Y is N or CR^{a};

Z is N or CR^{a};

provided however, only one of W, X, Y or Z is N;

R¹ and R² are at each occurrence, independently selected from H, optionally substituted C₁₋₅alkyl, optionally substituted C₃₋₇cycloalkyl, optionally substituted N—C₁₋₅alkyl or optionally substituted N—C₄₋₆cycloalkyl;

R³ is selected from optionally substituted heterocyclyl, optionally substituted phenyl;

R⁴ is selected from optionally substituted heterocyclyl, or optionally substituted phenyl or optionally substituted C₃₋₇cycloalkyl;

R^{a} is independently selected from H, NH₂, OH, halogen, C₁₋₅alkyl.

A further embodiment is a compound of formula (I) and their pharmaceutically acceptable salts wherein:

W is N;

X is CR^{a};

Y is CR^{a};

Z is CR^{a};

R¹ and R² are at each occurrence, independently selected from H, optionally substituted C₁₋₅alkyl, optionally substituted C₃₋₇cycloalkyl, optionally substituted N—C₁₋₅alkyl or optionally substituted N—C₄₋₆cycloallyl;

R³ is selected from optionally substituted heterocyclyl, optionally substituted phenyl;

R⁴ is selected from optionally substituted heterocyclyl, or optionally substituted phenyl or optionally substituted C₃₋₇cycloalkyl;

R^{a} is independently selected from H, NH₂, OH, halogen, C₁₋₅alkyl.

A further embodiment is a compound of formula (I) and their pharmaceutically acceptable salts selected from:
5-(4-fluorophenyl)-N-methyl-3-phenyl-3H-1,4-benzodiazepin-2-amine;
5-(4-fluorophenyl)-N-methyl-3-thien-2-yl-3H-1,4-benzodiazepin-2-amine;
5-(4-fluorophenyl)-3-phenyl-3H-1,4-benzodiazepin-2-amine;
2-azetidin-1-yl-5-(4-fluorophenyl)-3-phenyl-3H-1,4-benzodiazepine;
N-ethyl-5-(4-fluorophenyl)-3-phenyl-3H-1,4-benzodiazepin-2-amine;
N-methyl-5-(4-methylphenyl)-3-thien-2-yl-3H-pyrido[2,3e][1,4]diazepin-2-amine;
5-(4-methoxyphenyl)-N-methyl-3-thien-2-yl-3H-pyrido[2,3-e][1,4]diazepin-2-amine;
N-cyclopropyl-5-(4-methylphenyl)-3-thien-2-yl-3H-pyrido[2,3-e][1,4]diazepin-2-amine;
N-methyl-3-pyridin-4-yl-5-thien-2-yl-3H-pyrido[2,3-e][1,4]diazepin-2-amine;
N-ethyl-3-pyridin-4-yl-5-thien-2-yl-3H-pyrido[2,3-e][1,4]diazepin-2-amine;
N-cyclopropyl-3-pyridin-4-yl-5-thien-2-yl-3H-pyrido[2,3-e][1,4]diazepin-2-amine;
3-(2-amino-1,3-thiazol-4-yl)-N-methyl-5-thien-2-yl-3H-pyrido[2,3-e][1,4]diazepin-2-amine;
5-(6-aminopyridin-3-yl)-N-methyl-3-thien-2-yl-3H-pyrido[2,3-e][1,4]diazepin-2-amine;
5-(2-aminopyridin-4-yl)-N-methyl-3-thien-2-yl-3H-pyrido[2,3-e][1,4]diazepin-2-amine;
5-(2-aminopyridin-4-yl)-N-cyclopropyl-3-thien-2-yl-3H-pyrido[2,3-e][1,4]diazepin-2-amine;
3-(3-chlorothien-2-yl)-5-(2-furyl)-N-methyl-3H-pyrido[2,3-e][1,4]diazepin-2-amine;
3-(4-bromothien-2-yl)-5-(4-fluorophenyl)-N-methyl-3H-pyrido[2,3-e][1,4]diazepin-2-amine;
3-(4-bromothien-2-yl)-N-methyl-5-thien-2-yl-3H-pyrido[2,3-e][1,4]diazepin-2-amine;
5-[2-(methylamino)-5-thien-2-yl-3H-pyrido[2,3-e][1,4]diazepin-3-yl]thiophene-3-carbonitrile;
3-(4-bromothien-2-yl)-5-(2-furyl)-N-methyl-3H-pyrido[2,3-e][1,4]diazepin-2-amine;
3-(2-furyl)-N-methyl-5-thien-2-yl-3H-pyrido[2,3-e][1,4]diazepin-2-amine;
3,5-di-2-furyl-N-methyl-3H-pyrido[2,3-e][1,4]diazepin-2-amine;
5-(2-furyl)-3-(4-iodothien-2-yl)-N-methyl-3H-pyrido[2,3-e][1,4]diazepin-2-amine;
3-{5-[5-(2-furyl)-2-(methylamino)-3H-pyrido[2,3-e][1,4]diazepin-3-yl]thien-3-yl}prop-2-yn-1-ol;
3-{4-[3-(dimethylamino)prop-1-ynyl]thien-2-yl}-5-(2-furyl)-N-methyl-3H-pyrido[2,3-e][1,4]diazepin-2-amine;
4-{5-[5-(2-furyl)-2-(methylamino)-3H-pyrido[2,3-e][1,4]diazepin-3-yl]thien-3-yl}-2-methylbut-3-yn-2-ol;
4-{5-[5-(2-furyl)-2-(methylamino)-3H-pyrido[2,3-e][1,4]diazepin-3-yl]thien-3-yl}but-3-yn-2-ol;
4-{5-[5-(2-furyl)-2-(methylamino)-3H-pyrido[2,3-e][1,4]diazepin-3-yl]thien-3-yl}but-3-yn-1-ol;
N-(3-{5-[5-(2-furyl)-2-(methylamino)-3H-pyrido[2,3-e][1,4]diazepin-3-yl]thien-3-yl}prop-2-ynyl)acetamide;
5-(4-fluorophenyl)-N,8-dimethyl-3-phenyl-3H-pyrido[2,3-e][1,4]diazepin-2-amine;
N-cyclopropyl-5-(4-fluorophenyl)-8-methyl-3-thien-2-yl-3H-pyrido[2,3-e][1,4]diazepin-2-amine;
N,8-dimethyl-5-phenyl-3-thien-2-yl-3H-pyrido[2,3-e][1,4]diazepin-2-amine;
5-(4-fluorophenyl)-N,8-dimethyl-3-thien-2-yl-3H-pyrido[2,3-e][1,4]diazepin-2-amine;
N²-cyclopropyl-5-(4-fluorophenyl)-3-thien-2-yl-3H-pyrido[2,3-e][1,4]diazepine-2,8-diamine;
5-(4-fluorophenyl)-N²-methyl-3-thien-2-yl-3H-pyrido[2,3-e][1,4]diazepine-2,8-diamine;
7-chloro-N-methyl-3,5-dithien-2-yl-3H-pyrido[2,3-e][1,4]diazepin-2-amine;
N-cyclopropyl-5-(2-furyl)-3-thien-2-yl-3H-pyrido[2,3-e][1,4]diazepin-2-amine;
N-ethyl-5-(2-furyl)-3-thien-2-yl-3H-pyrido[2,3-e][1,4]diazepin-2-amine;
5-(2-furyl)-N-prop-2-ynyl-3-thien-2-yl-3H-pyrido[2,3-e][1,4]diazepin-2-amine;
2-azetidin-1-yl-5-(2-furyl)-3-thien-2-yl-3H-pyrido[2,3-e][1,4]diazepine;
7-fluoro-5-(2-furyl)-N-methyl-3-thien-2-yl-3H-pyrido[2,3-e][1,4]diazepin-2-amine;
N-methyl-3-pyridin-3-yl-5-thien-2-yl-3H-pyrido[2,3-e][1,4]diazepin-2-amine;
5-(2-furyl)-N,8-dimethyl-3-thien-2-yl-3H-pyrido[2,3-e][1,4]diazepin-2-amine;
N,8-dimethyl-3,5-dithien-2-yl-3H-pyrido[2,3-e][1,4]diazepin-2-amine;
2-(2,2-dimethylhydrazino)-3,5-dithien-2-yl-3H-pyrido[2,3-e][1,4]diazepine;
5-(2-furyl)-N-methyl-3-(1,3-thiazol-5-yl)-3H-pyrido[2,3-e][1,4]diazepin-2-amine;
N-methyl-3-(1,3-thiazol-5-yl)-5-thien-2-yl-3H-pyrido[2,3-e][1,4]diazepin-2-amine;
N-cyclopropyl-5-(2-furyl)-3-(1,3-thiazol-5-yl)-3H-pyrido[2,3-e][1,4]diazepin-2-amine;
N-methyl-3-(1H-pyrazol-4-yl)-5-thien-2-yl-3H-pyrido[2,3-e][1,4]diazepin-2-amine;
N-methyl-3,5-di-2-thienyl-3H-pyrido[2,3-e][1,4]diazepin-2-amine;
5-cyclohexyl-N-methyl-3-thien-2-yl-3H-pyrido[2,3-e][1,4]diazepin-2-amine;
2-azetidin-1-yl-5-cyclohexyl-3-thien-2-yl-3H-pyrido[2,3-e][1,4]diazepine;
5-cyclopentyl-N-methyl-3-thien-2-yl-3H-pyrido[2,3-e][1,4]diazepin-2-amine;
N-methyl-5-phenyl-3-(2-thienyl)-3H-1,4-benzodiazepin-2-amine;
N-methyl-3,5-diphenyl-3H-1,4-benzodiazepin-2-amine;
7-chloro-N-methyl-3,5-diphenyl-3H-1,4-benzodiazepin-2-amine;
N-methyl-5-phenyl-3-thien-3-yl-3H-1,4-benzodiazepin-2-amine;
N-ethyl-5-phenyl-3-thien-2-yl-3H-1,4-benzodiazepin-2-amine;
5-phenyl-N-propyl-3-thien-2-yl-3H-1,4-benzodiazepin-2-amine;
N-isopropyl-5-phenyl-3-thien-2-yl-3H-1,4-benzodiazepin-2-amine;
N-cyclopropyl-5-phenyl-3-thien-2-yl-3H-1,4-benzodiazepin-2-amine;
N,8-dimethyl-5-phenyl-3-thien-2-yl-3H-1,4-benzodiazepin-2-amine;
N-allyl-5-phenyl-3-thien-2-yl-3H-1,4-benzodiazepin-2-amine;
5-phenyl-N-prop-2-ynyl-3-thien-2-yl-3H-1,4-benzodiazepin-2-amine;
5-phenyl-3-thien-2-yl-N-(2,2,2-trifluoroethyl)-3H-1,4-benzodiazepin-2-amine;
N,N-dimethyl-5-phenyl-3-thien-2-yl-3H-1,4-benzodiazepin-2-amine;
5-phenyl-2-pyrrolidin-1-yl-3-thien-2-yl-3H-1,4-benzodiazepine;
2-azetidin-1-yl-5-phenyl-3-thien-2-yl-3H-1,4-benzodiazepine;
2-(4,5-dihydro-1H-pyrazol-1-yl)-5-phenyl-3-thien-2-yl-3H-1,4-benzodiazepine;
2-[(5-phenyl-3-thien-2-yl-3H-1,4-benzodiazepin-2-yl)amino]ethanol;
N-(2-methoxyethyl)-5-phenyl-3-thien-2-yl-3H-1,4-benzodiazepin-2-amine;
1-(5-phenyl-3-thien-2-yl-3H-1,4-benzodiazepin-2-yl)pyrrolidin-3-ol;
1-(5-phenyl-3-thien-2-yl-3H-1,4-benzodiazepin-2-yl)azetidin-3-ol;
N-methyl-5-phenyl-3-(2-thienyl)-3H-pyrido[2,3-e][1,4]diazepin-2-amine;
N-ethyl-5-phenyl-3-thien-2-yl-3H-pyrido[2,3-e][1,4]diazepin-2-amine;
N-(2-fluoroethyl)-5-phenyl-3-thien-2-yl-3H-pyrido[2,3-e][1,4]diazepin-2-amine;
5-(4-chlorophenyl)-N-methyl-3-(2-thienyl)-3H-1,4-benzodiazepin-2-amine;
7-chloro-N-methyl-5-pyridin-3-yl-3-(2-thienyl)-3H-1,4-benzodiazepin-2-amine;
7-chloro-N-methyl-3,5-di-2-thienyl-3H-1,4-benzodiazepin-2-amine;
5-phenyl-3-(2-thienyl)-N-(2,2,2-trifluoroethyl)-3H-pyrido[2,3-e][1,4]diazepin-2-amine;
N-(2,2-difluoroethyl)-5-phenyl-3-(2-thienyl)-3H-pyrido[2,3-e][1,4]diazepin-2-amine;
1-[5-phenyl-3-(2-thienyl)-3H-pyrido[2,3-e][1,4]diazepin-2-yl]azetidin-3-ol;
1-[5-phenyl-3-(2-thienyl)-3H-pyrido[2,3-e][1,4]diazepin-2-yl]azetidin-3-amine;
N-cyclopropyl-3-(4-fluorophenyl)-5-(2-thienyl)-3H-pyrido[2,3-e][1,4]diazepin-2-amine;
N-cyclopropyl-3-(3-fluorophenyl)-5-(2-thienyl)-3H-pyrido[2,3-e][1,4]diazepin-2-amine;
N-cyclopropyl-3,5-di-2-thienyl-3H-pyrido[2,3-e][1,4]diazepin-2-amine;
N-prop-2-yn-1-yl-3,5-di-2-thienyl-3H-pyrido[2,3-e][1,4]diazepin-2-amine;
2-azetidin-1-yl-3,5-di-2-thienyl-3H-pyrido[2,3-e][1,4]diazepine;
N-ethyl-3,5-di-2-thienyl-3H-pyrido[2,3-e][1,4]diazepin-2-amine;
5-(2-furyl)-N-methyl-3-(1-methyl-1H-pyrrol-2-yl)-3H-pyrido[2,3-e][1,4]diazepin-2-amine;
N-methyl-3-(1-methyl-1H-pyrrol-2-yl)-5-(2-thienyl)-3H-pyrido[2,3-e][1,4]diazepin-2-amine;
5-(4-fluorophenyl)-N-methyl-3-thien-2-yl-3H-pyrido[2,3-e][1,4]diazepin-2-amine;
N-cyclopropyl-5-(4-fluorophenyl)-3-thien-2-yl-3H-pyrido[2,3-e][1,4]diazepin-2-amine;
N-ethyl-5-(4-fluorophenyl)-3-thien-2-yl-3H-pyrido[2,3-e][1,4]diazepin-2-amine;
5-(3-fluorophenyl)-N-methyl-3-thien-2-yl-3H-pyrido[2,3-e][1,4]diazepin-2-amine;
N-cyclopropyl-5-(3-fluorophenyl)-3-thien-2-yl-3H-pyrido[2,3-e][1,4]diazepin-2-amine;
N-ethyl-5-(3-fluorophenyl)-3-thien-2-yl-3H-pyrido[2,3-e][1,4]diazepin-2-amine;
N-methyl-5-pyridin-3-yl-3-thien-2-yl-3H-pyrido[2,3-e][1,4]diazepin-2-amine;
2-azetidin-1-yl-5-pyridin-3-yl-3-thien-2-yl-3H-pyrido[2,3-e][1,4]diazepine;
N-methyl-5-pyridin-2-yl-3-thien-2-yl-3H-pyrido[2,3-e][1,4]diazepin-2-amine;
N-methyl-3,5-di-2-thienyl-3H-1,4-benzodiazepin-2-amine;
N-methyl-3,5-di-2-thienyl-3H-pyrido[2,3-e][1,4]diazepin-2-amine;
N-methyl-5-(3-methyl-2-thienyl)-3-(2-thienyl)-3H-pyrido[2,3-e][1,4]diazepin-2-amine;
N-methyl-5-(4-methyl-1H-imidazol-5-yl)-3-(2-thienyl)-3H-pyrido[2,3-e][1,4]diazepin-2-amine;
N-methyl-5-(1,3-thiazol-2-yl)-3-(2-thienyl)-3H-pyrido[2,3-e][1,4]diazepin-2-amine;
5-(2-furyl)-N-methyl-3-(2-thienyl)-3H-pyrido[2,3-e][1,4]diazepin-2-amine;
N-methyl-5-(1,3-oxazol-2-yl)-3-thien-2-yl-3H-pyrido[2,3-e][1,4]diazepin-2-amine;
5-(1H-imidazol-4-yl)-N-methyl-3-thien-2-yl-3H-pyrido[2,3-e][1,4]diazepin-2-amine;
N-methyl-5-(1-methyl-1H-imidazol-2-yl)-3-thien-2-yl-3H-pyrido[2,3-e][1,4]diazepin-2-amine;
2-azetidin-1-yl-5-(1-methyl-1H-imidazol-2-yl)-3-thien-2-yl-3H-pyrido[2,3-e][1,4]diazepine;
N-methyl-5-(1-methyl-1H-pyrazol-3-yl)-3-thien-2-yl-3H-pyrido[2,3-e][1,4]diazepin-2-amine;
N-methyl-5-(1H-pyrrol-2-yl)-3-thien-2-yl-3H-pyrido[2,3-e][1,4]diazepin-2-amine;
N-methyl-5-(1H-pyrazol-4-yl)-3-thien-2-yl-3H-pyrido[2,3-e][1,4]diazepin-2-amine;
N-methyl-5-(1H-pyrazol-3-yl)-3-thien-2-yl-3H-pyrido[2,3-e][1,4]diazepin-2-amine;
5-(3-furyl)-N-methyl-3-thien-2-yl-3H-pyrido[2,3-e][1,4]diazepin-2-amine;
N-methyl-5-(1H-pyrrol-3-yl)-3-thien-2-yl-3H-pyrido[2,3-e][1,4]diazepin-2-amine;
2-azetidin-1-yl-5-(1H-pyrrol-3-yl)-3-thien-2-yl-3H-pyrido[2,3-e][1,4]diazepine;
N-cyclopropyl-5-(1H-pyrrol-3-yl)-3-thien-2-yl-3H-pyrido[2,3-e][1,4]diazepin-2-amine;
N-methyl-5-(1-methyl-1H-pyrazol-4-yl)-3-thien-2-yl-3H-pyrido[2,3-e][1,4]diazepin-2-amine;
N-methyl-5-(1-methyl-1H-pyrrol-3-yl)-3-thien-2-yl-3H-pyrido[2,3-e][1,4]diazepin-2-amine;
N-methyl-5-(1-methyl-1H-pyrrol-2-yl)-3-thien-2-yl-3H-pyrido[2,3-e][1,4]diazepin-2-amine;
5-isothiazol-5-yl-N-methyl-3-thien-2-yl-3H-pyrido[2,3-e][1,4]diazepin-2-amine;
N-methyl-3-thien-2-yl-5-thien-3-yl-3H-pyrido[2,3-e][1,4]diazepin-2-amine;
2-azetidin-1-yl-3-thien-2-yl-5-thien-3-yl-3H-pyrido[2,3-e][1,4]diazepine;
N-cyclopropyl-3-thien-2-yl-5-thien-3-yl-3H-pyrido[2,3-e][1,4]diazepin-2-amine;
N-methyl-3-(5-methylthien-2-yl)-5-thien-2-yl-3H-pyrido[2,3-e][1,4]diazepin-2-amine;
3-(5-chlorothien-2-yl)-N-methyl-5-thien-2-yl-3H-pyrido[2,3-e][1,4]diazepin-2-amine;
3-(5-chlorothien-2-yl)-N-cyclopropyl-5-thien-2-yl-3H-pyrido[2,3-e][1,4]diazepin-2-amine;
N-methyl-3-(3-methylthien-2-yl)-5-thien-2-yl-3H-pyrido[2,3-e][1,4]diazepin-2-amine;
N-cyclopropyl-3-(3-methylthien-2-yl)-5-thien-2-yl-3H-pyrido[2,3-e][1,4]diazepin-2-amine;
5-(2-furyl)-N-methyl-3-(3-methylthien-2-yl)-3H-pyrido[2,3-e][1,4]diazepin-2-amine;
N-methyl-3-(1,3-thiazol-2-yl)-5-thien-2-yl-3H-pyrido[2,3-e][1,4]diazepin-2-amine;
5-(2-furyl)-N-methyl-3-(1,3-thiazol-2-yl)-3H-pyrido[2,3-e][1,4]diazepin-2-amine;
3-cyclopentyl-5-(2-furyl)-N-methyl-3H-pyrido[2,3-e][1,4]diazepin-2-amine;
3-(4-chloro-2-thienyl)-N-methyl-5-(2-thienyl)-3H-pyrido[2,3-e][1,4]diazepin-2-amine;
3-(3-chloro-2-thienyl)-N-methyl-5-(2-thienyl)-3H-pyrido[2,3-e][1,4]diazepin-2-amine.

Further provided herein are compounds of formula (I) and their pharmaceutically acceptable salts for use as a medicament.

Further provided herein are compounds of formula (I) and their pharmaceutically acceptable salts, for use in the manufacture of a medicament for the treatment or prophylaxis of disorders associated with H. pylori infection.

Further provided herein is the use of a compound of formula (I) or their pharmaceutically acceptable salts for the treatment of a disorder such as gastritis, mucosa-associated lymphoid tissue lymphoma, gastric adenocarcinoma, adenocarcinoma of the stomach, or ulcer diseases including but not limited to pepetic ulcer.

Further provided herein is a method for the treatment of infections associated with H. pylori comprising administering to a host in need of such treatment a therapeutically effective amount of a compound of formula (I) or a pharmaceutically acceptable salt.

Further provided herein is a method for the prophylaxis treatment of infections associated with H. pylori comprising administering to a host in need of such treatment a therapeutically effective amount of a compound of formula (I) or a pharmaceutically acceptable salt.

Further provided herein is a method for the prophylaxis treatment of infections associated

Further provided is a pharmaceutical composition comprising a compound of formula (I) or a pharmaceutically acceptable salt, in combination with a pharmaceutically acceptable carrier, diluent or excipent.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the formula I.

Compounds of the invention are useful in disease states where infection is present or implicated. This may involve the use of isotopically labelled versions of the compounds of the invention in diagnostic techniques and imaging applications such as positron emission tomography (PET).

Definitions

The definitions set forth in this section are intended to clarify terms used throughout this application. The term “herein” means the entire application.

As used in this application, the term “optionally substituted,” as used herein, means that substitution is optional and therefore it is possible for the designated atom to be unsubstituted. In the event a substitution is desired then such substitution means that any number of hydrogens on the designated atom is replaced with a selection from the indicated group, provided that the normal valency of the designated atom is not exceeded, and that the substitution results in a stable compound. For example when a substituent is keto (i.e., ═O), then 2 hydrogens on the atom are replaced. Other such examples include: amino, cyano, halogen, hydroxy, keto, oxo, nitro, trifluoromethyl, C₁₋₁₀alkyl, C₁₋₁₀alkyl-carbonyl, C₁₋₁₀alkyl-carbonyl oxime, carboxaldehyde, carboxaldehyde oxime, C₂₋₁₀alkenyl, C₂₋₁₀ alkynyl, C₁₋₁₀alkylamino, C₁₋₁₀ alkoxyl, C₁₋₁₀alkylthio, carboxamide, N—C₁₋₇alkyl carboxamide, N,N-di(C₁₋₇alkyl)carboxamide, carboxylate, C₁₋₇alkyl carboxylate, C₃₋₆cycloalkyl, O—CO—C₁₋₆alkyl, NHCHO, N(C₁₋₆alkyl)CHO, NH—CO—C₁₋₆alkyl, NH—CO-amino, N(C₁₋₆alkyl)-CO—C₁₋₆alkyl, NH—CO₂C₁₋₆alkyl, NHCHO, NH—CO—C₁₋₆alkyl, NH—CO-amino, NH—CO—NH(C₁₋₆alkyl), N(C₁₋₆alkyl)-CO—C₁₋₆alkyl, SO(C₁₋₆alkyl), SO₂(C₁₋₆alkyl), SO₂NH—C₁₋₆alkyl, SO₂NH₂, and SO₂N(C₁₋₆alkyl)-C₁₋₆alkyl, CO₂H, CO₂C₁₋₆alkyl, mercapto, aryl or heterocyclyl each substituted with zero, one or more groups selected from hydroxyl, C₁₋₄alkoxyl, amino, cyano, oxo, halogen, C₂₋₄alkylcarbonyl, C₂₋₄alkoxylcarbonyl, C₂₋₄alkylaminocarbonyl, aminocarbonyl, and heterocyclyl;

When any variable (e.g., R¹, R⁷, R^{a}, R^{e} etc.) occurs more than one time in any constituent or formula for a compound, its definition at each occurrence is independent of its definition at every other occurrence. Thus, for example, if a group is shown to be substituted with 0-3 R¹, then said group may optionally be substituted with 0,1, 2 or 3 R¹ groups and R^{e} at each occurrence is selected independently from the definition of R^{e}. Also, combinations of substituents and/or variables are permissible only if such combinations result in stable compounds.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein “acyl” refers to radicals of the of the general formula —C(═O)—R, wherein R is hydrogen, hydrocarbyl radical, amino or alkoxy. Acyl groups include, for example, acetyl, propionyl, benzoyl, phenyl acetyl, carboethoxy, and dimethylcarbamoyl.

As used herein “aromatic” refers to hydrocarbyl radicals having one or more polyunsaturated carbon rings having aromatic character, (e.g., 4n+2 delocalized electrons) and comprising up to about 14 carbon atoms.

As used herein, “alkyl” or “alkylene” is intended to include both branched and straight-chain saturated aliphatic hydrocarbon groups having the specified number of carbon atoms. For example, “C₁₋₆ alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. As used herein, “C₁₋₃ alkyl”, whether a terminal substituent or an alkylene group linking two substituents, is understood to specifically include both branched and straight-chain methyl, ethyl, and propyl.

As used herein, “alkenyl” or “alkenylene” is intended to include hydrocarbon chains of either a straight or branched configuration with one or more unsaturated carbon-carbon bonds that may occur at any stable point along the chain. Examples of “C₃₋₆alkenyl” include, but are not limited to, 1-propenyl, 2-propenyl, 1-butenyl, 2-butenyl, 3-butenyl, 3-methyl-2-butenyl, 2-pentenyl, 3-pentenyl, hexenyl, and the like.

As used herein, “alkynyl” or “alkynylene” is intended to include hydrocarbon chains of either a straight or branched configuration with one or more carbon-carbon triple bonds that may occur at any stable point along the chain, such as ethynyl, 1-propynyl, 2-propynyl, 1-butynyl, 2-butynyl, 3-butynyl, and the like.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy. Similarly, “alkylthio” or “thioalkoxy” represent an alkyl group as defined above with the indicated number of carbon atoms attached through a sulphur bridge.

As used herein, the term “aryl” is intended to mean aromatic radicals including both monocyclic aromatic radicals comprising 6 carbon atoms and polycyclic aromatic radicals comprising up to about 14 carbon atoms.

As used herein, “carbocycle” is intended to mean any stable 3- to 7-membered monocyclic or bicyclic or 7- to 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, bicyclooctane, bicyclononane, bicyclodecane (decalin), bicyclooctane, fluorenyl, phenyl, naphthyl, indanyl, adamantyl, or tetrahydronaphthyl (tetralin).

As used herein “cycloalkyl” is intended to include saturated ring groups, having the specified number of carbon atoms. For example, “C₃₋₆ cycloalkyl” denotes such groups as cyclopropyl, cyclobutyl, cyclopentyl, or cyclohexyl.

As used herein “cycloalkenyl refers to ring-containing radicals having at least one carbon-carbon double bond in the ring, and having in the range about 3 up to 12 carbons atoms.

As used herein “cycloalkynyl” refers to ring-containing radicals having at least one carbon-carbon triple bond in the ring, and having in the range about 3 up to 12 carbons atoms.

As used herein, “halo” or “halogen” refers to fluoro, chloro, bromo, and iodo. “Counterion” is used to represent a small, negatively charged species such as chloride, bromide, hydroxide, acetate, sulfate, and the like. “Haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, pentachloroethyl, 2,2,2-trifluoroethyl, 2,2-difluoroethyl, heptafluoropropyl, and heptachloropropyl. “Haloalkoxy” is intended to mean a haloalkyl group as defined above with the indicated number of carbon atoms attached through an oxygen bridge; for example trifluoromethoxy, pentafluoroethoxy, 2,2,2-trifluoroethoxy, and the like. “Halothioalkoxy” is intended to mean a haloalkyl group as defined above with the indicated number of carbon atoms attached through a sulphur bridge.

As used herein, the term “heterocyclyl” “heterocycle” “heterocycle” or “heterocyclic” refers to a ring-containing monovalent and divalent radicals having 1, 2, 3 or 4 heteroatoms, independently selected from N, O and S, as part of the ring structure and comprising at least 3 and up to about 20 atoms in the rings. Heterocyclyl groups may be aromatic, saturated or unsaturated, containing one or more double bonds. Heterocyclyl groups may contain more that one ring. The heterocyclyl rings described herein may be substituted on carbon or on a nitrogen atom if the resulting compound is stable. If specifically noted, nitrogen in the heterocycle may optionally be quaternized. It is understood that when the total number of S and O atoms in the heterocycle exceeds 1, then these heteroatoms are not adjacent to one another.

Examples of heterocyclyls include, but are not limited to, 1H-indazole, 2-pyrrolidonyl, 2H, 6H-1, 5,2-dithiazinyl, 2H-pyrrolyl, 3H-indolyl, 4-piperidonyl, 4aH-carbazole, 4H-quinolizinyl, 6H-1, 2,5-thiadiazinyl, acridinyl, azetidine, aziridine, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidine, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, thiirane, triazinyl, 1,2,3-triazolyl, 1,2,4-triazolyl, 1,2,5-triazolyl, 1,3,4-triazolyl, xanthenyl.

As used herein “host” is a living organism including but not limited to a human, animal, or mammal.

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making an acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

“Prodrugs” are intended to include any covalently bonded carriers that release the active parent drug in vivo when such prodrug is administered to a mammalian subject. Prodrugs of a compound of the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug or compound of the present invention is administered to a mammalian subject, cleaves to form a free hydroxyl, free amino, or free sulfhydryl group, respectively. Examples of prodrugs include, but are not limited to, acetate, formate and benzoate derivatives of alcohol and amine functional groups in the compounds of the present invention, and the like.

“Stable compound” and “stable structure” are meant to indicate a compound that is sufficiently robust to survive isolation to a useful degree of purity from a reaction mixture, and formulation into an efficacious therapeutic agent.

The anti-infective treatment defined herein may be applied as a sole therapy or may involve, in addition to the compound of the invention, conventional chemotherapy.

Such conjoint or concurrent treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention. Ultimately, the quantity of compound administered will be commensurate with the nature of the disease or physiological condition being treated and will be at the discretion of the physician.

Compounds of formula (I) have been shown to inhibit H. pylori MurI activity in vitro. Inhibitors of H. pylori MurI have been shown to be useful in blocking peptidoglycan biosynthesis and therefore have beneficial effects in treatment of H. pylori MurI infection. Therefore it is believed that the compounds of formula (I) may be used for the treatment of H. pylori MurI infection and diseases associated with H. pylori MurI infection. Hence compounds of formula (I) and their salts are expected to be active against H. pylori MurI infection. It is expected that the compounds of formula (I) would most likely be used as a single agent but could also be used in combination with a broad range of antimicrobial agents.

Generally, the compounds of formula (I) have been identified in at least one of the assays described below as having an IC₅₀ value of ______ micromolar or less. The preferred compounds of formula (I) have been identified in at least one of the assays described below as having an IC₅₀ value of ______ micromolar or less.

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Such methods include, but are not limited to, those described below. All references cited herein are hereby incorporated in their entirety by reference.

The novel compounds of this invention may be prepared using the reactions and techniques described herein. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are compatible with the reaction conditions, will be readily apparent to one skilled in the art and alternate methods must then be used.

Examples of such processes are illustrated below:

### EXAMPLES

Chemical abbreviations used in the Examples are defined as follows: Boc denotes t-butoxycarbonyl, Cbz denotes benzyloxycarbonyl, DCM denotes methylene chloride, DIPEA denotes diisopropylethylamine, DMF denotes N,N-dimethylformamide, DMSO denotes dimethyl sulfoxide, Et₂O denotes diethyl ether, EtOAc denotes ethyl acetate, TFA denotes trifluoroacetic acid, THF denotes tetrahydrofuran. Solvent mixture compositions are given as volume percentages or volume ratios. In cases where the NMR spectrum is complex, only diagnostic signals are reported.

Other terms used in the Examples are defined as follows: atm. denotes atmospheric pressure, equiv. denotes equivalent(s), h denotes hour(s), Tb denotes bath temperature, HPLC denotes high performance liquid chromatography, min denotes minutes, NMR denotes nuclear magnetic resonance, psi denotes pounds per square inch.

(i) temperatures are given in degrees Celsius (° C.); unless otherwise stated, operations were carried out at room or ambient temperature, that is, at a temperature in the range of 18-25° C.;

(ii) organic solutions were dried over anhydrous magnesium sulfate or sodium sulfate; evaporation of solvent was carried out using a rotary evaporator under reduced pressure (600-4000 Pascals; 4.5-30 mm Hg) with a bath temperature of up to 60° C.;

(iii) chromatography means flash chromatography on silica gel or by FlashMaster™ II by Jones Chromatography using Isolute columns; thin layer chromatography (TLC) was carried out on silica gel plates;

(iv) in general, the course of reactions was followed by TLC or analytical HPLC and reaction times are given for illustration only;

(v) melting points are uncorrected and (dec) indicates decomposition;

(vi) final products had satisfactory proton nuclear magnetic resonance (NMR) spectra;

(vii) when given, NMR data is in the form of delta values for major diagnostic protons, given in parts per million (ppm) relative to tetramethylsilane (TMS) as an internal standard, determined at 300 or 500 MHz using deuterated chloroform (CDCl₃) or DMSO-_{d6} or CD₃OD as a solvent; conventional abbreviations for signal shape are used; for AB spectra the directly observed shifts are reported; coupling constants (J) are given in Hz; Ar designates an aromatic proton when such an assignment is made;

(viii) reduced pressures are given as absolute pressures in pascals (Pa); elevated pressures are given as gauge pressures in bars;

(ix) solvent ratios are given in volume:volume (v/v) terms; and

(x) Mass spectra (MS) were run using an automated system with atmospheric pressure electrospray ionization (ESI). Generally, only spectra where parent masses are observed are reported. The lowest mass major ion is reported for molecules where isotope splitting results in multiple mass spectral peaks (for example when chlorine is present).

The invention will now be illustrated by the following non-limiting examples.

### Example 1

### 5-(4-fluorophenyl)-N-methyl-3-phenyl-3H-1,4-benzodiazepin-2-amine

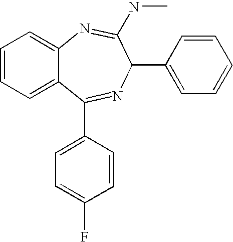

Step 1. Preparation of 2-[(4-fluorophenyl)(imino)methyl]aniline: A solution of 4-bromofluorobenzene (10 mL, 91 mmol) in 20 mL THF was added slowly to 2.0 g (87 mmol) of magnesium turnings and a grain of I₂ in 80 mL THF. After 2 hours of stirring at room temperature, 3.4 g (29 mmol) of anthranilonitrile were added, and the mixture was heated to 40° C. for 3 hours. The mixture was quenched with NH₄Cl (aqueous) and partitioned between water and EtOAc. The EtOAc was separated and washed with brine. Drying (MgSO₄) and removal of solvent gave a yellow solid, ¹H NMR (300 MHz, DMSO-D6) δ ppm 6.3-7.6 (m, 8H) 10.3 (s, 1H).

Step 2. Preparation of 5-(4-fluorophenyl)-3-phenyl-1,3-dihydro-2H-1,4-benzodiazepin-2-one: A solution of 2-[(4-fluorophenyl)(imino)methyl]aniline (560 mg, 2.6 mmol) and 2-methoxy-2-oxo-1-phenylethanaminium chloride (527 mg, 2.6 mmol) in 3 mL ethanol was heated at 80° C. for 20 minutes in a microwave reactor. Methanesulfonic acid (150 μL) was added via syringe and the solution was heated at 80° C. for 20 minutes in a microwave reactor. The mixture was diluted with EtOAc and washed with Na₂CO₃ (aqueous), water and brine. Drying (MgSO₄) and removal of solvent gave an oily solid that was triturated with ether to give 110 mg of product as a white solid. The mother liquor was concentrated and chromatographed on silica gel (CH₂Cl₂ followed by gradient elution to 5% MeOH in CH₂Cl₂) to give an additional 140 mg of product, ¹H NMR (300 MHz, DMSO-D6) δ ppm 4.76 (s, 1H) 7.25-7.40 (m, 8H) 7.54-7.68 (m, 5H) 10.78 (s, 1H).

Step 3. Preparation of 5-(4-fluorophenyl)-N-methyl-3-phenyl-3H-1,4-benzodiazepin-2-amine:

A solution (310 μL) of 1M TiCl₄ in CH₂Cl₂ was added to a solution of 5-(4-fluorophenyl)-3-phenyl-1,3-dihydro-2H-1,4-benzodiazepin-2-one (101 mg, 0.31 mmol) in 5 mL of 2N MeNH₂ in THF. The mixture was heated at 80° C. for 20 minutes in a microwave reactor. The mixture was diluted with EtOAc and washed with water and brine. Drying (MgSO₄) and removal of solvent was followed by chromatography on silica gel (CH₂Cl₂ followed by gradient elution to 30% EtOAc in CH₂Cl₂) to give 80 mg of product, ¹H NMR (300 MHz, DMSO-D6) δ ppm 2.65 (d, 3H) 5.62-5.71 (m, 1H) 7.00 (t, 1H) 7.20-7.34 (m, 4H) 7.37-7.54 (m, 4H) 7.62 (dd, 2H) 7.72 (d, 2H).

### Example 2

### 5-(4-fluorophenyl)-N-methyl-3-thien-2-yl-3H-1,4-benzodiazepin-2-amine

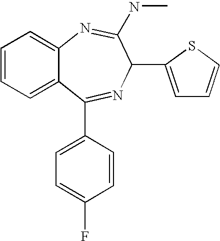

Step 1. Preparation of 5-(4-fluorophenyl)-3-thien-2-yl-1,3-dihydro-2H-1,4-benzodiazepin-2-one Following the procedures of Step 2 of Example 1, 2-[(4-fluorophenyl)(imino)methyl]aniline (548 mg, 2.6 mmol) and 2-methoxy-2-oxo-1-thien-2-ylethanaminium chloride (531 mg, 2.5 mmol) were converted to 290 mg of product, ¹H NMR (300 MHz, DMSO-D6) δ ppm 5.08 (s, 1H) 7.05 (dd, J=4.90, 3.58 Hz, 1H) 7.10 (d, J=3.39 Hz, 1H) 7.15-7.44 (m, 5H) 7.46-7.72 (m, 4H) 10.81 (s, 1H).

Step 2. Preparation of 5-(4-fluorophenyl)-N-methyl-3-thien-2-yl-3H-1,4-benzodiazepin-2-amine: Following the procedure of Step 3 of Example 1, 5-(4-fluorophenyl)-3-thien-2-yl-1,3-dihydro-2H-1,4-benzodiazepin-2-one (104 mg, 0.31 mmol) was converted to the title compound, ¹H NMR (300 MHz, DMSO-D6) δ ppm 2.69 (d, J=4.71 Hz, 3H) 4.70 (s, 1H) 6.09 (q, J=4.71 Hz, 1H) 6.94-7.06 (m, 1H) 7.14-7.28 (m, 4H) 7.30-7.34 (m, 2H) 7.45-7.55 (m, 1H) 7.56-7.65 (m, 3H).

### Example 3

### 5-(4-fluorophenyl)-3-phenyl-3H-1,4-benzodiazepin-2-amine

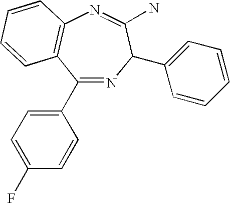

A solution of 2-[(4-fluorophenyl)(imino)methyl]aniline (582 mg, 2.7 mmol) and cyano(phenyl)methanaminium chloride (455 mg, 2.7 mmol) in 2 mL ethanol was heated was heated at 80° C. for 20 minutes in a microwave reactor. Methanesulfonic acid (300 μL) was added via syringe and the solution was stirred at room temperature overnight. The mixture was diluted with EtOAc and washed with Na₂CO₃ (aqueous), water and brine. Drying (MgSO₄) and removal of solvent gave an oil. Purification by reverse phase HPLC (35-75% gradient of CH₃CN in water with 0.1% TFA) gave impure product. Further purification by chromatography on silica gel (CH₂Cl₂ followed by gradient elution to 5% MeOH in CH₂Cl₂) gave 105 mg of product as a white solid, ¹H NMR (300 MHz, DMSO-D6) δ ppm 4.28-4.43 (m, 1H) 4.58-5.56 (m, 2H) 7.00 (t, 1H) 7.15-7.36 (m, 4H) 7.36-7.44 (m, 1H) 7.45-7.56 (m, 3H) 7.56-7.67 (m, 4H) 7.67-7.79 (m, 2H).

### Example 4

### 2-azetidin-1-yl-5-(4-fluorophenyl)-3-phenyl-3H-1,4-benzodiazepine

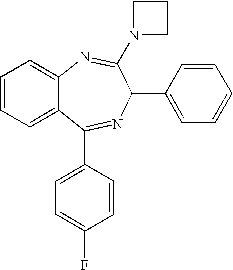

Following the procedures of Step 3 of Example 1, 5-(4-fluorophenyl)-3-phenyl-1,3-dihydro-2H-1,4-benzodiazepin-2-one (85 mg, 0.24 mmol) and azetidine (200 μL) in 5 mL THF were converted to the title compound, ¹H NMR (300 MHz, DMSO-D6) δ ppm 1.81-2.04 (m, 2H) 3.35-4.19 (m, 4H) 4.26-4.40 (m, 1H) 6.97-7.06 (m, 1H) 7.19-7.40 (m, 5H) 7.40-7.55 (m, 3H) 7.63-7.72 (m, 2H) 7.74-7.90 (m, 2H).

### Example 5

### N-ethyl-5-(4-fluorophenyl)-3-phenyl-3H-1,4-benzodiazepin-2-amine

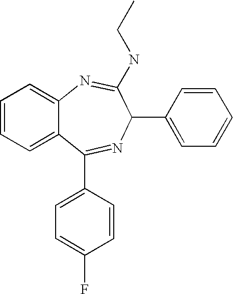

Following the procedures of Step 3 of Example 1, 5-(4-fluorophenyl)-3-phenyl-1,3-dihydro-2H-1,4-benzodiazepin-2-one (85 mg, 0.24 mmol) and 2 N ethylamine in 5 mL THF were converted to the title compound, ¹H NMR (300 MHz, DMSO-D6) δ ppm 0.86-1.01 (m, 3H) 3.05-3.28 (m, 2H) 4.34-4.51 (m, 1H) 5.38-5.59 (m, 1H) 6.93-7.06 (m, 1H) 7.12-7.38 (m, 5H) 7.35-7.57 (m, 4H) 7.56-7.65 (m, 2H) 7.66-7.78 (m, 2H).

### Example 6

### N-methyl-5-(4-methylphenyl)-3-thien-2-yl-3H-pyrido[2,3 e][1,4]diazepin-2-amine

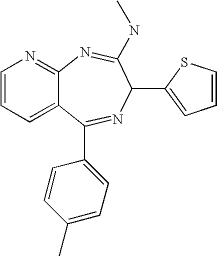

Step 1. Preparation of 3-[imino(4-methylphenyl)methyl]pyridin-2-amine: A mixture of (4-methylphenyl)magnesium bromide (25 mL of a 1 M solution) and 2-amino-3-cyanopyridine (1.0 g, 8.4 mmol) in THF was stirred at room temperature overnight. The mixture was quenched with NH₄Cl (aqueous) and partitioned between water and EtOAc. The EtOAc was separated and washed with brine. Drying (MgSO₄) and removal of solvent gave a brown solid, ¹H NMR (300 MHz, DMSO-D6) δ ppm 2.36 (s, 3H) 6.49 (m, 1H) 7.18-7.30 (m, 5H) 7.93-8.08 (m, 2H) 10.45 (s, 1H).

Step 2. Preparation of 5-(4-methylphenyl)-3-thien-2-yl-1,3-dihydro-2H-pyrido[2,3-e][1,4]diazepin-2-one: A solution of 3-[imino(4-methylphenyl)methyl]pyridin-2-amine (400 mg, 1.9 mmol) and 2-methoxy-2-oxo-1-thien-2-ylethanaminium chloride (393 mg, 1.9 mmol) (ref. DE 2204117) in 5 mL of MeOH was heated at 80° C. for 20 minutes in a microwave reactor. DBU (850 μL) was added via syringe and the solution was heated at 80° C. for 20 minutes in a microwave reactor. The mixture was diluted with EtOAc and washed with Na₂CO₃ (aqueous), water and brine. Drying (MgSO₄) and removal of solvent gave an oily solid that was triturated with methanol to give 300 mg of product as a white solid. The mother liquor was concentrated and chromatographed on silica gel (CH₂Cl₂ followed by gradient elution to 5% MeOH in CH₂Cl₂) to give an additional 110 mg of product, ¹H NMR (300 MHz, DMSO-D6) δ ppm 2.44 (s, 3H) 5.24 (s, 1H) 7.09-7.20 (m, 2H) 7.35-7.42 (m, 3H) 7.45-7.52 (m, 2H) 7.6 (m, 1H) 7.88 (dd, J=7.82, 1.60 Hz, 1H) 8.74 (dd, J=4.62, 1.60 Hz, 1H) 11.25 (s, 11H).

Step 3. Preparation of N-methyl-5-(4-methylphenyl)-3-thien-2-yl-3H-pyrido[2,3-e][1,4]diazepin-2-amine: Following the procedures of Step 3 of Example 1, 5-(4-methylphenyl)-3-thien-2-yl-1,3-dihydro-2H-pyrido[2,3-e][1,4]diazepin-2-one (70 mg, 0.21 mmol) was converted to the title compound, ¹H NMR (300 MHz, DMSO-D6) δ ppm 2.37 (s, 3H) 2.71 (d, J=4.52 Hz, 3H) 4.76 (s, 1H) 6.35 (q, J=4.71 Hz, 1H) 7.00 (dd, J=7.82, 4.62 Hz, 1H) 7.14-7.25 (m, 2H) 7.29 (d, J=8.10 Hz, 2H) 7.46 (d, J=8.10 Hz, 2H) 7.59-7.71 (m, 2H) 8.59 (dd, J=4.52, 1.88 Hz, 1H).

### Example 7

### 5-(4-methoxyphenyl)-N-methyl-3-thien-2-yl-3H-pyrido[2,3-e][1,4]diazepin-2-amine

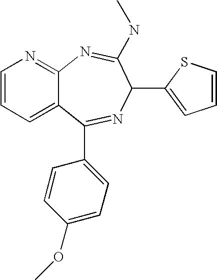

Step 1. Preparation of 3-[imino(4-methoxyphenyl)methyl]pyridin-2-amine: A mixture of (4-methoxyphenyl)magnesium bromide (25 mL of a 1 M solution) and 2-amino-3-cyanopyridine (1.0 g, 8.4 mmol) in THF was stirred at room temperature overnight. LC-MS analysis showed the presence of starting material. Additional (4-methoxyphenyl)magnesium bromide (10 mL of a 1 M solution) was added, and the mixture was stirred at room temperature overnight. Again, LC-MS analysis showed the presence of starting material, and additional (4-methoxyphenyl)magnesium bromide (10 mL of a 1 M solution) was added. After stirring at room temperature overnight, the mixture was quenched with NH₄Cl (aqueous) and partitioned between water and EtOAc. The EtOAc was separated and washed with brine. Drying (MgSO₄) and removal of solvent was followed by purification on a Biotage NH₂ column (100% hexanes followed by gradient elution to 100% CH₂Cl₂) to afford product as a brown solid, ¹H NMR (300 MHz, DMSO-D6) δ ppm 3.8 (s, 3H) 6.50 (dd, J=7.72, 4.71 Hz, 1H) 7.02 (d, J=8.67 Hz, 2H) 7.28 (m, 3H) 8.04 (dd, J=4.71, 1.88 Hz, 1H) 10.43 (s, 1H).

Step 2. Preparation of 5-(4-methoxyphenyl)-3-thien-2-yl-1,3-dihydro-2H-pyrido[2,3-e][1,4]diazepin-2-one: A solution of 3-[imino(4-methoxyphenyl)methyl]pyridin-2-amine (476 mg, 2.1 mmol) and 2-methoxy-2-oxo-1-thien-2-ylethanaminium chloride (435 mg, 2.1 mmol) in 5 mL of MeOH was heated at 80° C. for 20 minutes in a microwave reactor. DBU (630 μL) was added via syringe and the solution was heated at 80° C. for 30 minutes in a microwave reactor. Insoluble solids were filtered and rinsed with methanol to afford 320 mg of product as a white solid, ¹H NMR (300 MHz, DMSO-D6) δ ppm 3.82 (s, 3H) 5.14 (s, 1H) 6.95-7.18 (m, 4H) 7.30 (dd, J=7.72, 4.71 Hz, 1H) 7.39-7.63 (m, 3H) 7.85 (dd, J=7.72, 1.32 Hz, 1H) 8.67 (dd, J=4.62, 1.60 Hz, 1H) 11.17 (s, 1H).

Step 3. Preparation of 5-(4-methoxyphenyl)-N-methyl-3-thien-2-yl-3H-pyrido[2,3-e][1,4]diazepin-2-amine: Following the procedures of Step 3 of Example 1, 5-(4-methylphenyl)-3-thien-2-yl-1,3-dihydro-2H-pyrido[2,3-e][1,4]diazepin-2-one (84 mg, 0.24 mmol) was converted to the title compound, ¹H NMR (300 MHz, DMSO-D6) δ ppm 2.77 (d, J=4.52 Hz, 3H) 3.88 (s, 3H) 4.81 (s, 1H) 6.39 (q, J=4.71 Hz, 1H) 7.03-7.15 (m, 3H) 7.20-7.29 (m, 2H) 7.58 (d, J=8.67 Hz, 2H) 7.64-7.77 (m, 2H) 8.65 (dd, J=4.71, 1.88 Hz, 1H).

### Example 8

### N-cyclopropyl-5-(4-methylphenyl)-3-thien-2-yl-3H-pyrido[2,3-e][1,4]diazepin-2-amine

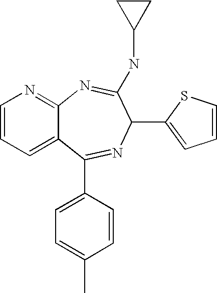

Following the procedures of Step 3 of Example 1, 5-(4-methylphenyl)-3-thien-2-yl-1,3-dihydro-2H-pyrido[2,3-e][1,4]diazepin-2-one (70 mg, 0.21 mmol) and 70 μL cyclopropylamine in 5 mL THF was converted to the title compound, ¹H NMR (300 MHz, DMSO-D6) δ ppm 0.25-0.38 (m, 1H) 0.50-0.65 (m, 3H) 2.38 (s, 3H) 2.72-2.84 (m, 1H) 4.72 (s, 1H) 6.13 (d, J=3.77 Hz, 1H) 7.02 (dd, J=7.82, 4.62 Hz, 1H) 7.13-7.26 (m, 2H) 7.30 (d, J=7.91 Hz, 2H) 7.47 (d, J=8.10 Hz, 2H) 7.57-7.71 (m, 2H) 8.61 (dd, J=4.43, 1.79 Hz, 1H).

### Example 9

### N-methyl-3-pyridin-4-yl-5-thien-2-yl-3H-pyrido[2,3-e][1,4]diazepin-2-amine

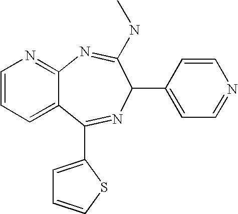

Step 1. Preparation of 3-[imino(thien-2-yl)methyl]pyridin-2-amine: 2-Amino-3-cyanopyridine (3 g, 25 mmol) was added to 75 mL (75 mmol) of a 1.0 M solution of 2-thienyllithium in THF, and the mixture was stirred at room temperature overnight. After quenching with NH₄Cl (aqueous), the mixture was partitioned between EtOAc and water. The EtOAc was separated and washed with brine. The combined aqueous layers were extracted with EtOAc, which was washed with brine. The combined EtOAc layers were dried (MgSO₄) and concentrated to give an oil. Trituration of the oil with ether gave yellow solids that were filtered, rinsed well with ether and dried in vacuo affording 3.3 g of product as a 3:2 mixture of geometrical isomers, ¹H NMR (300 MHz, DMSO-D6) δ ppm 5.69 and 7.68 (2s, 2H) 6.49-6.68 (m, 1H) 7.05-7.4 (m, 2H) 7.59-7.70 (m, 2H) 7.99-8.11 (m, 1H) 10.27 and 10.69 (2s, 1H, 2:3 ratio).

Step 2. Preparation of 3-pyridin-4-yl-5-thien-2-yl-1,3-dihydro-2H-pyrido[2,3-e][1,4]diazepin-2-one: A solution of 3-[imino(thien-2-yl)methyl]pyridin-2-amine (326 mg, 1.5 mmol) and 2-ethoxy-2-oxo-1-pyridin-4-ylethanaminium chloride (300 mg, 1.5 mmol) (ref. U.S. Pat. No. 6,191,166 B1) in 2 mL methanol was heated at 80° C. for 20 minutes in a microwave reactor. DBU (1 mL) was added via syringe and the solution was heated at 80° C. for 30 minutes in a microwave reactor. The mixture was diluted with EtOAc and washed with water and brine. Drying (MgSO₄) and removal of solvent was followed by trituration with methanol to give 330 mg of a white solid, ¹H NMR (300 MHz, DMSO-D6) δ ppm 5.02 (s, H) 7.17 (d, J=3.01 Hz, 2H) 7.38 (dd, J=7.72, 4.71 Hz, 1H) 7.61 (d, J=5.46 Hz, 2H) 7.77-7.89 (m, 1H) 8.21 (d, 1H) 8.61 (d, J=5.65 Hz, 2H) 8.70 (d, 1H) 11.27 (s, 1H).

Step 3. Preparation of N-methyl-3-pyridin-4-yl-5-thien-2-yl-3H-pyrido[2,3-e][1,4]diazepin-2-amine: Following the procedures of Step 3 of Example 1, 3-pyridin-4-yl-5-thien-2-yl-1,3-dihydro-2H-pyrido[2,3-e][1,4]diazepin-2-one (80 mg, 0.25 mmol) was converted to the title compound, ¹H NMR (300 MHz, DMSO-D6) δ ppm 2.66 (d, J=4.52 Hz, 3H) 4.63 (s, 1H) 6.25 (q, J=4.52 Hz, 1H) 7.08 (dd, J=7.82, 4.62 Hz, 1H) 7.12-7.22 (m, 2H) 7.71 (d, J=5.65 Hz, 2H) 7.78 (dd, J=4.90, 1.13 Hz, 1H) 8.05 (dd, J=7.82, 1.98 Hz, 1H) 8.63 (dd, J=4.52, 1.88 Hz, 1H) 8.68 (d, J=5.84 Hz, 2H)

### Example 10

### N-ethyl-3-pyridin-4-yl-5-thien-2-yl-3H-pyrido[2,3-e][1,4]diazepin-2-amine

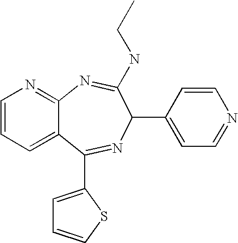

Following the procedures of Step 3 of Example 1, 3-pyridin-4-yl-5-thien-2-yl-1,3-dihydro-2H-pyrido[2,3-e][1,4]diazepin-2-one (80 mg, 0.25 mmol) and 5 mL of 2 N ethylamine in THF was converted to the title compound, ¹H NMR (300 MHz, DMSO-D6) δ ppm 0.97 (t, J=7.06 Hz, 3H) 3.1-3.25 (m, 2H) 4.60 (s, 1H) 6.23 (t, J=5.84 Hz, 1H) 7.07 (dd, J=7.82, 4.62 Hz, 1H) 7.12-7.22 (m, 2H) 7.70 (d, J=5.84 Hz, 2H) 7.78 (dd, J=4.99, 1.04 Hz, 1H) 8.05 (dd, J=7.82, 1.98 Hz, 1H) 8.62 (dd, J=4.62, 1.98 Hz, 1H) 8.68 (d, J=5.84 Hz, 2H).

### Example 11

### N-cyclopropyl-3-pyridin-4-yl-5-thien-2-yl-3H-pyrido[2,3-e][1,4]diazepin-2-amine

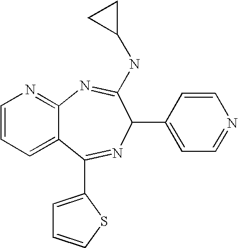

Following the procedures of Step 3 of Example 1, 3-pyridin-4-yl-5-thien-2-yl-1,3-dihydro-2H-pyrido[2,3-e][1,4]diazepin-2-one (80 mg, 0.25 mmol) and 140 μL cyclopropylamine in 5 mL THF was converted to the title compound, ¹H NMR (300 MHz, DMSO-D6) δ ppm 0.25-0.39 (m, 1H) 0.48-0.64 (m, 3H) 2.79 (m, 1H) 4.57 (s, 1H) 6.26 (d, J=3.96 Hz, 1H) 7.10 (dd, J=7.82, 4.62 Hz, 1H) 7.13-7.23 (m, 2H) 7.67 (d, J=5.46 Hz, 2H) 7.78 (dd, J=4.90, 1.13 Hz, 1H) 8.06 (dd, J=7.82, 1.98 Hz, 1H) 8.61-8.69 (m, 3H).

### Example 12

### 3-(2-amino-1,3-thiazol-4-yl)-N-methyl-5-thien-2-yl-3H-pyrido[2,3-e][1,4]diazepin-2-amine

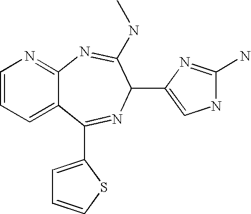

Step 1. Preparation of 3-(2-amino-1,3-thiazol-4-yl)-5-thien-2-yl-1,3-dihydro-2H-pyrido[2,3-e][1,4]diazepin-2-one: Following the procedure of Step 2 of Example 9, 3-[imino(thien-2-yl)methyl]pyridin-2-amine (191 mg, 0.94 mmol) and 1-(2-amino-1,3-thiazol-4-yl)-2-ethoxy-2-oxoethanaminium chloride (224 mg, 0.94 mmol) (ref. U.S. 78-870932) was converted to product, ¹H NMR (300 MHz, DMSO-D6) δ ppm 4.58 (s, 1H) 6.76 (s, 1H) 6.87 (s, 2H) 7.10-7.19 (m, 2H) 7.34 (dd, J=7.72, 4.71 Hz, 1H) 7.81 (dd, J=4.43, 1.60 Hz, 1H) 8.17 (d, J=7.72 Hz, 1H) 8.67 (d, J=3.20 Hz, 1H) 11.12 (s, 1H).

Step 2. Preparation of 3-(2-amino-1,3-thiazol-4-yl)-N-methyl-5-thien-2-yl-3H-pyrido[2,3-e][1,4]diazepin-2-amine: Following the procedure of Step 3 of Example 1, 3-(2-amino-1,3-thiazol-4-yl)-5-thien-2-yl-1,3-dihydro-2H-pyrido[2,3-e][1,4]diazepin-2-one (88 mg, 0.26 mmol) was converted to the title compound, ¹H NMR (300 MHz, DMSO-D6) δ ppm 2.72 (d, J=4.52 Hz, 3H) 4.26 (s, 1H) 6.75-6.89 (m, 2H) 6.95-7.10 (m, 2H) 7.10-7.19 (m, 3H) 7.74 (dd, J=4.05, 2.17 Hz, 1H) 8.01 (dd, J=7.72, 1.88 Hz, 1H) 8.58 (dd, J=4.62, 1.98 Hz, 1H).

### Example 13

### 5-(6-aminopyridin-3-yl)-N-methyl-3-thien-2-yl-3H-pyrido[2,3-e][1,4]diazepin-2-amine

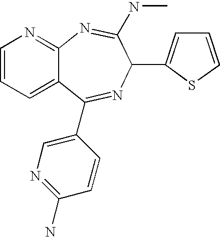

Step 1. Preparation of (2-fluoropyridin-3-yl)(6-fluoropyridin-3-yl)methanol: A solution of 1.6 N n-butyllithium in hexanes (21 mL, 34 mmol) was added slowly to a solution of 5-bromo-2-fluoropyridine (5.0 g, 28 mmol) in 150 mL ether cooled in a dry ice-acetone bath. After stirring 45 minutes, 2-fluoro-3-pyridine carboxaldehyde was added via syringe, and the mixture was stirred 30 min. The solution was allowed to warm to room temperature as it was quenched with NH₄Cl (aqueous). The solution was diluted with EtOAc, washed with water and brine, dried (MgSO₄) and concentrated yielding 6.2 g of an oil, ¹H NMR (300 MHz, DMSO-D6) δ ppm 5.99 (d, J=4.14 Hz, 1H) 6.46 (d, J=4.33 Hz, 1H) 7.16 (dd, J=8.48, 2.83 Hz, 1H) 7.37-7.50 (m, 1H) 7.81-8.00 (m, 1H) 8.06-8.23 (m, 2H) 8.26 (d, J=1.32 Hz, 1 H).

Step 2. Preparation of (2-fluoropyridin-3-yl)(6-fluoropyridin-3-yl)methanone: A mixture of (2-fluoropyridin-3-yl)(6-fluoropyridin-3-yl)methanol in 100 mL THF and 3.4 g of MnO₂ was heated at reflux overnight. Additional (1.0 g) MnO₂ was added, and heating was continued for 10 hours. Solids were removed by filtering through Celite and rinsing well with EtOAc. The filtrate was concentrated to give 6.2 g of product as an oil, ¹H NMR (300 MHz, DMSO-D6) δ ppm 7.42 (dd, J=8.67, 2.64 Hz, 1H) 7.60 (ddd, J=7.30, 5.04, 1.98 Hz, 1H) 8.28 (ddd, J=9.56, 7.49, 1.98 Hz, 1H) 8.43 (td, J=8.19, 2.45 Hz, 1H) 8.54 (d, J=4.90 Hz, 1H) 8.69 (d, J=2.45 Hz, 1H).

Step 3. Preparation of 3-[imino(6-amino-pyridin-3-yl)methyl]pyridin-2-amine: A solution of (2-fluoropyridin-3-yl)(6-fluoropyridin-3-yl)methanone (3.9 g, 18 mmol) in 70 mL of 7N NH₃ in methanol was heated at 120° C. in a pressure vessel overnight. Ti(IV) isopropoxide (10.6 mL, 36 mmol) was added, and the mixture was heated at 100° C. for 6 hours. The mixture was partitioned between water and EtOAc. The EtOAc was separated and washed with brine. The combined aqueous layers were extracted 5 times with EtOAc, each extract being washed with brine. The combined EtOAc extracts were dried (MgSO₄) and concentrated to give an oil that was purified on a Biotage NH₂ column (100% hexanes followed by gradient elution to 100% CH₂Cl₂ and then to 10% MeOH in CH₂Cl₂) to give 1.4 g of product as a yellow solid, ¹H NMR (300 MHz, DMSO-D6) δ ppm 6.4 (s, 1H) 6.5 (m, 2H) 7.4 (m, 1H) 7.7 (m, 1H) 7.8 (s, 1H) 7.9 (s, 1H) 8.0 (m, 1H) 8.2 (m, 1H) 10.4 (s, 1H).

Step 4. Preparation of 5-(6-aminopyridin-3-yl)-3-thien-2-yl-1,3-dihydro-2H-pyrido[2,3-e][1,4]diazepin-2-one: Following the procedure of Step 2 of Example 7, 3-[imino(6-amino-pyridin-3-yl)methyl]pyridin-2-amine (678 mg, 2.1 mmol) was converted to product, ¹H NMR (300 MHz, DMSO-D6) δ ppm 5.07 (s, 1H) 6.59 (s, 2H) 7.00-7.14 (m, 2H) 7.31 (dd, J=7.72, 4.71 Hz, 1H) 7.52 (d, J=4.90 Hz, 1H) 7.63 (dd, J=8.76, 2.35 Hz, 1H) 7.85-8.05 (m, 2H) 8.66 (dd, J=4.52, 1.51 Hz, 1H) 11.12 (s, 1H).

Step 5. Preparation of 5-(6-aminopyridin-3-yl)-N-methyl-3-thien-2-yl-3H-pyrido[2,3-e][1,4]diazepin-2-amine: Following the procedure of Step 3 of Example 1, 5-(6-aminopyridin-3-yl)-3-thien-2-yl-1,3-dihydro-2H-pyrido[2,3-e][1,4]diazepin-2-one (110 mg, 2.1 mmol) was converted to the title product, ¹H NMR (300 MHz, DMSO-D6) δ ppm 2.71 (d, J=4.52 Hz, 3H) 4.70 (s, 1H) 6.30 (q, J=4.71 Hz, 1H) 6.43-6.57 (m, 3H) 7.01 (dd, J=7.72, 4.71 Hz, 1H) 7.11-7.22 (m, 2H) 7.56-7.65 (m, 2H) 7.75 (dd, J=7.82, 1.79 Hz, 1H) 8.04 (d, J=2.26 Hz, 1H) 8.58 (dd, J=4.52, 1.88 Hz, 1H).

### Example 14

### 5-(2-aminopyridin-4-yl)-N-methyl-3-thien-2-yl-3H-pyrido[2,3-e][1,4]diazepin-2-amine

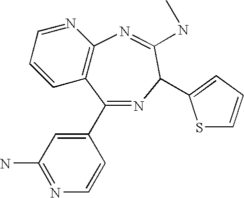

Step 1. Preparation of (2-fluoropyridin-3-yl)(2-fluoropyridin-4-yl)methanol: Following the procedure of Step 3 of Example 7, 4-bromo-2-fluoropyridine (2.16 g, 12 mmol) was reacted with 2-fluoro-3-pyridine carboxaldehyde. Chromatography on silica gel (100% CH₂Cl₂ followed by gradient elution to 20% EtOAc in CH₂Cl₂) afforded 1.6 g of product as a white solid, ¹H NMR (300 MHz, DMSO-D6) δ ppm 5.96 (d, J=4.33 Hz, 1H) 6.59 (d, J=4.33 Hz, 1H) 7.17 (s, 1H) 7.29 (d, J=5.09 Hz, 1H) 7.35-7.44 (m, 1H) 8.00-8.10 (m, 1H) 8.19 (dd, J=5.18 Hz, 2H).

Step 2. Preparation of (2-fluoropyridin-3-yl)(2-fluoropyridin-4-yl)methanone: A mixture of (2-fluoropyridin-3-yl)(2-fluoropyridin-4-yl)methanol (1.6 g, 7.3 mmol) and MnO₂ (3.8 g) in 50 mL THF was heated at 70° C. overnight. The mixture was filtered through Celite, which was rinsed well with EtOAc. Removal of solvent from the filtrate gave 1.5 g of product as a white solid, ¹H NMR (300 MHz, CHLOROFORM-D) δ ppm 7.19 (d, J=4.52 Hz, 1H) 7.34-7.41 (m, 1H) 7.43 (d, J=3.77 Hz, 1H) 8.09 (t, J=8.29 Hz, 1H) 8.37 (d, J=5.28 Hz, 1H) 8.44 (d, J=5.28 Hz, 1H).

Step 3. Preparation of 3-[imino(2-aminopyridin-4-yl)methyl]pyridin-2-amine: A solution of (2-fluoropyridin-3-yl)(2-fluoropyridin-4-yl)methanone (1.5 g, 6.8 mmol) in 40 mL 7 N NH₃ in MeOH was heated in a pressure vessel at 110° C. overnight. As reaction was incomplete as monitored by LC-MS, heating was continued at 140° C. for 8 hours. Ti(IV) isopropoxide (4 mL, 13.6 mmol) was added, and the mixture was heated at 100° C. for 6 hours. The mixture was partitioned between water and EtOAc and filtered through Celite, rinsing through with water and EtOAc. The EtOAc was separated and washed with brine. The combined aqueous layers were extracted 3 more times with EtOAc, each extract being washed with brine. The combined EtOAc extracts were dried (MgSO₄) and solvent was removed to give a yellow solid. Trituration of the solid with ether gave 550 mg of product, ¹H NMR (300 MHz, DMSO-D6) δ ppm 6.14 (s, 1H) 6.29 (s, 1H) 6.38 (d, J=5.09 Hz, 1H) 6.52 (dd, J=7.72, 4.71 Hz, 1H) 7.28 (d, J=7.72 Hz, 1H) 7.97 (d, J=5.09 Hz, 1H) 8.00-8.26 (m, 2H) 10.56 (s, 1H).

Step 4. Preparation of 5-(2-aminopyridin-4-yl)-3-thien-2-yl-1,3-dihydro-2H-pyrido[2,3-e][1,4]diazepin-2-one: Following the procedure of Step 2 of Example 6, 3-[imino(2-aminopyridin-4-yl)methyl]pyridin-2-amine (550 mg, 2.6 mmol) was converted to product. Purification was carried out via reverse phase HPLC (10-35% gradient of CH₃CN in water with 0.1% TFA) to afford clean product after free basing from the conjugate TFA salt with NaHCO₃ and extraction into EtOAc, ¹H NMR (300 MHz, DMSO-D6) δ ppm 5.23 (s, 1H) 6.18 (s, 2H) 6.46-6.62 (m, 2H) 7.05 (dd, J=4.99, 3.49 Hz, 1H) 7.12 (d, J=3.39 Hz, 1H) 7.33 (dd, J=7.91, 4.71 Hz, 1H) 7.55 (dd, J=5.09, 1.32 Hz, 1H) 7.86 (dd, J=7.91, 1.70 Hz, 1H) 7.99 (d, J=5.09 Hz, 1H) 8.69 (dd, J=4.71, 1.70 Hz, 1H) 11.27 (s, 1H).

Step 5. Preparation of 5-(2-aminopyridin-4-yl)-N-methyl-3-thien-2-yl-3H-pyrido[2,3-e][1,4]diazepin-2-amine: Following the procedure of Step 3 of Example 1, 5-(2-aminopyridin-4-yl)-3-thien-2-yl-1,3-dihydro-2H-pyrido[2,3-e][1,4]diazepin-2-one (100 mg, 2.0 mmol) was converted to the title product, ¹H NMR (300 MHz, DMSO-D6) δ ppm 2.71 (d, J=4.71 Hz, 3H) 4.79 (s, 1H) 6.13 (s, 2H) 6.39 (q, J=4.52 Hz, 1H) 6.52 (s, 1H) 6.62 (dd, J=5.27, 1.32 Hz, 1H) 7.02 (dd, J=7.82, 4.62 Hz, 1H) 7.17 (dd, J=4.99, 3.48 Hz, 1H) 7.23 (d, J=3.39 Hz, 1H) 7.63 (dd, J=4.99, 1.04 Hz, 1H) 7.67 (dd, J=7.72, 1.88 Hz, 1H) 7.99 (d, J=5.27 Hz, 1H) 8.60 (dd, J=4.52, 1.88 Hz, 1H).

### Example 15

### 5-(2-aminopyridin-4-yl)-N-cyclopropyl-3-thien-2-yl-3H-pyrido[2,3-e][1,4]diazepin-2-amine

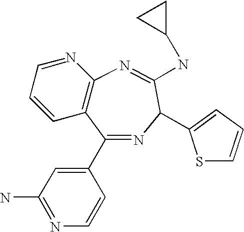

Following the procedures of Step 3 of Example 1, 5-(2-aminopyridin-4-yl)-N-methyl-3-thien-2-yl-3H-pyrido[2,3-e][1,4]diazepin-2-amine (100 mg, 0.20 mmol) and 700 μL cyclopropylamine in 5 mL THF was converted to the title compound, ¹H NMR (300 MHz, DMSO-D6) δ ppm 0.26-0.38 (m, 1H) 0.50-0.66 (m, 3H) 2.76-2.83 (m, 1H) 4.74 (s, 1H) 6.07-6.21 (m, 3H) 6.46-6.57 (m, 1H) 6.59-6.65 (m, 1H) 7.05 (dd, J=7.82, 4.62 Hz, 1H) 7.11-7.20 (m, 1H) 7.22 (d, J=3.39 Hz, 1H) 7.61 (d, J=4.90 Hz, 1H) 7.65-7.73 (m, 1H) 8.00 (d, J=5.46 Hz, 1H) 8.61 (dd, J=4.52, 1.88 Hz, 1H).

### Example 16

### 3-(3-chlorothien-2-yl)-5-(2-furyl)-N-methyl-3H-pyrido[2,3-e][1,4]diazepin-2-amine

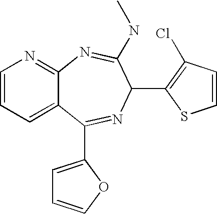

Step 1. Preparation of ethyl (3-chlorothien-2-yl)(oxo)acetate: A solution of 3-chlorothiophene (5 μL, 54 mmol) in 60 mL THF was cooled in a dry ice-acetone bath before slow addition of a solution of 1.6 N n-butyllithium (35 mL, 56 mmol). After 30 minutes stirring, diethyloxalate (7.6 mL, 56 mmol) was added all at once. The mixture was slowly warmed to 0° C. before being quenched with NH₄Cl (aqueous) and being partitioned between water and ether. The ether was separated and washed with brine. Drying (MgSO₄) and removal of solvent gave 8.3 g of an oil, ¹H NMR (300 MHz, CHLOROFORM-D) δ ppm 1.34 (t, J=7.1 Hz, 3H) 4.36 (q, J=7.1 Hz, 2H) 7.02 (d, J=5.2 Hz, 1H) 7.66 (d, J=5.2 Hz, 1H).

Step 2. Preparation of ethyl (3-chlorothien-2-yl)(methoxyimino)acetate: A mixture of ethyl (3-chlorothien-2-yl)(oxo)acetate (3.0 g, 13.7 mmol), sodium acetate (1.7 g, 20.7 mmol) and methoxylamine hydrochloride (2.3 g, 27.6 mmol) in 30 mL ethanol was heated at 85° C. for 3 hours. The mixture was diluted with EtOAc and washed with water and brine. Drying (MgSO₄) and removal of solvent gave 3.6 g product as a mixture of geometrical isomers as an oil, ¹H NMR (300 MHz, MeOH-D4) δ ppm 1.33 (m, 3H) 3.96 and 4.09 (2s, 3H, 2:3 ratio) 4.23-4.47 (m, 2H) 6.87-7.10 (m, 1H) 7.6 and 7.7 (2d, 1H, 2:3 ratio).

Step 3. Preparation of 1-(3-chlorothien-2-yl)-2-ethoxy-2-oxoethanaminium trifluoroacetate: Aluminum foil (960 mg) was cut into small pieces and placed in 10 mL THF. A solution of HgCl₂ (300 mg) in 1 mL water was added. The mixture was stirred 5 minutes at room temperature. Solvent was removed via a pipette and the aluminum pieces were transferred to a solution of ethyl (3-chlorothien-2-yl)(oxo)acetate (3 g, 15 mmol) dissolved in 60 mL THF and 6 mL water. The mixture heated up, so an ice bath was applied to maintain the temperature around 30° C. After stirring for 2 hours, the mixture was filtered through Celite and rinsed through with EtOAc. Solvent was removed from the filtrate, and the residue was dissolved in methanol. Trifluoroacetic acid (1.2 mL) was added and solvent was removed. Trituration of the residue with ether gave 3.2 g of product as a white solid, ¹H NMR (300 MHz, DMSO-D6) δ ppm 1.18 (t, J=7.06 Hz, 3H) 4.24 (q, J=7.03 Hz, 2H) 5.61 (s, 1H) 7.19 (d, J=5.27 Hz, 1H) 7.86 (d, J=5.27 Hz, 1H) 8.87 (s, 3H).

Step 4. Preparation of 3-(3-chlorothien-2-yl)-5-(2-furyl)-1,3-dihydro-2H-pyrido[2,3-e][1,4]diazepin-2-one: Following the procedure of Step 2 of Example 6, 1-(3-chlorothien-2-yl)-2-ethoxy-2-oxoethanaminium trifluoroacetate (725 mg, 2.2 mmol) and 3-[2-furyl(imino)methyl]pyridin-2-amine (400 mg, 2.2 mmol) was converted to 320 mg of product as a white solid, ¹H NMR (300 MHz, DMSO-D6) δ ppm 5.03 (s, 1H) 6.71 (dd, J=3.58, 1.70 Hz, 1H) 6.87 (d, J=3.58 Hz, 1H) 7.10 (d, J=5.27 Hz, 1H) 7.39 (dd, J=7.91, 4.71 Hz, 1H) 7.70 (d, J=5.27 Hz, 1H) 7.99 (d, J=1.13 Hz, 1H) 8.18 (dd, J=7.82, 1.60 Hz, 1H) 8.70 (dd, J=4.71, 1.70 Hz, 1H) 11.39 (s, 1H).

Step 5. Preparation of 3-(3-chlorothien-2-yl)-5-(2-furyl)-N-methyl-3H-pyrido[2,3-e][1,4]diazepin-2-amine: Following the procedure of Step 3 of Example 1, 3-(3-chlorothien-2-yl)-5-(2-furyl)-1,3-dihydro-2H-pyrido[2,3-e][1,4]diazepin-2-one (100 mg, 0.29 mmol) was converted to the title product, ¹H NMR (300 MHz, DMSO-D6) δ ppm 2.70 (d, J=4.52 Hz, 3H) 4.62 (s, 1H) 6.69 (m, 2H) 6.84 (d, J=3.39 Hz, 1H) 7.07 (dd, J=7.82, 4.62 Hz, 1H) 7.17 (d, J=5.46 Hz, 1H) 7.78 (d, J=5.27 Hz, 1H) 7.89-8.01 (m, 2H) 8.61 (dd, J=4.52, 1.88 Hz, 1H).

### Example 17

### Preparation of 3-(4-bromothien-2-yl)-5-(4-fluorophenyl)-N-methyl-3H-pyrido[2,3-e][1,4]diazepin-2-amine

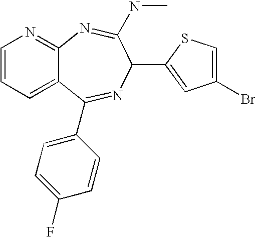

Step 1. Preparation of 3-[(4-fluorophenyl)(imino)methyl]pyridin-2-amine: A solution of 2M 4-fluoropenyl magnesium bromide (16.8 mL, 34 mmol) and 2-aminonicotinonitrile (0.5 g, 8.4 mmol) in 20 mL of THF was stirred at room temperature under nitrogen atmosphere for 18 hours. The reaction mixture was cooled in ice then quenched with ammonium chloride (aqueous) followed by water and EtOAc partitioning. The EtOAc was separated and dried with MgSO₄. Removal of solvent resulted in a yellow oil which was purified on flash column to yield a yellow solid (0.82 g, 45%). ¹H NMR (300 MHz, DMSO-D6) δ ppm 6.50 (dd, J=7.63, 4.80 Hz, 1H) 7.21 (dd, J=7.72, 1.32 Hz, 1H) 7.26-7.41 (m, 4H) 7.95-8.10 (m, 3H) 10.60 (s, 1H).

Step 2. Preparation of amino(4-bromothien-2-yl)acetonitrile: Ammonium chloride (2.8 g, 52 mmol) and potassium cyamide (1.9 g, 29 mmol) were dissolved in 40 mL of ammonia (aqueous). To this was added a solution of 4-bromothiophene-2-carboxaldehyde in 40 mL of diethyl ether. The bi-phasic mixture was sealed in a tube and heated to 60° for 6 hours. The reaction mixture was cooled to room temperature and the ether layer was separated. The aqueous layer was extracted with ether (50 mL×3). The combined ether extracts were dried with MgSO₄ and concentrated to a brown solid (5.54 g, 98%). ¹H NMR (300 MHz, DMSO-D6) δ ppm 3.12 (s, 2H) 5.31 (s, 1H) 7.14 (t, J=1.32 Hz, 1H) 7.67 (d, J=1.51 Hz, 1H).

Step 3. Preparation of methyl amino(4-bromothien-2-yl)acetate hydrochloride: A solution of amino(4-bromothien-2-yl)acetonitrile (7.54 g, 35 mmol) in 50 mL of methanol was added to a solution of thionyl chloride (7.6 mL, 104 mmol) in 100 mL of methanol. The reaction tube was sealed and heated overnight at an external temperature of 900. The reaction was cooled to room temperature and solvent was removed. The reaction mixture was then partitioned with 1N HCl and EtOAc. The aqueous portion was basified to pH 10 with Na₂CO₃ and extracted with EtOAc (50 mL×3). The organic portion was dried with MgSO₄ and concentrated to an orange oil. Purification on silica gel flash column (CH₂Cl₂ with gradient elution to 15% EtOAc in CH₂Cl₂) resulted in an orange solid (3.0 g, 34%). ¹H NMR (300 MHz, DMSO-D6) δ ppm 2.51 (s, 2H) 3.69 (s, 3H) 4.80 (s, 1H) 7.03 (s, 1H) 7.56 (s, 1H). A solution of methyl amino(4-bromothien-2-yl)acetate in 50 mL of methanol was added to was added to a solution of acetyl chloride (0.9 mL, 35 mmol) in 100 mL of methanol. The reaction was stirred for 10 minutes at room temperature and then concentrated to an orange solid (3.15 g, 92%). ¹H NMR (300 MHz, DMSO-D6) δ ppm 3.75 (s, 3H) 5.67 (s, 1H) 7.35 (s, 1H) 7.79 (s, 1H) 9.27 (s, 2H).

Step 4. Preparation of 3-(4-bromothien-2-yl)-5-(4-fluorophenyl)-1,3-dihydro-2H-pyrido[2,3-e][1,4]diazepin-2-one: A solution of 3-[(4-fluorophenyl)(imino)methyl]pyridin-2-amine (0.57 g, 2.6 mmol) and methyl amino(4-bromothien-2-yl)acetate hydrochloride (0.66 g, 2.6 mmol) in ethanol (5 mL, 200 proof) was heated in a microwave reactor for 30 minutes at 90°. DBU (1.04 mL, 7.8 mmol) was added via syringe and the solution was heated for 30 minutes at 100°. The reaction mixture was diluted with EtOAc and Na₂CO₃. The product was extracted with EtOAc (50 mL×3). Drying with MgSO₄ and removal of solvent gave an oily brown solid, which was recrystallized from methanol to give a light brown solid (0.89 g, 82%). ¹H NMR (300 MHz, DMSO-D6) δ ppm 5.22 (s, 1H) 7.13 (s, 1H) 7.27-7.41 (m, 3H) 7.61 (dd, J=8.67, 5.65 Hz, 2H) 7.67 (s, 1H) 7.86 (d, J=7.54 Hz, 1H) 8.70 (d, J=3.01 Hz, 1H) 11.31 (s, 1H).

Step 5. Preparation of 3-(4-bromothien-2-yl)-5-(4-fluorophenyl)-N-methyl-3H-pyrido[2,3-e][1,4]diazepin-2-amine: Following the procedure of Step 3 of Example 1, 3-(4-bromothien-2-yl)-5-(4-fluorophenyl)-1,3-dihydro-2H-pyrido[2,3-e][1,4]diazepin-2-one (0.10 g, 0.24 mmol) was converted to product. Purification was accomplished by dissolving product in CH₃CN and freeze-drying to yield a brown solid (0.085 g, 83%), ¹H NMR (300 MHz, CHLOROFORM-D) δ ppm 2.96 (d, J=4.90 Hz, 3H) 4.81 (s, 1H) 6.97 (dd, J=7.82, 4.62 Hz, 1H) 7.04-7.17 (m, 3H) 7.37 (d, J=1.32 Hz, 1H) 7.59-7.71 (m, 3H) 8.70 (dd, J=4.71, 1.88 Hz, 1H).

### Example 18

### 3-(4-bromothien-2-yl)-N-methyl-5-thien-2-yl-3H-pyrido[2,3-e][1,4]diazepin-2-amine

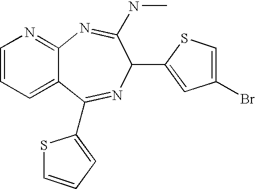

Step 1. Preparation of 3-(4-bromothien-2-yl)-5-thien-2-yl-1,3-dihydro-2H-pyrido[2,3-e][1,4]diazepin-2-one: Following the procedure of Step 4 of Example 17, 3-[imino(thien-2-yl)methyl]pyridin-2-amine (1.18 g, 5.8 mmol) and methyl amino(4-bromothien-2-yl)acetate hydrochloride (1.45 g, 5.8 mmol) were converted to product. No work-up necessary since product precipitated and was filtered and washed with ethanol to yield pure product (1.6 g, 70%). ¹H NMR (300 MHz, DMSO-D6) δ ppm 5.24 (s, 1H) 7.09 (s, 1H) 7.17 (d, J=3.01 Hz, 2H) 7.38 (dd, J=8.29, 4.52 Hz, 2H) 7.78-7.91 (m, 1H) 8.21 (d, J=8.29 Hz, 1H) 8.70 (d, J=3.01 Hz, 1H) 11.28 (s, 1H)

Step 2. Preparation of 3-(4-bromothien-2-yl)-N-methyl-5-thien-2-yl-3H-pyrido[2,3-e][1,4]diazepin-2-amine: Following the procedure of Step 3 of Example 1, 3-(4-bromothien-2-yl)-5-thien-2-yl-1,3-dihydro-2H-pyrido[2,3-e][1,4]diazepin-2-one (0.5 g, 1.2 mmol) was converted to the title compound (0.51 g, 100%). ¹H NMR (300 MHz, DMSO-D6) δ ppm 2.71 (d, J=4.52 Hz, 3H) 4.82 (s, 1H) 6.63 (d, J=4.90 Hz, 1H) 7.07 (dd, J=7.72, 4.71 Hz, 1H) 7.12-7.23 (m, 3H) 7.70-7.82 (m, 2H) 8.04 (dd, J=7.91, 1.88 Hz, 1H) 8.61 (dd, J=4.52, 1.88 Hz, 1H).

### Example 19

### 5-[2-(methylamino)-5-thien-2-yl-3H-pyrido[2,3-e][1,4]diazepin-3-yl]thiophene-3-carbonitrile

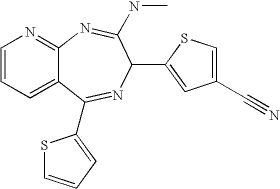

A mixture of 3-(4-bromothien-2-yl)-N-methyl-5-thien-2-yl-3H-pyrido[2,3-e][1,4]diazepin-2-amine (0.134 g, 0.3 mmol), zinc cyamide (0.023 g, 0.2 mmol), tris(dibenzylideneacetone)dipalladium(0) (0.01 g, 0.02 mmol), and 1,1′-bis(diphenylphosphino)ferrocene (0.017 g, 0.03 mmol) were sealed in a tube. DMF (1.5 mL) was added via syringe. The reaction was heated at 130° C. for 45 minutes. The reaction mixture was filtered through Celite and washed with EtOAc. It was then partitioned with water and EtOAc. The organic portion was dried with MgSO₄ and concentrated to a black oil which was deposited on a silica gel flash column (CH₂Cl₂ gradient elution to 20% MeOH in CH₂Cl₂). Purification yielded a brown solid which, when triturated with ether yields a pure white solid (0.055 g, 63%)ₘ, ¹H NMR (300 MHz, DMSO-D6) δ ppm 2.70 (d, J=4.52 Hz, 3H) 4.87 (s, 1H) 6.68 (d, J=4.52 Hz, 1H) 7.08 (dd, J=7.91, 4.52 Hz, 1H) 7.13-7.25 (m, 2H) 7.55 (s, 1H) 7.80 (d, J=4.90 Hz, 1H) 8.04 (dd, J=7.91, 1.88 Hz, 1H) 8.60-8.69 (m, 2H).

### Example 20

### 3-(4-bromothien-2-yl)-5-(2-furyl)-N-methyl-3H-pyrido[2,3-e][1,4]diazepin-2-amine

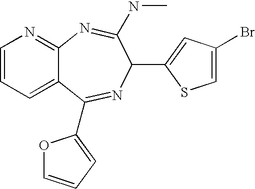

Step 1. Preparation of 3-(4-bromothien-2-yl)-5-(2-furyl)-1,3-dihydro-2-H-pyrido[2,3-e][1,4]diazepin-2-one: Following the procedure of Step 2 of Example 6, 3-[2-furyl(imino)methyl]pyridin-2-amine (1.0 g, 5.3 mmol) and methyl amino(4-bromothien-2-yl)acetate hydrochloride (1.32 g, 5.3 mmol) were converted to product. No work up necessary since product precipitated and was filtered and washed with methanol to yield pure product (0.60 g, 30%). ¹H NMR (300 MHz, DMSO-D6) δ ppm 5.23 (s, 1H) 6.70 (s, 1H) 6.85 (d, J=3.77 Hz, 1H) 7.11 (s, 1H) 7.36 (dd, J=7.54, 4.52 Hz, 1H) 7.67 (s, 1H) 7.98 (s, 1H) 8.17 (d, J=9.80 Hz, 1H) 8.69 (d, J=3.01 Hz, 1H) 11.28 (s, 1H).

Step 2. Preparation of 3-(4-bromothien-2-yl)-5-(2-furyl)-N-methyl-3H-pyrido[2,3-e][1,4]diazepin-2-amine: Following the procedure of Step 3 of Example 1, 3-(4-bromothien-2-yl)-5-(2-furyl)-1,3-dihydro-2H-pyrido[2,3-e][1,4]diazepin-2-one (0.20 g, 0.5 mmol) was converted to product (0.12 g, 60%). ¹H NMR (300 MHz, DMSO-D6) δ ppm 2.70 (d, J=4.52 Hz, 3H) 4.80 (s, 1H) 6.62 (d, J=4.52 Hz, 1H) 6.69 (s, 1H) 6.84 (d, J=3.77 Hz, 1H) 7.06 (dd, J=7.91, 4.90 Hz, 1H) 7.22 (s, 1H) 7.75 (s, 1H) 7.92-8.05 (m, 2H) 8.61 (s, 1H).

### Example 21

### 3-(2-furyl)-N-methyl-5-thien-2-yl-3H-pyrido[2,3-e][1,4]diazepin-2-amine

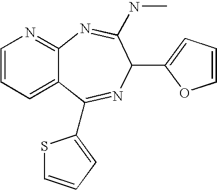

Step 1. Preparation of methyl 2-furyl(methoxyimino)acetate: A solution of ammonium 2-furyl(methoxyimino)acetate (5.0 g, 29 mmol) in 100 mL of methanol was added to a solution of acetyl chloride (3.1 mL, 44 mmol) in 50 mL of methanol and heated to reflux overnight. The solvent was removed and the residue was partitioned between EtOAc and water. Drying (MgSO₄) and removal of solvent resulted in an orange oil (4.4 g, 85%). Product is a 3:1 mixture of geometric isomers. ¹H NMR (300 MHz, DMSO-D6) δ ppm 3.86, 3.88 (2s, 3H, 3:1) 4.06, 3.93 (2s, 3H, 3:1) 6.73, 6.66 (2 dd, J=3.49, 1.79 Hz, 1H, 3:1) 7.32, 6.86 (2d, J=3.39 Hz, 1H, 3:1) 7.87-7.93 (2m, 1H, 3:1).

Step 2. Preparation of methyl amino(2-furyl)acetate hydrochloride: A 50% solution of formic acid in water (60 mL) was added to a solution of methyl 2-furyl(methoxyimino)acetate (4.4 g, 24 mmol) in 100 mL of methanol. The reaction was cooled to 0° and zinc dust (4.7 g, 72 mmol) was added. The reaction was warmed to room temperature and stirred over night. The reaction mixture was filtered through Celite and washed with methanol. The solvent was removed from the filtrate and the residue was dissolved in water. The aqueous portion was basified to pH 10 with Na₂CO₃ and the product was extracted with EtOAc. Drying (MgSO₄) and removal of solid resulted in methyl amino(2-furyl)acetate as a yellow oil (2.9 g, 78%). ¹H NMR (300 MHz, DMSO-D6) δ ppm 2.21 (s, 2H) 3.64 (s, 3H) 4.58 (s, 1H) 6.33 (d, J=3.20 Hz, 1H) 6.40 (dd, J=3.01, 1.88 Hz, 1H) 7.53-7.59 (m, 1H). A solution of methyl amino(2-furyl)acetate in 50 mL of methanol was added to a solution of acetyl chloride (1.3 mL, 19 mmol) in 100 mL of methanol. After stirring for 10 minutes the solution was concentrated to a yellow solid (1.64 g). ¹H NMR (300 MHz, DMSO-D6) δ ppm 3.76 (s, 3H) 5.54 (s, 1H) 6.55 (dd, J=3.30, 1.79 Hz, 1H) 6.70 (d, J=3.39 Hz, 1H) 7.73-7.83 (m, 1H) 9.14 (s, 2H).

Step 3. Preparation of 3-(2-furyl)-5-thien-2-yl-1,3-dihydro-2H-pyrido[2,3-e][1,4]diazepin-2-one: Following the procedure of Step 2 of Example 6, 3-[imino(thien-2-yl)methyl]pyridin-2-amine (0.3 g, 1.4 mmol) and methyl amino(2-furyl)acetate hydrochloride (0.28 g, 1.4 mmol) were converted to product and trituration with ether yielded pure product as an orange solid (0.20 g, 47%). ¹H NMR (300 MHz, DMSO-D6) δ ppm 4.99 (s, 1H) 6.51 (d, J=1.70 Hz, 1H) 6.59 (d, J=3.01 Hz, 1H) 7.07-7.19 (m, 2H) 7.36 (dd, J=7.72, 4.71 Hz, 1H) 7.71 (s, 1H) 7.81 (dd, J=4.52, 1.70 Hz, 1H) 8.18 (dd, J=7.82, 1.41 Hz, 1H) 8.68 (dd, J=4.62, 1.41 Hz, 1H) 11.21 (s, 1H).

Step 4. Preparation of 3-(2-furyl)-N-methyl-5-thien-2-yl-3H-pyrido[2,3-e][1,4]diazepin-2-amine: Following the procedure of Step 3 of Example 1, 3-(2-furyl)-5-thien-2-yl-1,3-dihydro-2H-pyrido[2,3-e][1,4]diazepin-2-one (0.15 g, 0.5 mmol) was converted to crude product. The crude product was purified by trituration with ether to yield a pure, yellow solid (0.069 g, 43%). ¹H NMR (300 MHz, DMSO-D6) δ ppm 2.72 (d, J=4.52 Hz, 3H) 4.56 (s, 1H) 6.50 (m, 1H) 6.59 (dd, J=3.20, 1.88 Hz, 1H) 6.75 (d, J=3.20 Hz, 1H) 7.07 (dd, J=7.82, 4.62 Hz, 1H) 7.12-7.18 (m, 2H) 7.70-7.80 (m, 2H) 8.02 (dd, J=7.82, 1.98 Hz, 1H) 8.60 (dd, J=4.62, 1.98 Hz, 1H).

### Example 22

### 3,5-di-2-furyl-N-methyl-3H-pyrido[2,3-e][1,4]diazepin-2-amine

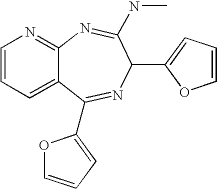

Step 1. Preparation of 3,5-di-2-furyl-1,3-dihydro-2H-pyrido[2,3-e][1,4]diazepin-2-one: Following the procedure of Step 2 of Example 6, 3-[2-furyl(imino)methyl]pyridin-2-amine (0.30 g, 1.6 mmol) and methyl amino(2-furyl)acetate hydrochloride (0.31 g, 1.6 mmol) were converted to product and purified by trituration with ether to yield pure product (0.20 g, 43%). ¹H NMR (300 MHz, DMSO-D6) δ ppm 4.98 (s, 1H) 6.49 (s, 1H) 6.60 (d, J=2.83 Hz, 1H) 6.69 (dd, J=3.49, 1.79 Hz, 1H) 6.85 (d, J=3.39 Hz, 1H) 7.35 (dd, J=7.91, 4.71 Hz, 1H) 7.71 (s, 1H) 7.94 (d, J=1.13 Hz, 1H) 8.14 (dd, J=7.82, 1.41 Hz, 1H) 8.67 (dd, J=4.62, 1.60 Hz, 1H) 11.22 (s, 1H).

Step 2. Preparation of 3,5-di-2-furyl-N-methyl-3H-pyrido[2,3-e][1,4]diazepin-2-amine: Following the procedure of Step 3 of Example 1, 3,5-di-2-furyl-1,3-dihydro-2H-pyrido[2,3e][1,4]diazepin-2-one (0.15 g, 0.51 mmol) was converted to the title compound (0.015 g). ¹H NMR (300 MHz, DMSO-D6) δ ppm 2.71 (d, J=4.71 Hz, 3H) 4.54 (s, 1H) 6.49 (d, J=4.52 Hz, 1H) 6.59 (dd, J=3.11, 1.79 Hz, 1H) 6.67 (dd, J=3.39, 1.70 Hz, 1H) 6.77 (d, J=3.20 Hz, 1H) 6.84 (d, J=3.20 Hz, 1H) 7.06 (dd, J=7.82, 4.62 Hz, 1H) 7.78 (d, J=0.94 Hz, 1H) 7.87-7.99 (m, 2H) 8.59 (dd, J=4.52, 1.88 Hz, 1H).

### Example 23

### 5-(2-furyl)-3-(4-iodothien-2-yl)-N-methyl-3H-pyrido[2,3-e][1,4]diazepin-2-amine

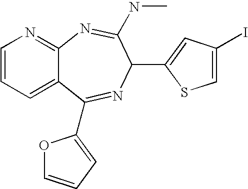

Step 1. Preparation of 2-(4-bromothien-2-yl)-1,3-dioxolane: A solution of 4-bromothiophene-2-carboxaldehyde (15.0 g, 79 mmol), ethylene glycol (5.2 mL, 94 mmol), and catalytic amount of p-toluenesulfonic acid (0.05 g) in anhydrous benzene was heated to reflux with azeotropic removal of water. After heating overnight, the reaction was cooled to room temperature and the benzene was removed. The residue was partitioned between EtOAc and water. The EtOAc was separated dried with MgSO₄, and concentrated to afford a black oil. Distillation yielded a colorless oil (15.4 g, 85%) ¹H NMR (300 MHz, DMSO-D6) δ ppm 3.911-4.05 (m, 4H) 6.04 (s, 1H) 7.25 (d, J=1.13 Hz, 1H) 7.72 (d, J=1.51 Hz, 1H).

Step 2. Preparation of 4-iodothiophene-2-carbaldehyde: A solution of 2-(4-bromothien-2-yl)-1,3-dioxolane (8.02 g, 34 mmol) in ether (150 mL, anhydrous) was cooled to −78° followed by the dropwise addition of 2.5 M n-BuLi in hexanes (16.3 mL, 41 mmol). The reaction was stirred for 20 minutes. A solution of iodine (8.64 g, 34 nmol) in ether (50 mL, anhydrous) was added dropwise resulting in a red solution upon addition of the last drop. The reaction was warmed to room temperature and stirred for 20 minutes. 1N HCl (100 mL) was added and the reaction was stirred overnight. The reaction mixture was diluted with EtOAc and partitioned with water. Drying (MgSO₄) and removal of solvent gave a yellow oil (7.0 g, 87%). ¹H NMR (300 MHz, DMSO-D6) δ ppm 8.10 (d, J=1.32 Hz, 1H) 8.33 (t, J=1.22 Hz, 1H) 9.90 (d, J=1.32 Hz, 1H).

Step 3. Preparation of amino(4-iodothien-2-yl)acetonitrile: Following the procedure of Step 2 of Example 17, 4-iodothiophene-2-carbaldehyde (7.0 g, 29 mmol) was converted to product (7.2 g, 95%). ¹H NMR (300 MHz, DMSO-D6) δ ppm 3.09 (s, 2H) 5.32 (s, 1H) 7.15 (s, 1H) 7.75 (d, J=1.13 Hz, 1H).

Step 4. Preparation of methyl amino(4-iodothien-2-yl)acetate hydrochloride: Following the procedure of Step 3 of Example 17, amino(4-iodothien-2-yl)acetonitrile (7.2 g, 27 mmol) was converted to methyl amino(4-iodothien-2-yl)acetate (2.99 g). ¹H NMR (300 MHz, DMSO-D6) δ ppm 3.77 (s, 3H) 5.71 (s, 1H) 7.36 (d, J=1.32 Hz, 1H) 7.89 (d, J=1.51 Hz, 1H) 9.16 (s, 2H) Methyl amino(4-iodothien-2-yl)acetate (2.99 g) was converted to methyl amino(4-iodothien-2-yl)acetate hydrochloride (3.14 g, 35%).

Step 5. Preparation of 5-(2-furyl)-3-(4-iodothien-2-yl)-1,3-dihydro-2H-pyrido[2,3-e][1,4]diazepin-2-one: Following the procedure of Step 2 of Example 6, 3-[2-furyl(imino)methyl]pyridin-2-amine (0.84 g, 4.5 mmol) and methyl amino(4-iodothien-2-yl)acetate hydrochloride (1.5 g, 4.5 mmol) were converted to product which precipitated from the reaction mixture as a white solid (1.04 g, 53%) ¹H NMR (300 MHz, DMSO-D6) δ ppm 5.24 (s, 1H) 6.63-6.77 (m, 1H) 6.84 (d, J=3.39 Hz, 1H) 7.13 (s, 1H) 7.36 (dd, J=7.44, 4.99 Hz, 1H) 7.76 (s, 1H) 7.97 (s, 1H) 8.17 (d, J=7.91 Hz, 1H) 8.69 (d, J=4.71 Hz, 1H) 11.27 (s, 1H).

Step 6. Preparation of 5-(2-furyl)-3-(4-iodothien-2-yl)-N-methyl-3H-pyrido[2,3-e][1,4]diazepin-2-amine: Following the procedure of Step 3 of Example 1, 5-(2-furyl)-3-(4-iodothien-2-yl)-1,3-dihydro-2H-pyrido[2,3-e][1,4]diazepin-2-one (1.04 g, 2.4 mmol) was converted to the title compound and triturated to a white solid (0.70 g, 65%). ¹H NMR (300 MHz, DMSO-D6) δ ppm 2.69 (d, J=4.52 Hz, 3H) 4.80 (s, 1H) 6.57 (d, J=4.52 Hz, 1H) 6.68 (dd, J=3.39, 1.70 Hz, 1H) 6.83 (d, J=3.39 Hz, 1H) 7.05 (dd, J=7.72, 4.52 Hz, 1H) 7.24 (s, 1H) 7.83 (d, J=11.13 Hz, 1H) 7.94 (s, 1H) 7.97 (dd, J=7.72, 1.88 Hz, 1H) 8.60 (dd, J=4.52, 1.88 Hz, 1H).

### Example 24

### 3-{5-[5-(2-furyl)-2-(methylamino)-3H-pyrido[2,3-e][1,4]diazepin-3-yl]thien-3-yl}prop-2-yn-1-ol

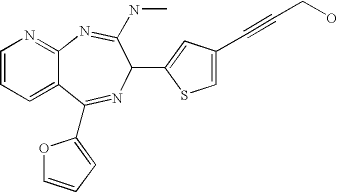

A mixture of 5-(2-furyl)-3-(4-iodothien-2-yl)-N-methyl-3H-pyrido[2,3-e][1,4]diazepin-2-amine (0.10 g, 0.2 mmol), copper iodide (0.008 g, 0.04 mmol) and tetrakis(triphenyl phosphine)palladium (0) (0.023 g, 0.02 mmol) were sealed in a microwave tube. The system was flushed with argon. To this was added a solution of triethylamine (anhydrous):acetonitrile (2.5:1). The reaction mixture was freeze-pump-thawed (3 times). The acetylene was added to the reaction via syringe. The tube was heated in the microwave at 100° for 30 minutes. The reaction mixture was partitioned with water and EtOAc. Drying with MgSO₄ and removal of solvent gave a black oil. Normal phase purification on silica gel (CH₂Cl₂ gradient elution to 10% MeOH in CH₂Cl₂) yielded impure product. Further purification by reverse-phase HPLC (20-50% gradient of CH₃CN in water with 0.1% TFA) resulted in pure product. The product was free-based using EtOAc and NaHCO₃ partitioning. Drying with MgSO₄, removal of solvent followed by trituration with ether resulted in 15 mg of pure product. ¹H NMR (300 MHz, DMSO-D6) δ ppm 2.70 (d, J=4.14 Hz, 3H) 4.28 (d, J=5.65 Hz, 2H) 4.80 (s, 1H) 5.32 (t, J=5.84 Hz, 1H) 6.60 (d, J=4.33 Hz, 1H) 6.68 (s, 1H) 6.84 (d, J=3.20 Hz, 1H) 7.07 (dd, J=7.63, 4.80 Hz, 1H) 7.20 (s, 1H) 7.80 (s, 1H) 7.94 (s, 1H) 7.99 (d, J=7.72 Hz, 1H) 8.60 (d, J=2.83 Hz, 1H).

### Example 25

### 3-{4-[3-(dimethylamino)prop-1-ynyl]thien-2-yl}-5-(2-furyl)-N-methyl-3H-pyrido[2,3-e][1,4]diazepin-2-amine

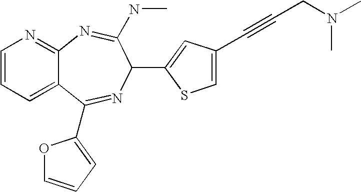

Following the procedure of Example 24, 5-(2-furyl)-3-(4-iodothien-2-yl)-N-methyl-3H-pyrido[2,3-e][1,4]diazepin-2-amine (0.10 g, 0.2 mmol) and N,N-dimethyl-N-prop-2-ynylamine (0.034 g, 0.3 mmol) were converted to product (15 mg). Additional purification on normal phase was not necessary. ¹H NMR (300 MHz, DMSO-D6) δ ppm 2.26 (s, 6H) 2.70 (d, J=4.52 Hz, 3H) 3.47 (s, 2H) 4.78 (s, 1H) 6.57 (d, J=4.52 Hz, 1H) 6.68 (dd, J=3.39, 1.88 Hz, 1H) 6.84 (d, J=3.39 Hz, 1H) 7.06 (dd, J=7.82, 4.62 Hz, 1H) 7.21 (s, 1H) 7.80 (d, J=1.32 Hz, 1H) 7.94 (d, J=1.13 Hz, 2H) 8.60 (dd, J=4.62, 1.98 Hz, 1H).

### Example 26

### 4-{5-[5-(2-furyl)-2-(methylamino)-3H-pyrido[2,3-e][1,4]diazepin-3-yl]thien-3-yl}-2-methylbut-3-yn-2-ol

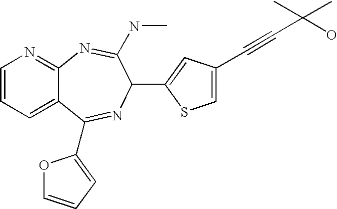

Following the procedure of Example 24, 5-(2-furyl)-3-(4-iodothien-2-yl)-N-methyl-3H-pyrido[2,3-e][1,4]diazepin-2-amine (0.20 g, 0.5 mmol) and 2-methylbut-3-yn-2-ol (0.065 g, 0.68 mmol) were converted to product which was purified by normal phase on silica gel (CH₂Cl₂ gradient elution to 5% MeOH in CH₂Cl₂) resulting in pure product (50 mg). ¹H NMR (300 MHz, DMSO-D6) δ ppm 1.45 (s, 6H) 3.34 (s, 3H) 4.87 (d, J=19.59 Hz, 1H) 5.45 (s, 1H) 6.69 (s, 2H) 6.85 (d, J=2.26 Hz, 1H) 7.16 (m, 2H) 7.74 (s, 1H) 7.95 (s, 1H) 8.05 (m, 1H).

### Example 27

### 4-{5-[5-(2-furyl)-2-(methylamino)-3H-pyrido[2,3-e][1,4]diazepin-3-yl]thien-3-yl}but-3-yn-2-ol

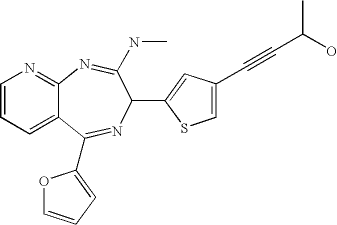

Following the procedure of Example 24, 5-(2-furyl)-3-(4-iodothien-2-yl)-N-methyl-3H-pyrido[2,3-e][1,4]diazepin-2-amine (0.15 g, 0.3 mmol) and but-3-yn-2-ol (0.038 mL, 0.5 mmol) were converted to product, which was purified by triturating with CH₂Cl₂ to yield pure product (18 mg). ¹H NMR (300 MHz, DMSO-D6) δ ppm 1.37 (d, J=6.59 Hz, 3H) 3.33 (s, 3H) 4.58 (m, 1H) 5.45 (2s, J=5.27 Hz, 1H) 6.69 (s, 2H) 6.85 (m, 2H) 7.23 (m, 1H) 7.81 (m, 2H) 7.97 (m, 2H).

### Example 28

### 4-{5-[5-(2-furyl)-2-(methylamino)-3H-pyrido[2,3-e][1,4]diazepin-3-yl]thien-3-yl}but-3-yn-1-ol

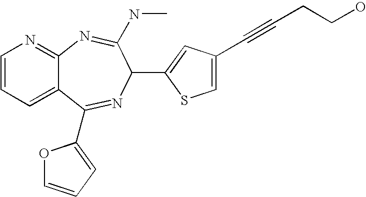

Following the procedure of Example 24, 5-(2-furyl)-3-(4-iodothien-2-yl)-N-methyl-3H-pyrido[2,3-e][1,4]diazepin-2-amine (0.15 g, 0.3 mmol) and but-3-yn-1-ol (0.038 mL, 0.5 mmol) were converted to product which was purified by normal phase (CH₂Cl₂ gradient elution to 5% MeOH in CH₂Cl₂) yielded pure product (15 mg). ¹H NMR (300 MHz, DMSO-D6) δ ppm 2.40 (t, J=6.50 Hz, 2H) 3.34 (s, 3H) 3.49 (t, J=6.03 Hz, 1H) 3.56 (dd, J=12.34, 6.59, 6.31 Hz, 2H) 4.90 (s, 1H) 6.68 (m, 2H) 6.83 (m, 2H) 7.20 (m, 1H) 7.74 (t, 2H) 7.87-8.00 (m, 2H).

### Example 29

### N-(3-{5-[5-(2-furyl)-2-(methylamino)-3H-pyrido[2,3-e][1,4]diazepin-3-yl]thien-3-yl}prop-2-ynyl)acetamide

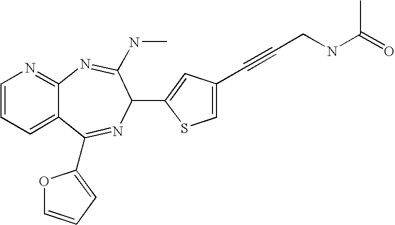

Following the procedure of Example 24, 5-(2-furyl)-3-(4-iodothien-2-yl)-N-methyl-3H-pyrido[2,3-e][1,4]diazepin-2-amine (0.13 g, 0.3 mmol) and N-prop-2-ynylacetamide (0.04 g, 0.4 mmol) were converted to product which was purified by normal phase on silica gel (CH₂Cl₂ gradient elution to 5% MeOH in CH₂Cl₂) resulted in pure product (5 mg). ¹H NMR (300 MHz, MeOD) δ ppm 1.92-1.97 (m, 3H) 2.85 (s, 3H) 4.09-4.17 (m, 2H) 4.85 (s, 1H) 6.61 (dd, J=3.39, 1.70 Hz, 1H) 6.88 (d, J=3.39 Hz, 1H) 7.12 (dd, J=7.35, 5.09 Hz, 1H) 7.18 (s, 1H) 7.60 (s, 1H) 7.73 (s, 1H) 8.05-8.15 (m, 1H) 8.57 (s, 1H).

### Example 30

### 5-(4-fluorophenyl)-N,8-dimethyl-3-phenyl-3H-pyrido[2,3-e][1,4]diazepin-2-amine

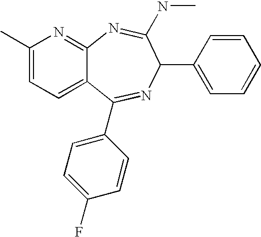

Step 1. Preparation of 3-[(4-fluorophenyl)(imino)methyl]-6-methylpyridin-2-amine: 2-amino-6-methylnicotinonitrile (1.36 g, 10.2 mmol) in 10 mL THF was stirred under N₂(g) while a 3-fold excess of 4-fluorophenylmagnesium bromide (15.3 mL of a 2M soln in diethylether) was added via syringe. The mixture was then heated at 40° C. until all starting material was consumed. The reaction was quenched with NH₄Cl (aq) and product extracted with EtOAc. The EtOAc extracts were washed with water, dried (MgSO₄), and concentrated to yield 2.8 g red/orange crude product, MS (M+H)=230.

Step 2. Preparation of 5-(4-fluorophenyl)-8-methyl-3-phenyl-1,3-dihydro-2H-pyrido[2,3-e][1,4]diazepin-2-one: Crude 3-[(4-fluorophenyl)(imino)methyl]-6-methylpyridin-2-amine (595 mg, 2.6 mmol), phenylglycine methylester (520 mg, 2.6 mmol), and 5 mL anhydrous EtOH was heated at 150° C. for 20 minutes in a microwave reactor [This reaction also goes to completion at 80° C. after 20 minutes and methanol can be substituted for EtOH]. The microwave vessel was then charged with DBU (1.2 mL) and heated at 150° C. for 15 mins in the microwave reactor [This step was also found to reach completion at 80° C. in 25 minutes]. The mixture was concentrated under vacuum to a thick liquid and then partitioned between NH₄Cl (aq) and EtOAc. The EtOAc fractions were collected, washed with water, dried (MgSO₄), and concentrated. Crude product was purified on flash silica to yield 240 mg brown product, MS (M+H)=346.

Step 3. Preparation of 5-(4-fluorophenyl)-N,8-dimethyl-3-phenyl-3H-pyrido[2,3-e][1,4]diazepin-2-amine: To a N₂(g) purged vessel containing 5-(4-fluorophenyl)-8-methyl-3-phenyl-1,3-dihydro-2H-pyrido[2,3-e][1,4]diazepin-2-one (240 mg) was added 5 mL of 2M N-methylamine in THF followed by 1 mL TiCl₄ (1M soln in CH₂Cl₂). The mixture was heated at 80° C. for 30 minutes in a microwave reactor. The suspension was then diluted with NH₄Cl (aq) and EtOAc, and filtered through Celite. The EtOAc layer was then concentrated and the crude product purified by reverse phase chromatography employing a mobile phase of CH₃CN with 0.1% TFA, MS (M+H)=359, ¹H NMR (300 MHz, DMSO-D6) δ ppm 2.65 (s, 3H) 2.78 (s, 3H) 4.98 (s, 1H) 7.21 (s, 1H) 7.31 (s, 2H) 7.47 (s, 4H) 7.67 (s, 4H) 8.13 (s, 1H).

### Example 31

### N-cyclopropyl-5-(4-fluorophenyl)-8-methyl-3-thien-2-yl-3H-pyrido[2,3-e][1,4]diazepin-2-amine

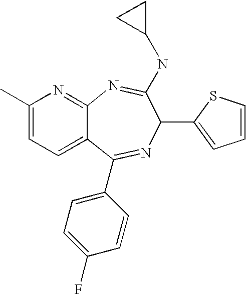

Step 1. Preparation of 5-(4-fluorophenyl)-8-methyl-3-(2-thienyl)-1,3-dihydro-2H-pyrido[2,3-e][1,4]diazepin-2-one: Prepared as in Example 30 Step 2 employing crude 3-[(4-fluorophenyl)(imino)methyl]-6-methylpyridin-2-amine (800 mg, 3.5 mmol) and methyl amino(thien-2-yl)acetate HCl salt (715 mg, 3.5 mmol). Crude product was purified by flash silica and then precipitated with MeOH to yield 240 mg product, MS (M+H)=352, ¹H NMR (300 MHz, DMSO-D6) δ ppm 2.56 (s, 3H) 5.14 (s, 1H) 7.02-7.12 (m, 2H) 7.16 (d, J=8.29 Hz, 1H) 7.30 (t, J=9.04 Hz, 2H) 7.50-7.65 (m, 2H) 7.71 (d, J=7.54 Hz, 2H) 11.10 (s, 1H).

Step 2. Preparation of N-cyclopropyl-5-(4-fluorophenyl)-8-methyl-3-thien-2-yl-3H-pyrido[2,3-e][1,4]diazepin-2-amine: Prepared as in Example 30 Step 3 employing 5-(4-fluorophenyl)-8-methyl-3-(2-thienyl)-1,3-dihydro-2H-pyrido[2,3-e][1,4]diazepin-2-one (120 mg), 3 eq TiCl₄, 175 μL of cyclopropylamine and 5 mL anhydrous THF to yield the title compound, MS (M+H)=391, ¹H NMR (300 MHz, DMSO-D6) δ ppm 0.41-0.54 (m, 1H) 0.56-0.71 (m, 3H) 2.64 (s, 3H) 2.91-3.06 (m, 1H) 5.15 (s, 1H) 7.11-7.23 (m, 2H) 7.25-7.37 (m, 3H) 7.57-7.71 (m, 3H) 8.09 (d, J=7.54 Hz, 1H).

### Example 32

### N,8-dimethyl-5-phenyl-3-thien-2-yl-3H-pyrido[2,3-e][1,4]diazepin-2-amine

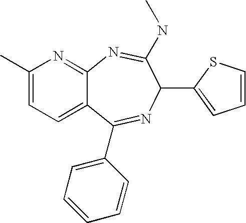

Step 1. Preparation of 3-[imino(phenyl)methyl]-6-methylpyridin-2-amine: Prepared as for Example 30 Step 1 employing 2-amino-6-methylnicotinonitrile (1.36 g, 10.2 mmol) and 3-fold excess of phenylmagnesium bromide (1M solution in diethylether). The reaction was stirred at 40° C. overnight and after workup yielded 2.27 g red liquid crude product, MS (M+H)=212.

Step 2. Preparation of 8-methyl-5-phenyl-3-thien-2-yl-1,3-dihydro-2H-pyrido[2,3-e][1,4]diazepin-2-one: Prepared as for Example 30 Step 2 employing crude 3-[imino(phenyl)methyl]-6-methylpyridin-2-amine (800 mg, 3.79 mmol) and methyl amino(thien-2-yl)acetate HCl salt (779 mg, 3.79 mmol). Crude material was purified on flash silica to yield 75 mg brown product, MS (M+H)=334.

Step 3. Preparation of N,8-dimethyl-5-phenyl-3-thien-2-yl-3H-pyrido[2,3-e][1,4]diazepin-2-amine: Prepared as for Example 30 Step 3 employing 8-methyl-5-phenyl-3-thien-2-yl-1,3-dihydro-2H-pyrido[2,3-e][1,4]diazepin-2-one (75 mg), 650 μL TiCl₄ (1M soln in CH₂Cl₂), and 5 mL N-methylamine (2M in THF) to give title compound MS (M+H)=347, ¹H NMR (300 MHz, DMSO-D6) δ ppm 2.70 (s, 3H) 2.85 (d, J=4.52 Hz, 3H) 5.30 (s, 1H) 7.23 (td, J=8.29, 3.77 Hz, 3H) 7.49-7.64 (m, 5H) 7.69 (d, J=6.03 Hz, 1H) 7.87 (d, J=4.52 Hz, 1H) 8.14 (d, J=7.54 Hz, 1H).

### Example 33

### 5-(4-fluorophenyl)-N,8-dimethyl-3-thien-2-yl-3H-pyrido[2,3-e][1,4]diazepin-2-amine

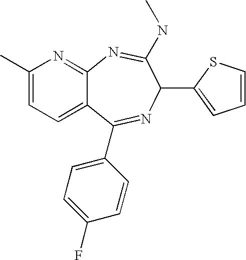

Prepared as in Example 30 Step 3 employing 5-(4-fluorophenyl)-8-methyl-3-(2-thienyl)-1,3-dihydro-2H-pyrido[2,3-e][1,4]diazepin-2-one (120 mg, see example 31), 3 eq TiCl₄, and 5 mL N-methylamine (2M soln in THF) to yield the title compound, MS (M+H)=365, ¹H NMR (300 MHz, DMSO-D6) δ ppm 2.61-2.67 (m, 3H) 2.79 (d, J=4.52 Hz, 3H) 5.23 (s, 1H) 7.14-7.25 (m, 3H) 7.26-7.39 (m, 2H) 7.58-7.72 (m, 3H) 7.81 (d, 1H) 8.10 (d, J=8.29 Hz, 1H).

### Example 34

### N²-cyclopropyl-5-(4-fluorophenyl)-3-thien-2-yl-3H-pyrido[2,3-e][1,4]diazepine-2,8-diamine

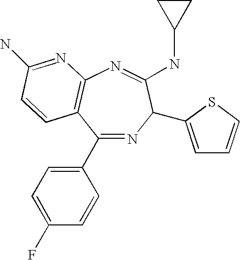

Step 1. Preparation of 2,6-difluoronicotinonitrile: 2,6-difluoropyridine in 50 mL anhydrous THF was added dropwise to a freshly prepared solution of lithium diisopropylamine (from 15.4 mL diisopropylamine and 1.1 eq n-BuLi) emerged in a dry ice/acetone bath. The combined mixture was stirred for 2 hours at −78° C. A second flask was charged with 20 grams tosylcyanate dissolved in 100 mL anhydrous THF and submerged in a dry ice/acetone bath. The first solution was transferred to the second via cannula and the combined mixture stirred at −78° C. for 40 mins before quenching with NH₄Cl (aq). Product was extracted from the aqueous layer with Et₂O and the combined organic extracts then washed with water, dried (MgSO₄), and concentrated. Product was isolated by flash silica purification, 4.1 g light brown liquid, ¹H NMR (300 MHz, DMSO-D6) δ ppm 7.57 (d, J=8.29 Hz, 1H) 8.81-8.91 (q, 1H).

Step 2. Preparation of 2,6-diaminonicotinonitrile: In a sealable container was combined 4.1 g 2,6-difluoronicotinonitrile and 40 mL concentrated ammonium hydroxide. The sealed vessel was then submerged in an 88° C. oil bath with stirring for 12 hours. Crystalline precipitate appeared upon cooling of the reaction mixture to room temperature. The solid was collected by filtration and rinsed with water to yield 2.5 g product, MS (M+H)=135, ¹H NMR (300 MHz, DMSO-D6) δ ppm 5.73 (d, J=8.29 Hz, 1H) 6.22 (s, 2H) 6.49 (s, 2H) 7.32 (d, J=8.29 Hz, 1H).

Step 3. Preparation of 3-[(4-fluorophenyl)(imino)methyl]pyridine-2,6-diamine: Prepared as for Example 30 Step 1 employing 2,6-diaminonicotinonitrile (1.0 g, 7.4 mmol) and 5-fold excess of 4-fluorophenylmagnesium bromide (2M soln in diethylether) and refluxing the mixture overnight to yield after aqueous workup 1.6 g crude brown product, MS (M+H)=231.

Step 4. Preparation of 8-amino-5-(4-fluorophenyl)-3-thien-2-yl-1,3-dihydro-2H-pyrido[2,3-e][1,4]diazepin-2-one: Prepared as for Example 30 Step 2 employing crude 3-[(4-fluorophenyl)(imino)methyl]pyridine-2,6-diamine (500 mg, 2.17 mmol) and methyl amino(thien-2-yl)acetate HCl salt (447 mg, 2.17 mmol). Pure product was precipitated from MeOH following aqueous workup and collected by filtration, 160 mg, MS (M+H)=353, ¹H NMR (300 MHz, DMSO-D6) δ ppm 4.83 (s, 1H) 6.07 (s, 1H) 6.61 (s, 2H) 6.84 (s, 2H) 7.08 (s, 3H) 7.25 (s, 1H) 7.35 (s, 2H) 10.39 (s, 1H).

Step 5. Preparation of N²-cyclopropyl-5-(4-fluorophenyl)-3-thien-2-yl-3H-pyrido[2,3-e][1,4]diazepine-2,8-diamine: Prepared as in Example 30 Step 3 employing 8-amino-5-(4-fluorophenyl)-3-thien-2-yl-1,3-dihydro-2H-pyrido[2,3-e][1,4]diazepin-2-one (70 mg), 3 eq TiCl₄, 5 mL anhydrous THF, and 175 μL cyclopropylamine to yield the title compound, MS (M+H)=392, ¹H NMR (300 MHz, DMSO-D6) δ ppm 0.46-0.55 (m, 1H) 0.58-0.72 (m, 3H) 2.93 (m, 1H) 5.03 (s, 1H) 6.44 (d, J=9.04 Hz, 1H) 7.15-7.25 (m, 2H) 7.35 (t, J=9.04 Hz, 2H) 7.49 (m, 1H) 7.61-7.74 (m, 3H).

### Example 35

### 5-(4-fluorophenyl)-N²-methyl-3-thien-2-yl-3H-pyrido[2,3-e][1,4]diazepine-2,8-diamine

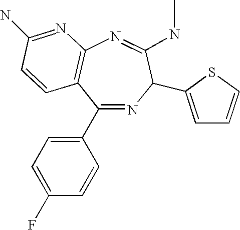

Prepared as in Example 30 Step 3 employing 8-amino-5-(4-fluorophenyl)-3-thien-2-yl-1,3-dihydro-2H-pyrido[2,3-e][1,4]diazepin-2-one (70 mg), 3 eq TiCl₄, and 5 mL N-methylamine (2M soln in THF) to yield the title compound, MS (M+H)=366, ¹H NMR (300 MHz, DMSO-D6) δ ppm 2.78 (d, J=4.52 Hz, 3H) 5.09 (s, 1H) 6.43 (d, J=9.04 Hz, 1H) 7.19-7.28 (m, 2H) 7.35 (t, J=8.67 Hz, 2H) 7.51 (m, 1H) 7.61-7.73 (m, 4H).

### Example 36

### 7-chloro-N-methyl-3,5-dithien-2-yl-3H-pyrido[2,3-e][1,4]diazepin-2-amine

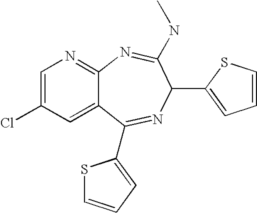

Step 1. Preparation of 2-amino-5-chloronicotinonitrile: Charged microwave vessel with 3-bromo-5-chloropyridin-2-amine (2.65 g, 0.013 mmol), Zn(CN)₂ (940 mg), DPPF (25 mg), and 5 mL wet DMF. The solution was then purged with N₂(g) prior to the addition of Pd₂ dba (20 mg). The reaction was heated at 120° C. for 30 mins in a microwave reactor yielding crystalline product. Product was collected by filtration and washed with both DMF and water to yield 1.6 g solid yellow product contaminated with ˜10% starting material. MS (M+H)=154.

Step 2. Preparation of 5-chloro-3-[imino(thien-2-yl)methyl]pyridin-2-amine: Prepared as for Example 30 Step 1 employing 2-amino-5-chloronicotinonitrile (1.0 g, 6.5 mmol) and 3-fold excess of thienyllithium (1M soln in THF) and stirring at 40° C. for 2 hours. Crude material, following aqueous workup, was purified on a Biotage NH₂ column employing hexanes with a CH₂Cl₂ gradient to yield 280 mg yellow crystalline solid. MS (M+H)=238, ¹H NMR (300 MHz, DMSO-D6) δ ppm 5.94 (s, 1H) 7.02-7.12 (m, 1H) 7.16-7.25 (m, 1H) 7.36-7.45 (m, 1H) 7.58 (d, J=3.01 Hz, 1H) 7.72-7.87 (m, 2H) 8.04 (s, 0.4H) 8.11 (d, J=3.01 Hz, 0.6H) 10.45 (s, 0.6H) 10.93 (s, 0.4H).

Step 3. Preparation of 7-chloro-5-(4-fluorophenyl)-3-thien-2-yl-1,3-dihydro-2H-pyrido[2,3-e][1,4]diazepin-2-one: Prepared as for Example 30 Step 2 employing 5-chloro-3-[imino(thien-2-yl)methyl]pyridin-2-amine (280 mg, 1.18 mmol) and methyl amino(thien-2-yl)acetate HCl salt (243 mg, 1.18 mmol). Pure product was precipitated from MeOH following aqueous workup and collected by filtration, 63 mg, MS (M+H)=360, ¹H NMR (300 MHz, DMSO-D6) δ ppm 5.32 (s, 1H) 7.05 (d, J=5.28 Hz, 2H) 7.13-7.21 (m, 1H) 7.23 (d, J=3.77 Hz, 1H) 7.54 (d, J=4.52 Hz, 1H) 7.83 (d, J=5.28 Hz, 1H) 8.28 (s, 1H) 8.76 (d, J=3.01 Hz, 1H) 11.35 (s, 1H).

Step 4. Preparation of 7-chloro-N-methyl-3,5-dithien-2-yl-3H-pyrido[2,3-e][1,4]diazepin-2-amine: Prepared as in Example 30 Step 3 employing 7-chloro-5-(4-fluorophenyl)-3-thien-2-yl-1,3-dihydro-2H-pyrido[2,3-e][1,4]diazepin-2-one (100 mg), 3 eq TiCl₄, and 5 mL N-methylamine (2M soln in THF) to yield the title compound, MS (M+H)=359, ¹H NMR (300 MHz, DMSO-D6) δ ppm 2.84 (d, J=4.90 Hz, 3H) 5.28 (s, 1H) 7.17-7.32 (m, 4H) 7.68 (d, J=4.90 Hz, 1H) 7.86 (d, J=4.90 Hz, 1H) 8.25 (d, J=2.64 Hz, 1H) 8.79 (d, J=2.26 Hz, 1H).

### Example 37

### N-cyclopropyl-5-(2-furyl)-3-thien-2-yl-3H-pyrido[2,3-e][1,4]diazepin-2-amine

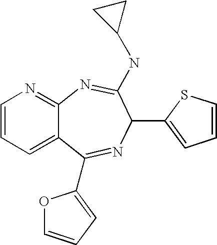

Prepared as in Example 30 Step 3 employing 5-(2-furyl)-3-thien-2-yl-1,3-dihydro-2H-pyrido[2,3-e][1,4]diazepin-2-one (40 mg), 3 eq TiCl₄, 64 μl cyclopropylamine, and 5 mL anhydrous THF to yield the title compound, MS (M+H)=349, ¹H NMR (300 MHz, DMSO-D6) δ ppm 0.27-0.41 (m, 1H) 0.50-0.66 (m, 3H) 2.79 (m, 1H) 4.76 (s, 1H) 6.10 (d, J=4.14 Hz, 1H) 6.65-6.74 (m, 1H) 6.84 (m, 1H) 7.06-7.16 (m, 2H) 7.21 (d, J=3.39 Hz, 1H) 7.60 (d, J=5.28 Hz, 1H) 7.93-8.01 (m, 2H) 8.62 (m, 1H).

### Example 38

### N-ethyl-5-(2-furyl)-3-thien-2-yl-3H-pyrido[2,3-e][1,4]diazepin-2-amine

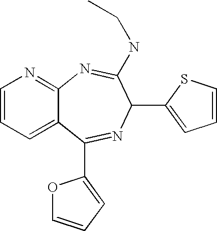

Prepared as in Example 30 Step 3 employing 5-(2-furyl)-3-thien-2-yl-1,3-dihydro-2H-pyrido[2,3-e][1,4]diazepin-2-one (60 mg), 3 eq TiCl₄, and 5 mL ethylamine (2M solution in THF) to yield the title compound, MS (M+H)=337, ¹H NMR (300 MHz, DMSO-D6) δ ppm 1.00 (t, 3H) 3.23 (m, 2H) 4.79 (s, 1H) 6.18-6.63 (m, 1H) 6.65-6.68 (m, 1H) 6.84 (d, 1H) 7.06 (m, 1H) 7.15-7.18 (m, 1H) 7.21-7.23 (m, 1H) 7.62 (d, 1H) 7.94-7.98 (m, 2H) 8.60 (m, 1H).

### Example 39

### 5-(2-furyl)-N-prop-2-ynyl-3-thien-2-yl-3H-pyrido[2,3-e][1,4]diazepin-2-amine

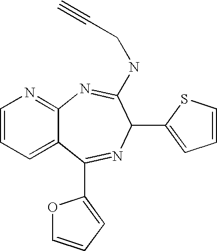

Prepared as in Example 30 Step 3 employing 5-(2-furyl)-3-thien-2-yl-1,3-dihydro-2H-pyrido[2,3-e][1,4]diazepin-2-one (60 mg), 3 eq TiCl₄, 107 μL propargylamine, and 5 mL anhydrous THF to yield the title compound, MS (M+H)=347, ¹H NMR (300 MHz, DMSO-D6) δ ppm 2.54 (s, 2H) 3.14 (s, 1H) 5.34 (s, 1H) 6.75 (d, J=1.88 Hz, 1H) 6.98 (d, J=3.39 Hz, 1H) 7.19-7.31 (m, 2H) 7.46 (dd, J=7.54, 6.03 Hz, 1H) 7.70 (d, J=4.90 Hz, 1H) 8.03 (s, 2H) 8.61 (d, J=7.91 Hz, 1H) 8.73 (d, J=4.90 Hz, 1H).

### Example 40

### 2-azetidin-1-yl-5-(2-furyl)-3-thien-2-yl-3H-pyrido[2,3-e][1,4]diazepine

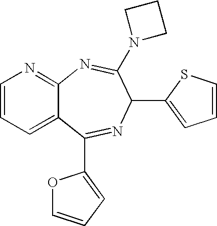

Prepared as in Example 30 Step 3 employing 5-(2-furyl)-3-thien-2-yl-1,3-dihydro-2H-pyrido[2,3-e][1,4]diazepin-2-one (60 mg), 3 eq TiCl₄, 104 μL azetidine, and 5 mL anhydrous THF to yield the title compound, MS (M+H)=349, ¹H NMR (300 MHz, DMSO-D6) δ ppm 1.99 (s, 1H) 2.08 (s, 1H) 3.15-3.30 (1,1H) 3.62 (s, 1H) 3.85 (s, 1H) 3.96-4.11 (m, 1H) 4.70 (s, 1H) 6.70 (dd, J=3.39, 1.51 Hz, 1H) 6.81-6.87 (m, 1H) 7.03-7.17 (m, 2H) 7.29 (d, J=3.77 Hz, 1H) 7.58 (d, J=4.90 Hz, 1H 7.97 (s, 1H) 7.99 (dd, J=7.91, 1.88 Hz, 1H) 8.61 (dd, J=4.52, 1.88 Hz, 1H).

### Example 41

### 7-fluoro-5-(2-furyl)-N-methyl-3-thien-2-yl-3H-pyrido[2,3-e][1,4]diazepin-2-amine

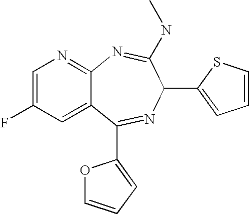

Step 1. Preparation of 2-amino-5-fluoronicotinonitrile: In a microwave vessel was combined 3-bromo-5-fluoropyridin-2-amine (2.0 g, 0.0105 mmol), Zn(CN)₂ (737 mg), DPPF (25 mg), and 5 mL wet DMF. The solution was then purged with N₂(g) prior to the addition of Pd₂ dba (20 mg). The reaction was heated at 120° C. for 40 mins in a microwave reactor. Crude material was purified by flash silica column to yield 570 mg solid yellow product, MS (M+H)=138, ¹H NMR (300 MHz, DMSO-D6) δ ppm 6.86 (s, 2H) 7.98 (dd, J=8.29, 3.01 Hz, 1H) 8.26 (d, J=3.01 Hz, 1H).

Step 2. Preparation of 5-fluoro-3-[2-furyl(imino)methyl]pyridin-2-amine: To a solution of furan (16.6 mmol) in 50 mL THF at −78° C. was slowly added 17 mmol n-BuLi (6.8 mL of a 2.5 M solution in hexanes). The solution was transferred to an ice water bath, and allowed to stir for 1.5 hours. The furanyllitium solution was returned to the −78° C. bath prior to the addition of 2-amino-5-fluoronicotinonitrile (570 mg, 4.16 mmol) dissolved in 15 mL THF. The combined mixture was then returned to the 0° C. ice water bath. After 30 minutes at 0° C. the reaction was quenched with NH₄Cl (aq) and the organic layer washed several times with water, dried (MgSO₄), and concentrated under vacuum to yield 800 mg product, MS (M+H)=206, ¹H NMR (300 MHz, DMSO-D6) δ ppm 6.69 (dd, J=3.39, 1.88 Hz, 1H) 6.94 (d, J=3.39 Hz, 1H) 7.30 (s, 2H) 7.67 (dd, J=9.80, 3.01 Hz, 1H) 7.96 (d, J=1.51 Hz, 1H) 8.12 (d, J=3.01 Hz, 1H) 10.85 (s, 1H).

Step 3. Preparation of 7-fluoro-5-(2-furyl)-3-thien-2-yl-1,3-dihydro-2H-pyrido[2,3-e][1,4]diazepin-2-one: Prepared as for Example 30 Step 2 employing 5-fluoro-3-[2-furyl(imino)methyl]pyridin-2-amine (800 mg, 3.9 mmol) and methyl amino(thien-2-yl)acetate HCl salt (800 mg, 1.18 mmol). Aqueous workup, followed by flash silica purification and MeOH precipitation yielded 40 mg gray product, MS (M+H)=328, ¹H NMR (300 MHz, DMSO-D6) δ ppm 5.28 (s, 1H) 6.70 (s, 1H) 6.94 (d, J=3.39 Hz, 1H) 7.00-7.15 (m, 2H) 7.54 (d, J=4.14 Hz, 1H) 7.97 (s, 1H) 8.15 (dd, J=8.48, 2.83 Hz, 1H) 8.74 (d, J=3.01 Hz, 1H) 11.26 (s, 1H).

Step 4. Preparation of 7-fluoro-5-(2-furyl)-N-methyl-3-thien-2-yl-3H-pyrido[2,3-e][1,4]diazepin-2-amine: Prepared as in Example 30 Step 3 employing 7-fluoro-5-(2-furyl)-3-thien-2-yl-1,3-dihydro-2H-pyrido[2,3-e][1,4]diazepin-2-one (35 mg), 3 eq TiCl₄, and 5 mL N-methylamine (2M soln in THF) to yield the title compound, MS (M+H)=341, ¹H NMR (300 MHz, DMSO-D6) δ ppm 2.72 (d, J=4.52 Hz, 3H) 4.93 (s, 1H) 6.51-6.61 (m, 1H) 6.69 (dd, J=3.39, 1.88 Hz, 1H) 6.94 (d, J=3.39 Hz, 1H) 7.17 (dd, J=5.09, 3.58 Hz, 1H) 7.23 (d, J=3.39 Hz, 1H) 7.63 (d, J=4.14 Hz, 1H) 7.93 (dd, J=8.67, 3.01 Hz, 1H) 7.96 (s, 1H) 8.65 (d, J=3.01 Hz, 1H).

### Example 42

### N-methyl-3-pyridin-3-yl-5-thien-2-yl-3H-pyrido[2,3-e][1,4]diazepin-2-amine

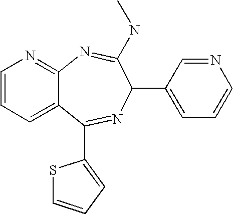

Step 1. Preparation of 5-(2-thienyl)-3-pyridin-3-yl-1,3-dihydro-2H-pyrido[2,3-e][1,4]diazepin-2-one: Prepared as for Example 30 Step 2 employing 3-[2-thienyl(imino)methyl]pyridin-2-amine (400 mg, 1.97 mmol [see Example 9 Step 1]) and methyl amino(pyridin-3-yl)acetate mono HCl salt (465 mg, 1.97 mmol [U.S. Pat. No. 6,191,166 B1]). Aqueous workup, followed by flash silica purification and MeOH precipitation yielded 120 mg solid product, MS (M+H)=353, ¹H NMR (300 MHz, DMSO-D6) δ ppm 5.04 (s, 1H) 7.17 (s, 2H) 7.47 (s, 2H) 7.82 (s, 1H) 7.99 (s, 1H) 8.24 (s, 1H) 8.56 (s, 1H) 8.72 (s, 2H) 11.27 (s, 1H).

Step 2. Preparation of N-methyl-3-pyridin-3-yl-5-thien-2-yl-3H-pyrido[2,3-e][1,4]diazepin-2-amine: Prepared as in Example 30 Step 3 employing 5-(2-thienyl)-3-pyridin-3-yl-1,3-dihydro-2H-pyrido[2,3-e][1,4]diazepin-2-one (60 mg), 3 eq TiCl₄, and 5 mL N-methylamine (2M soln in THF) to yield the title compound, MS (M+H)=334, ¹H NMR (300 MHz, DMSO-D6) δ ppm 2.82 (d, J=4.52 Hz, 3H) 5.29 (s, 1H) 7.20-7.27 (m, 1H) 7.30 (d, J=3.77 Hz, 1H) 7.43-7.54 (m, 1H) 7.75 (dd, J=8.29, 5.28 Hz, 1H) 7.89 (t, J=4.90 Hz, 2H) 8.28 (d, J=8.29 Hz, 1H) 8.64 (d, J=7.54 Hz, 1H) 8.77 (dd, J=8.67, 5.65 Hz, 2H) 8.98 (s, 1H).

### Example 43

### 5-(2-furyl)-N,8-dimethyl-3-thien-2-yl-3H-pyrido[2,3-e][1,4]diazepin-2-amine

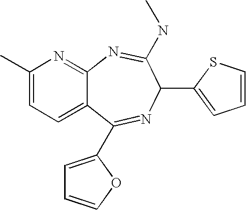

Step 1. Preparation of 3-[2-furyl(imino)methyl]-6-methylpyridin-2-amine: Prepared as in Example 41 Step 2 employing 2-amino-6-methylnicotinonitrile (950 mg, 7.14 mmol) and 4 eq furanyllithium, MS (M+H)=202, ¹H NMR (300 MHz, DMSO-D6) δ ppm 2.31 (s, 3H) 6.49 (m, 1H) 6.67 (s, 1H) 6.81 (m, 1H) 7.53 (s, 2H) 7.68 (m, 1H) 7.93 (s, 1H) 10.49 (s, 1H).

Step 2. Preparation of 5-(2-furyl)-8-methyl-3-thien-2-yl-1,3-dihydro-2H-pyrido[2,3-e][1,4]diazepin-2-one: Prepared as for Example 30 Step 2 employing 3-[2-furyl(imino)methyl]-6-methylpyridin-2-amine (500 mg, 2.49 mmol) and methyl amino(thien-2-yl)acetate HCl salt (465 mg, 1.97 mmol). Product was isolated following aqueous workup and precipitation from MeOH to yield 159 mg red solid, MS (M+H)=324, ¹H NMR (300 MHz, DMSO-D6) δ ppm 2.56 (s, obscured by solvent, 3H) 5.16 (s, 1H) 6.68 (s, 1H) 6.81 (s, 1H) 7.06 (m, 2H) 7.22 (m, 1H) 7.53 (s, 1H) 7.95 (s, 2H) 11.10 (s, 1H).

Step 2. Preparation of 5-(2-furyl)-N,8-dimethyl-3-thien-2-yl-3H-pyrido[2,3-e][1,4]diazepin-2-amine: Prepared as in Example 30 Step 3 employing 5-(2-furyl)-8-methyl-3-thien-2-yl-1,3-dihydro-2H-pyrido[2,3-e][1,4]diazepin-2-one (60 mg), 3 eq TiCl₄, and 5 mL N-methylamine (2M soln in THF) to yield the title compound, MS (M+H)=337, ¹H NMR (300 MHz, DMSO-D6) δ ppm 2.70 (s, 3H) 2.85 (d, J=4.52 Hz, 3H) 5.33 (s, 1H) 6.74 (s, 1H) 6.95 (d, J=3.01 Hz, 1H) 7.18-7.27 (m, 2H) 7.33 (d, J=7.54 Hz, 1H) 7.69 (d, J=5.28 Hz, 1H) 7.86 (d, J=4.52 Hz, 1H) 8.02 (s, 1H) 8.49 (d, J=8.29 Hz, 1H).

### Example 44

### N,8-dimethyl-3,5-dithien-2-yl-3H-pyrido[2,3-e][1,4]diazepin-2-amine

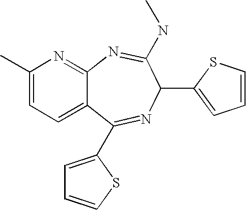

Step 1. Preparation of 3-[2-thienyl(imino)methyl]-6-methylpyridin-2-amine: Prepared as in Example 41 Step 2 employing 2-amino-6-methylnicotinonitrile (950 mg, 7.14 mmol) and 4 eq thienyllithium (1M solution in THF). The reaction was allowed to stir at room temperature for 3 hours prior to quenching with NH₄Cl (aq). After aqueous workup, the crude product was purified on a Biotage NH flash column to yield 640 mg solid yellow product, ˜90% pure by UV, MS (M+H)=218.

Step 2. Preparation of 5-(2-thienyl)-8-methyl-3-thien-2-yl-1,3-dihydro-2H-pyrido[2,3-e][1,4]diazepin-2-one: Prepared as for Example 30 Step 2 employing 3-[2-thienyl(imino)methyl]-6-methylpyridin-2-amine (650 mg, 3.0 mmol) and methyl amino(thien-2-yl)acetate HCl salt HCl salt (617 mg, 3.0 mmol). Product was isolated following aqueous workup and precipitated from MeOH to yield 302 mg pink solid, MS (M+H)=324, ¹H NMR (300 MHz, DMSO-D6) δ ppm 2.57 (s, 3H) 5.17 (s, 1H) 7.04 (m, 1H) 7.07 (s, 1H) 7.15 (m, 2H) 7.24 (d, J=7.54 Hz, 1H) 7.54 (s, 1H) 7.82 (s, 1H) 8.08 (d, J=7.54 Hz, 1H) 11.08 (s, 1H).

Step 3. Preparation of N,8-dimethyl-3,5-dithien-2-yl-3H-pyrido[2,3-e][1,4]diazepin-2-amine: Prepared as in Example 30 Step 3 employing 5-(2-thienyl)-8-methyl-3-thien-2-yl-1,3-dihydro-2H-pyrido[2,3-e][1,4]diazepin-2-one (90 mg), 3 eq TiCl₄, and 5 mL N-methylamine (2M soln in THF) to yield the title compound, MS (M+H)=353, ¹H NMR (300 MHz, DMSO-D6) δ ppm 2.51 (obscured by solvent, 3H) 2.70 (d, J=4.52 Hz, 3H) 4.79 (s, 1H) 6.30 (d, J=4.52 Hz, 1H) 6.95 (d, J=7.54 Hz, 1H) 7.17 (t, J=5.28 Hz, 4H) 7.62 (d, J=4.52 Hz, 1H) 7.76 (d, J=3.77 Hz, 1H) 7.92 (d, J=8.29 Hz, 1H).

### Example 45

### 2-(2,2-dimethylhydrazino)-3,5-dithien-2-yl-3H-pyrido[2,3-e][1,4]diazepine

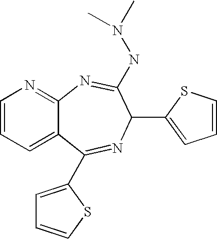

Preparation of 2-(2,2-dimethylhydrazino)-3,5-dithien-2-yl-3H-pyrido[2,3-e][1,4]diazepine: Prepared as in Example 30 Step 3 employing 3,5-dithien-2-yl-1,3-dihydro-2H-pyrido[2,3-e][1,4]diazepin-2-one (80 mg), 3 eq TiCl₄, 151 μL dimethylhydrazine (188 mg, 1.97 mmol) and 5 mL THF to yield the title compound, MS (M+H)=368, ¹H NMR (300 MHz, DMSO-D6) δ ppm 2.32 (bs, 6H) 5.54 (bs, 1H) 6.98 (bs, 1H) 7.11-7.25 (m, 3H) 7.46 (s, 1H) 7.80 (m, 1H) 8.05 (m, 2H) 8.54 (bs, 1H) 8.90 (s, 1H).

### Example 46

### 5-(2-furyl)-N-methyl-3-(1,3-thiazol-5-yl)-3H-pyrido[2,3-e][1,4]diazepin-2-amine

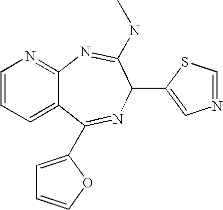

Step 1. Preparation of ethyl oxo(1,3-thiazol-5-yl)acetate: To a solution of 2-(trimethylsilyl)-1,3-thiazole (4.92 g, 31 mmol) in 50 mL Et₂O at −78° C. was added dropwise 1.1 eq n-BuLi (2.5 M solution in hexanes). The solution was stirred for 15 at −78° C. before the addition of diethyloxalate (5.49 g, 38 mmol) dissolved in 10 mL Et₂O. The solution was allowed to stir an additional 15 minutes prior to quenching cold with 50 mL 10% concentrated HCl. The reaction mixture was diluted with NaHCO₃ (aq) and product extracted with EtOAc. The combined organic fractions were washed with water, dried (MgSO₄), and concentrated. Product was isolated following flash silica purification to yield a yellow liquid, 2.0 g, ¹H NMR (300 MHz, DMSO-D6) δ ppm 1.27-1.39 (t, 3H) 4.29-4.44 (q, 2H) 8.85 (s, 1H) 9.53 (s, 1H).

Step 2. Preparation of ethyl 2-(hydroxyimino)(1,3-thiazol-5-yl)acetate: A solution of ethyl oxo(1,3-thiazol-5-yl)acetate (1.5 g, 8.1 mmol), sodium acetate (730 mg, 8.9 mmol), hydroxylamine hydrochloride salt (1 g, 14.6 mmol), and 50 mL of EtOH was refluxed for 1 hour. The solution was concentrated under vacuum, reconstituted in EtOAc, and washed with 5% concentrated HCl (aq) prior to drying (MgSO₄). Concentrating under vacuum yielded 700 mg bright yellow solid product, MS (M+H)=201, ¹H NMR (300 MHz, DMSO-D6) δ ppm 1.31 (t, J=6.78 Hz, 4H) 4.30-4.42 (m, 3H) 8.69 (s, 1H) 9.28 (s, 1H) 13.60 (s, 1H).

Step 3. Preparation of ethyl amino(1,3-thiazol-5-yl)acetate HCl salt: Aluminum foil (160 mg) was torn into small pieces and swirled in a beaker with 5 mL 10% H₂O/THF and 50 mg HgCl₂ for a few minutes. The foil (leaving behind the HgCl₂ solution) was then transferred to a solution of ethyl 2-(hydroxyimino)(1,3-thiazol-5-yl)acetate (400 mg, 2 mmol) in 30 mL 10% water/THF. The suspension was stirred at room temperature for 1.5 hours. The mixture was filtered through Celite, and the filtrate concentrated under vacuum to yield product as the free base (310 mg, 1.6 mmol). After characterization, the product was converted to the mono-HCl salt by addition of 1 eq HCl (from a 2M dioxane solution). MS (M+H)=187, ¹H NMR (300 MHz, DMSO-D6) δ ppm 1.12-1.22 (m, 3H) 4.14 (q, J=6.78 Hz, 3H) 4.88 (s, 1H) 7.82 (s, 1H) 9.00 (s, 1H).

Step 4. Preparation of 5-(2-furyl)-3-(1,3-thiazol-5-yl)-1,3-dihydro-2H-pyrido[2,3-e][1,4]diazepin-2-one: Prepared as for Example 30 Step 2 employing 3-[2-thienyl(imino)methyl]pyridin-2-amine (313 mg, 1.66 mmol) and ethyl amino(1,3-thiazol-5-yl)acetate HCl salt (1.66 mmol). Product was isolated following aqueous workup and precipitation from MeOH to yield 55 mg solid, MS (M+H)=311.

Step 5. Preparation of 5-(2-furyl)-N-methyl-3-(1,3-thiazol-5-yl)-3H-pyrido[2,3-e][1,4]diazepin-2-amine: Prepared as in Example 30 Step 3 employing 5-(2-furyl)-3-(1,3-thiazol-5-yl)-1,3-dihydro-2H-pyrido[2,3-e][1,4]diazepin-2-one (55 mg), 3 eq TiCl₄, and 5 mL N-methylamine (2M soln in THF) to yield the title compound, MS (M+H)=324, ¹H NMR (300 MHz, DMSO-D6) δ ppm 2.68 (d, J=4.52 Hz, 3H) 4.92 (s, 1H) 6.60-6.74 (m, 2H) 6.83 (d, J=3.77 Hz, 1H) 7.05 (dd, J=7.54, 4.52 Hz, 1H) 7.92-8.06 (m, 3H) 8.59 (d, J=4.52 Hz, 1H) 9.15 (s, 1H).

### Example 47

### N-methyl-3-(1,3-thiazol-5-yl)-5-thien-2-yl-3H-pyrido[2,3-e][1,4]diazepin-2-amine

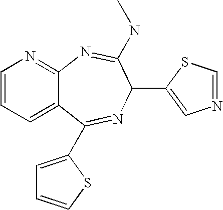

Step 1. Preparation of 3-(1,3-thiazol-5-yl)-5-thien-2-yl-1,3-dihydro-2H-pyrido[2,3-e][1,4]diazepin-2-one: Prepared as for Example 30 Step 2 employing 3-[2-thienyl(imino)methyl]pyridin-2-amine (849 mg, 4.27 mmol [see Example 9, Step 1]) and ethyl amino(1,3-thiazol-5-yl)acetate HCl salt (800 mg, 4.27 mmol). Product was precipitated from MeOH following aqueous workup to yield 60 mg brown solid, MS (M+H)=373.

Step 2. Preparation of N-methyl-3-(1,3-thiazol-5-yl)-5-thien-2-yl-3H-pyrido[2,3-e][1,4]diazepin-2-amine: Prepared as in Example 30 Step 3 employing 3-(1,3-thiazol-5-yl)-5-thien-2-yl-1,3-dihydro-2H-pyrido[2,3-e][1,4]diazepin-2-one (60 mg), 3 eq TiCl₄, and 5 mL N-methylamine (2M soln in THF) to yield the title compound, MS (M+H)=339, ¹H NMR (300 MHz, DMSO-D6) δ ppm 2.61 (d, J=4.52 Hz, 4H) 4.86 (s, 1H) 6.57 (d, J=4.52 Hz, 1H) 6.99 (dd, J=7.54, 4.52 Hz, 1H) 7.05-7.14 (m, 2H) 7.69 (d, J=5.28 Hz, 1H) 7.95 (m, 2H) 8.54 (s, 1H) 9.08 (s, 1H).

### Example 48

### N-cyclopropyl-5-(2-furyl)-3-(1,3-thiazol-5-yl)-3H-pyrido[2,3-e][1,4]diazepin-2-amine

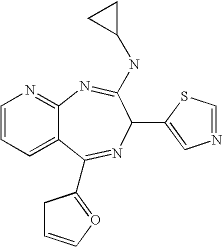

Preparation of N-cyclopropyl-5-(2-furyl)-3-(1,3-thiazol-5-yl)-3H-pyrido[2,3-e][1,4]diazepin-2-amine: Prepared as in Example 30 Step 3 employing 5-(2-furyl)-3-(1,3-thiazol-5-yl)-1,3-dihydro-2H-pyrido[2,3-e][1,4]diazepin-2-one (100 mg), 3 eq TiCl₄, 200 μL cyclopropylamine (165 mg), and 5 mL THF to yield the title compound, MS (M+H)=350, ¹H NMR (300 MHz, DMSO-D6) δ ppm 0.29-0.44 (m, 1H) 0.51-0.65 (m, 3H) 2.81 (d, J=3.77 Hz, 1H) 4.86 (s, 1H) 6.61 (d, J=3.77 Hz, 1H) 6.70 (s, 1H) 6.87 (d, J=3.01 Hz, 1H) 7.08 (dd, J=7.54, 4.52 Hz, 1H) 7.93-8.07 (m, 3H) 8.62 (d, J=4.52 Hz, 1H).

### Example 49

### N-methyl-3-(1H-pyrazol-4-yl)-5-thien-2-yl-3H-pyrido[2,3-e][1,4]diazepin-2-amine

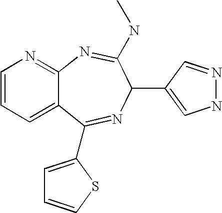

Step 1. Preparation of ethyl oxo(1H-pyrazol-4-yl)acetate: To a solution of 4-bromo-1H-pyrazole in 40 mL THF at −78° C. was added in portions 30 mL n-BuLi (1.6M solution in hexanes). The solution was allowed to warm to room temperature. After stirring at room temperature for 1.5 hours, the mixture was returned to −78° C. and diethyloxalate (2.7 mL, 10 mmol) dissolved in 2.5 mL THF was added. The solution was stirred for an additional 20 minutes before quenching cold with NH₄Cl(aq). Product was extracted from the aqueous layer with EtOAc and the combined fractions concentrated and dried (MgSO₄). Product was isolated by flash silica to yield 240 mg white solid, MS (M+H)=169, ¹H NMR (300 MHz, DMSO-D6) δ ppm 1.03-1.14 (m, 4H) 4.13 (q, J=7.54 Hz, 3H) 7.92 (s, 1H) 8.42 (s, 1H) 13.54 (bs, 1H).

Step 2. Preparation of ethyl 2-(hydroxyimino)(1H-pyrazol-4-yl)acetate: Prepared in the same manner as Example 46 Step 2 employing ethyl oxo(1H-pyrazol-4-yl)acetate (230 mg, 1.37 mmol) yielding 200 mg white solid following aqueous workup, MS (M+H)=184, ¹H NMR (300 MHz, DMSO-D6) δ ppm 1.30-1.42 (m, 3H) 4.35 (dq, J=10.93, 7.16 Hz, 2H) 7.86 (s, 1H) 8.30 (s, 1H) 11.42 (s, 1H) 12.52 (s, 1H).

Step 3. Preparation of ethyl amino(1H-pyrazol-4-yl)acetate mono HCl salt: Prepared in the same manner as Example 46 Step 3 employing ethyl 2-(hydroxyimino)(1H-pyrazol-4-yl)acetate (200 mg, 1 mmol), 100 mg aluminum foil, and 30 mg HgCl₂ to yield 150 mg solid white free base following Celite filtration, MS (M+H)=170, ¹H NMR (300 MHz, DMSO-D6) δ ppm 1.09 (t, J=7.16 Hz, 3H) 4.04-4.18 (m, 2H) 5.13 (bs, 1H) 7.63 (s, 2H) 8.62 (s, 2H). The mono HCl salt was obtained by the addition of 1 eq HCl (2M solution in dioxane) to the free base dissolved in a small amount of anhydrous EtOH.

Step 4. Preparation of 3-(1H-pyrazol-4-yl)-5-thien-2-yl-1,3-dihydro-2H-pyrido[2,3-e][1,4]diazepin-2-one: Prepared as for Example 30 Step 2 employing 3-[2-thienyl(imino)methyl]pyridin-2-amine (336 mg, 1.66 mmol [Example 9, Step 1) and ethyl amino(1H-pyrazol-4-yl)acetate mono HCl salt (338 mg, 1.66 mmol). Product was filtered out of a biphasic mixture of NH₄Cl (aq) and EtOAc and washed with MeOH to yield 165 mg solid brown product, MS (M+H)=310, ¹H NMR (300 MHz, DMSO-D6) δ ppm 4.83 (s, 1H) 7.08-7.21 (m, 3H) 7.35 (m, 1H) 7.78 (d, J=3.77 Hz, 2H) 8.19 (m, 1H) 8.67 (m, 1H) 11.09 (s, 1H) 12.74 (s, 1H).

Step 4. Preparation of N-methyl-3-(1H-pyrazol-4-yl)-5-thien-2-yl-3H-pyrido[2,3-e][1,4]diazepin-2-amine: Prepared as in Example 30 Step 3 employing 3-(1H-pyrazol-4-yl)-5-thien-2-yl-1,3-dihydro-2H-pyrido[2,3-e][1,4]diazepin-2-one (140 mg), 3 eq TiCl₄, and 5 mL N-methylamine (2M solution in THF) to yield the title compound, MS (M+H)=323, ¹H NMR (300 MHz, DMSO-D6) δ ppm 2.69 (d, J=4.52 Hz, 3H) 4.53 (s, 1H) 6.38 (d, J=4.52 Hz, 1H) 7.05 (dd, J=7.91, 4.90 Hz, 1H) 7.11-7.19 (m, 2H) 7.67 (s, 1H) 7.75 (m, 1H) 7.87 (s, 1H) 8.02 (m, 1H) 8.59 (m, 1H) 12.97 (s, 1H).

### Example 50

### N-methyl-3,5-di-2-thienyl-3H-pyrido[2,3-e][1,4]diazepin-2-amine

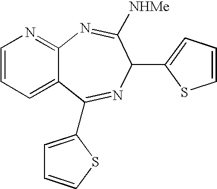

Steps 1, 2 and 3 were done according to literature procedure (Timur Gungor et al. Journal of Organometallic Chemistry, 215, (1981), 139-150) and were prepared as follows:

### Step 1: (2-Fluoropyridin-3-yl)(2-thienyl)methanol

To a solution of 2-fluoropyridine (2.0 g, 20.6 mmol) in anhydrous THF (5 mL) was added a solution of 2M LDA (heptane/THF, 12 mL, 24 mmol) at −78° C. under nitrogen. An orange suspension was formed. To above suspension was added a solution of 2-thiophene carboxaldehyde in THF (5 mL). The reaction was stirred at −78° C. for 2 hrs and was allowed to warm to room temperature overnight. The reaction was worked up as following: the reaction mixture was poured into an ice water and extracted with ethyl acetate (3×100 mL). The combined organic layer was dried over magnesium sulfate and purified on Flash Master using ethyl acetate/hexane as eluent. The product was dried under reduced pressure to yield a yellow oil (2.6 g, 63%). ES (M+H⁺)⁺210.

### Step 2: (2-Fluoropyridin-3-yl)(2-thienyl)methanone

(2-Fluoropyridin-3-yl)(2-thienyl)methanol (2.6 g, 12.3 mmol) was refluxed with manganese (IV) oxide (3.0 g, 33.2 mmol) in THF (14 mL) for 48 hrs. The reaction mixture was filtered and the product was purified on Flash Master using ethyl acetate/hexane as eluant. The desired fractions were collected, concentrated and was dried under reduced pressure to give (2-fluoropyridin-3-yl)(2-thienyl)methanone as a yellow solid (2.25 g, 87%). ES (M+H⁺)⁺208.

### Step 3: (2-Aminopyridin-3-yl)(2-thienyl)methanone

(2-Fluoropyridin-3-yl)(2-thienyl)methanone (2.25 g, 10.87 mmol) was treated with ammonia methanol solution (or ammonia hydroxide) with moderate heating (or conducted in a bomb reaction at 40° C.). After heating overnight, the reaction was concentrated and the reaction mixture was purified on Flash Master using ethyl acetate/hexane as eluant. The desired fractions were collected, concentrated and was dried under reduced pressure to give (2-aminopyridin-3-yl)(2-thienyl)methanone as a yellow solid (1.73 g, 78%). ES (M+H)⁺205

### Step 4: 3,5-Di-2-thienyl-1,3-dihydro-2H-pyrido[2,3-e][1,4]diazepin-2-one

To a solution of (2-aminopyridin-3-yl)(2-thienyl)methanone (0.88 g, 4.31 mmol) and N-Boc amino(2-thienyl)acetic acid (2.37 g, 9.22 mmol) in anhydrous dichloromethane (15 mL) was added a solution of DCC (2.97 g, 8 mmol) in anhydrous dichloromethane (10 mL) at 0° C. The reaction was stirred at 0° C. for 3 hrs and was allowed to warm to room temperature overnight. The suspension was filtered and the filtrate was concentrated and purified on Flash master using ethyl acetate/hexane as eluant yielding a white solid (0.95 g, 50%).

### Step 5

The coupling product was then treated with a TFA solution (30 mL, 30%) in dichloromethane at 0° C. for 1 hr and continued stirred for 1 hr at room temperature. After de-protection was completed (monitored by LC-MS) the reaction mixture was concentrated to remove all solvent and TFA.

### Step 6: 3,5-di-2-thienyl-1,3-dihydro-2H-pyrido[2,3-e][1,4]diazepin-2-one

The oil like de-protected product was then dissolved with 30 mL of acetic acid and mixed with ammonium acetate solid. The suspension was heated to 60° C. for 12 hrs. After removing of acetic acid, product 3,5-di-2-thienyl-1,3-dihydro-2H-pyrido[2,3-e][1,4]diazepin-2-one was purified on Flash master using ethyl acetate/hexane as eluant and dried under reduced pressure to give a brown solid (0.48 g, 69%) ¹H NMR (300 MHz, DMSO-d6) δ ppm 5.55 (d, 1H); 7.15-7.70 (m, 4H); 7.37 (m, 1H); 7.52 (d, 1H); 7.80 (d, 1H); 8.20 (d, 1H); 8.60 (d, 1H); 11.19 (s, 1H). ES (M+H⁺)⁺=326

### Step 7: 3,5-Di-2-thienyl-1,3-dihydro-2H-pyrido[2,3-e][1,4]diazepine-2-thione

3,5-Di-2-thienyl-1,3-dihydro-2H-pyrido[2,3-e][1,4]diazepin-2-one (0.48 g, 1.476 mmol) was refluxed with Lawesson's reagent (1.0 g, 2.47 mmol) in dioxane for 12 hrs. The reaction was then concentrated and the product was purified on Flash master using ethyl acetate/hexane as eluant. The desired fractions were dried under reduced pressure to give a yellow solid (0.19 g, 38%). ES (M+H)⁺=342

### Step 8: N-Methyl-3,5-di-2-thienyl-3H-pyrido[2,3-e][1,4]diazepin-2-amine

3,5-Di-2-thienyl-1,3-dihydro-2H-pyrido[2,3-e][1,4]diazepine-2-thione (0.19 g, 0.557 mmol) was dissolved in a 2M methylamine solution (10 mL, 20 mmol) in THF. The mixture was heated to 60° C. for 15 min. The reaction mixture was concentrated and purified on Flash Master using ethyl acetate/hexane as eluant and dried under reduced pressure to give a white solid (55 mg, 31%) ¹H NMR (300 MHz, CHLOROFORM-d): δ ppm 2.94 (d, J=4.58 Hz, 3H); 4.92 (s, 1H); 5.13 (s, br, 1H); 7.0-7.2 (m, 5H); 7.47 (dd, J=13.6, 4.54 Hz, 2H); 8.05 (d, J=7.59 Hz, 1H); 8.71 (d, J=3.70 Hz, 1H). ES (M+H⁺)⁺=339

### Example 51

### 5-cyclohexyl-N-methyl-3-thien-2-yl-3H-pyrido[2,3-e][1,4]diazepin-2-amine

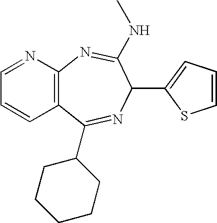

### Step 1: cyclohexyl(2-fluoropyridin-3-yl)methanol

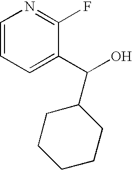

To a solution of 2-fluoropyridine (4.0 g, 41.2 mmol) in THF (40 mL) at −78 C was added 2M LDA (25 mL). After 1 h, cyclohexane carboxaldehyde (5.05 g, 45 mmol) in THF (20 mL) was added. After warming to slowly warm to RT over 4 h, the reaction was concentrated. The residue was dissolved in ethylacetate, washed with 1 N HCl, NaHCO₃ (sat'd), & brine. The organic solution was dried (Na₂SO₄) filtered and concentrated. This residue was purified by flash chromatography using a FlashMaster™ yielding 2.72 g, ES (M+H)⁺=209.

### Step 2: cyclohexyl(2-fluoropyridin-3-yl)methanone

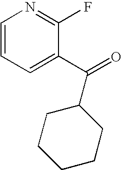

To a solution of cyclohexyl(2-fluoropyridin-3-yl)methanol (2.10 g, 10 mmol) in CH₂Cl₂ (40 mL) were added 4 A molecular sieves powder (1.0 g) and PCC (2.8 g). After 3 h, the reaction was diluted with CH₂Cl₂ and filtered through Celite. This residue was concentrated and purified by flash chromatography using a FlashMaster™ yielding 1.60 g, ES (M+H)⁺=207.

### Step 3: (2-aminopyridin-3-yl)(cyclohexyl)methanone

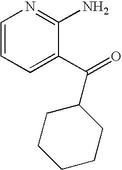

A solution of cyclohexyl(2-fluoropyridin-3-yl)methanone (3.56 g) in 7N NH₃/MeOH (30 mL) was heated overnight (Tb=100° C.) in a high pressure vessel. The reaction was cooled and concentrated. The residue was dissolved in ethylacetate, washed NaHCO₃ (sat'd), & brine. The organic solution was dried (Na₂SO₄) filtered and concentrated. This residue was purified by flash chromatography using a FlashMaster™ yielding 1.84 g, ES (M+H)⁺=204.

### Step 4: tert-butyl 2-{[3-(cyclohexylcarbonyl)pyridin-2-yl]amino}-2-oxo-1-thien-2-ylethylcarbamate

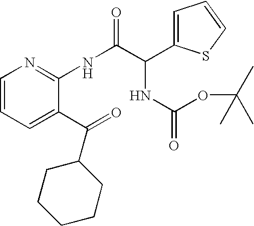

To a solution of (2-aminopyridin-3-yl)(cyclohexyl)methanone (1.84 g, 9.0 mmol) in THF (25 mL) were added DCC (2.55 g) and a 1M solution of [(tert-butoxycarbonyl)amino](thien-2-yl)acetic acid (9.0 mL) in CH₂Cl₂. After 5 h, the reaction additional DCC (2.60 g) and 1M solution of [(tert-butoxycarbonyl)amino](thien-2-yl)acetic acid (9.0 mL) were added. After stirring overnight at RT, the reaction was diluted with CH₂Cl₂ and filtered and concentrated ES (M+H)⁺=444.

### Step 5: 2-amino-N-[3-(cyclohexylcarbonyl)pyridin-2-yl]-2-thien-2-ylacetamide

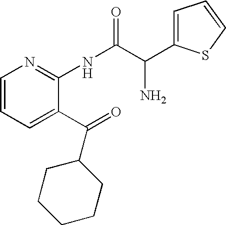

The crude residue from step 5 was dissolved in 30% TFA/CH₂Cl₂. After 1 h, the reaction was concentrated. ES (M+H)⁺=344.

### Step 6: 5-cyclohexyl-3-thien-2-yl-1,3-dihydro-2H-pyrido[2,3-e][1,4]diazepin-2-one

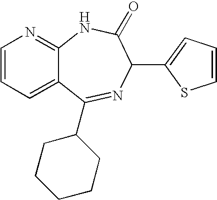

The residue from step 5 was redissolved in acetic acid (100 mL) and the pH adjusted with NH₄OAc (solid) until pH=5. After stirring overnight at RT, the reaction was diluted with ethylacetate and washed with 4N NaOH, NaHCO₃ (sat'd), & brine. The organic solution was dried (Na₂SO₄) filtered and concentrated. This residue was suspended in methanol and filtered yielding 0.57 g, ES (M+H)⁺=326.

### Step 7

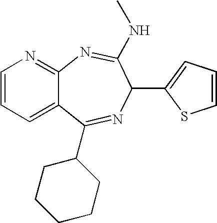

To a suspension of 5-cyclohexyl-3-thien-2-yl-1,3-dihydro-2H-pyrido[2,3-e][1,4]diazepin-2-one (195 mg) in 2M methylamine/THF (4 mL) was added 1M TiCl₄ in CH₂Cl₂. The reaction was heated in a microwave reactor for 30 min at 80 C. The reaction was diluted with ethylacetate washed with NaHCO₃ (sat'd), & brine. The organic solution was dried Na₂SO₄) filtered and concentrated. This residue was purified by flash chromatography using a FlashMaster™ yielding 0.15 g, ES (M+H)⁺=339. ¹H NMR (400 MHz, CHLOROFORM-D) δ ppm 1.14 (d, J=12.23 Hz, 1H) 1.22-1.33 (m, 2H) 1.38 (d, J=12.72 Hz, 1H) 1.55-1.64 (m, 2H) 1.69 (d, J=9.78 Hz, 2H) 1.78 (dd, J=10.39, 2.81 Hz, 1H) 1.91 (d, J=12.96 Hz, 1H) 2.05 (d, J=13.45 Hz, 1H) 2.83 (m, 1H) 2.92 (d, 3H) 4.72 (s, 1H) 4.97 (d, J=4.65 Hz, 1H) 6.98 (dd, J=7.83, 4.40 Hz, 1H) 7.07-7.15 (m, 2H) 7.39 (d, J=4.89 Hz, 1H) 7.91 (d, J=7.83 Hz, 1H) 8.60 (d, J=4.65 Hz, 1H); ES (MH⁺)⁺=339.

### Example 52

### 2-azetidin-1-yl-5-cyclohexyl-3-thien-2-yl-3H-pyrido[2,3-e][1,4]diazepine

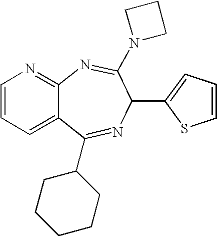

Using an analogous procedure to that described in Example 51, the titled compound was made from 5-cyclohexyl-3-thien-2-yl-1,3-dihydro-2H-pyrido[2,3-e][1,4]diazepin-2-one except that azetidine was used in step 7. ¹H NMR (400 MHz, CHLOROFORM-D) δ ppm 1.15 (d, J=11.98 Hz, 1H) 1.24-1.33 (m, 3H) 1.37 (dd, J=12.47, 2.93 Hz, 1H) 1.58 (d, J=12.96 Hz, 2H) 1.70 (d, J=9.05 Hz, 3H) 1.78-1.89 (m, 1H) 1.90-1.96 (m, 1H) 2.01-2.13 (m, 3H) 2.83-2.92 (m, 1H) 3.35 (s, 1H) 3.72 (s, 1H) 4.01 (s, 1H) 4.26 (s, 1H) 6.94 (dd, J=7.70, 4.77 Hz, 1H) 7.03-7.08 (m, 1H) 7.11 (s, 1H) 7.31 (d, J=5.14 Hz, 1H) 7.90 (dd, J=7.70, 1.59 Hz, 1H) 8.58 (d, J=2.93 Hz, 1H); ES (MH⁺)⁺=365.

### Examples 53

### 5-cyclopentyl-N-methyl-3-thien-2-yl-3H-pyrido[2,3-e][1,4]diazepin-2-amine

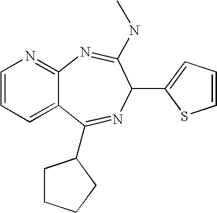

Using an analogous procedure to that described in Example 51, the titled compound was formed from cyclopentanecarbaldehyde. ¹H NMR (400 MHz, CHLOROFORM-D) δ ppm 1.44 (m, J=7.46, 4.52 Hz, 1H) 1.57-1.81 (m, 5H) 1.97 (dd, J=12.59, 5.26 Hz, 1H) 2.24 (dd, J=12.96, 7.83 Hz, 1H) 2.88-2.96 (m, 3H) 3.35-3.44 (m, 1H) 4.75 (s, 1H) 4.95 (d, J=3.91 Hz, 1H) 6.98 (dd, J=7.70, 4.77 Hz, 1H) 7.07-7.14 (m, 3H) 7.21-7.29 (m, 2H) 7.39 (d, J=4.89 Hz, 1H) 7.90-7.97 (m, 1H) 8.58-8.65 (m, 1H); ES (MH⁺)⁺=325; mp=183-187.

### Example 54

### N-methyl-5-phenyl-3-(2-thienyl)-3H-1,4-benzodiazepin-2-amine

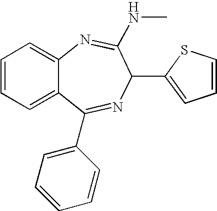

Using an analogous procedure to that described in Example 50 using steps 4 to 8, the titled compound was formed from (2-aminophenyl)(phenyl)methanone. ¹H NMR (300, DMSO-d₆) δ 2.70 (s, 3H), 4.71 (s, 1H), 6.07 (s, 1H), 6.99-7.58 (cp, 12H); MS (ES+) 332/333.

### Example 55

### N-methyl-3,5-diphenyl-3H-1,4-benzodiazepin-2-amine

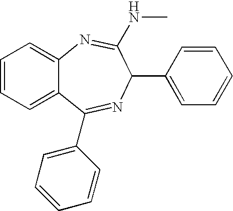

Using an analogous procedure to that described in Example 50 using steps 4 to 8, the titled compound was formed from (2-aminophenyl)(phenyl)methanone. ¹H NMR (300, CDCl₃) δ 2.83 (d, 3H, J=4.9), 4.63 (s, 1H), 6.99 (m, 1H), 7.38-7.67 (m, 9H), 7.71 (d, 2H, J=1.4), 7.83 (d, 2H, J=7.3); MS (ES+) 326/327.

### Example 56

### 7-chloro-N-methyl-3,5-diphenyl-3H-1,4-benzodiazepin-2-amine

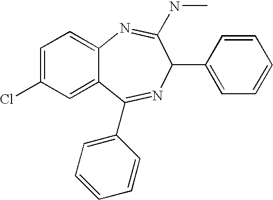

Using an analogous procedure to that described in Example 50 using steps 4 to 8, the titled compound was formed from (2-amino-5-chlorophenyl)(phenyl)methanone. ¹H NMR (300, CDCl₃) δ 2.83 (d, 3H, J=4.9), 4.51 (d, 1H, J=4.9), 4.61 (s, 1H), 7.31-7.67 (m, 9H), 7.68 (d, 2H, J=6.4), 7.81 (d, 2H, J=7.2); MS (ES+) 360/362.

### Example 57

### N-methyl-5-phenyl-3-thien-3-yl-3H-1,4-benzodiazepin-2-amine

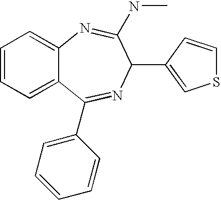

Using an analogous procedure to that described in Example 50 using steps 4 to 8, the titled compound was formed from (2-aminophenyl)(phenyl)methanone. ¹H NMR (300, CDCl₃) δ 2.85 (s, 3H), 4.70 (s, 1H), 5.39 (s, 1H), 6.99 (m, 1H), 7.25-7.48 (m, 9H), 7.67 (d, J=6.22, 2H), 7.89 (s, 1H); MS (ES+) 332/333.

### Example 58

### N-ethyl-5-phenyl-3-thien-2-yl-3H-1,4-benzodiazepin-2-amine

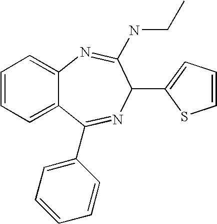

Using an analogous procedure to that described in Example 50 using steps 4 to 8, the titled compound was formed from (2-aminophenyl)(phenyl)methanone except that ethylamine was used in step 8. ¹H NMR (300, CDCl₃) δ 1.10 (t, 3H, J=7.25), 3.29 (m, 1H), 3.44 (m, 1H), 4.62 (s, 1), 4.85 (s, 1H), 6.98 (m, 1H), 7.20 (m, 2H), 7.44 (m, 7H), 7.69 (m, 2H); MS (ES+) 346/347.

### Example 59

### 5-phenyl-N-propyl-3-thien-2-yl-3H-1,4-benzodiazepin-2-amine

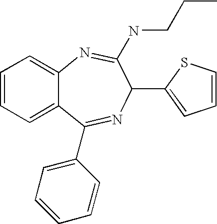

Using an analogous procedure to that described in Example 50 using steps 4 to 8, the titled compound was formed from (2-aminophenyl)(phenyl)methanone except that propylamine was used in step 8. ¹H NMR (300, CDCl₃) δ 0.87 (t, J=7.44, 3H), 1.51 (m, 2H), 3.18 (m, 1H), 3.39 (m, 1H), 4.87 (br s, 2H), 6.99 (m, 1H), 7.19 (m, 3H), 7.44 (m, 6H), 7.70 (m, 2H); MS (ES+) 360.

### Example 60

### N-isopropyl-5-phenyl-3-thien-2-yl-3H-1,4-benzodiazepin-2-amine

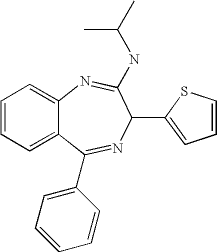

Using an analogous procedure to that described in Example 50 using steps 4 to 8, the titled compound was formed from (2-aminophenyl)(phenyl)methanone except that isopropylamine was used in step 8. ¹H NMR (300, CDCl₃) δ 1.01 (m, 3H), 1.21 (m, 3H), 4.11 (m, 1H), 4.64 (m, 1H), 4.82 (s, 1H), 6.97 (m, 1H), 7.17 (m, 3H), 7.30-7.44 (m, 6H), 7.69 (m, 2H); MS (ES+) 360.

### Example 61

### N-cyclopropyl-5-phenyl-3-thien-2-yl-3H-1,4-benzodiazepin-2-amine

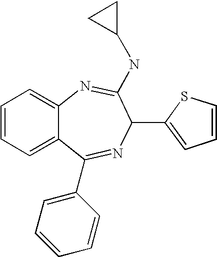

Using an analogous procedure to that described in Example 50 using steps 4 to 8, the titled compound was formed from (2-aminophenyl)(phenyl)methanone except that cyclopropylamine was used in step 8. ¹H NMR (300, CDCl₃) δ 0.37 (m, 1H), 0.49 (m, 1H), 0.76 (m, 2H), 2.78 (m, 1H), 4.81 (s, 1H), 4.95 (s, 1H), 7.00 (m, 1H), 7.17 (m, 2H), 7.44 (m, 7H), 7.69 (m, 2H); MS (ES+) 358/359.

### Example 62

### N,8-dimethyl-5-phenyl-3-thien-2-yl-3H-1,4-benzodiazepin-2-amine

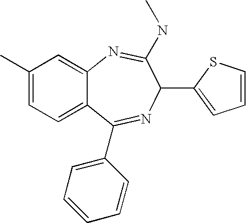

Using an analogous procedure to that described in Example 50 using steps 4 to 8, the titled compound was formed from (2-amino-4-methylphenyl)(phenyl)methanone. ¹H NMR (300, CDCl₃) δ 1.58 (s, 3H), 2.41 (s, 3H), 4.86 (s, 1H), 4.88 (m, 1H), 6.99 (m, 1H), 7.21 (m, 4H), 7.39 (m, 2H), 7.43 (m, 2H), 7.59 (d, 2H, J=8.10); MS (ES+) 346/347.

### Example 63

### N-allyl-5-phenyl-3-thien-2-yl-3H-1,4-benzodiazepin-2-amine

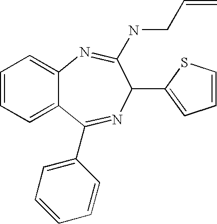

Using an analogous procedure to that described in Example 50 using steps 4 to 8, the titled compound was formed from (2-aminophenyl)(phenyl)methanone except that allylamine was used in step 8. ¹H NMR (300, DMSO-d₆) δ 3.83 (m, 2H), 4.74 (s, 1H), 4.99 (m, 2H), 5.79 (m, 1H), 6.01 (m, 1H), 6.99 (m, 1H), 7.23 (m, 3H), 7.49 (m, 7H), 7.63 (d, J=5.09, 1H); MS (ES+) 358/359.

### Example 64

### 5-phenyl-N-prop-2-ynyl-3-thien-2-yl-3H-1,4-benzodiazepin-2-amine

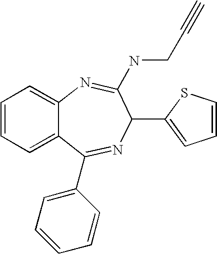

Using an analogous procedure to that described in Example 50 using steps 4 to 8, the titled compound was formed from (2-aminophenyl)(phenyl)methanone except that propargylamine was used in step 8. ¹H NMR (300, DMSO-d₆) δ 2.19 (s, 1H), 3.98 (m, 1H), 4.30 (m, 1H), 4.86 (s, 1H), 4.97 (br s, 1H), 7.01 (m, 1H), 7.21 (m, 2H), 7.45 (m, 7H), 7.69 (m, 2H); MS (ES+) 356.

### Example 65

### 5-phenyl-3-thien-2-yl-N-(2,2,2-trifluoroethyl)-3H-1,4-benzodiazepin-2-amine

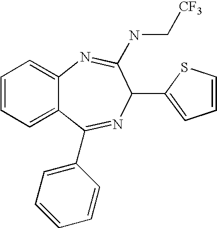

Using an analogous procedure to that described in Example 50 using steps 4 to 8, the titled compound was formed from (2-aminophenyl)(phenyl)methanone except that 2,2,2-trifluoroethylamine was used in step 8. ¹H NMR (300, CDCl₃) δ 3.95 (m, 1H), 4.46 (m, 1H), 4.95 (s, 1H), 5.25 (br s, 1H), 7.14 (m, 1H), 7.21 (m, 1H), 7.27 (m, 1H), 7.44 (m, 7H), 7.68 (d, 2H, J=6.97); MS (ES+) 400/401.

### Example 66

### N,N-dimethyl-5-phenyl-3-thien-2-yl-3H-1,4-benzodiazepin-2-amine

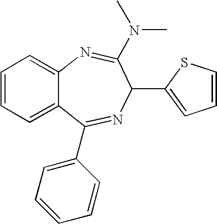

Using an analogous procedure to that described in Example 50 using steps 4 to 8, the titled compound was formed from (2-aminophenyl)(phenyl)methanone except that dimethylamine was used in step 8. ¹H NMR (300, DMSO-d₆) δ 2.84 (s, 6H), 4.75 (s, 1H), 6.99 (m, 1H), 7.09 (m, 1H), 7.19 (m, 1H), 7.35 (m, 3H), 7.46 (m, 4H), 7.75 (m, 2H); MS (ES+) 346/347.

### Example 67

### 5-phenyl-2-pyrrolidin-1-yl-3-thien-2-yl-3H-1,4-benzodiazepine

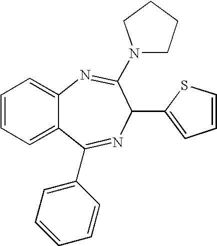

Using an analogous procedure to that described in Example 50 using steps 4 to 8, the titled compound was formed from (2-aminophenyl)(phenyl)methanone except that pyrrolidine was used in step 8. ¹H NMR (300, CDCl₃) δ 1.70 (m, 4H), 2.03 (m, 2H), 3.56 (m, 1H), 3.85 (m, 1H), 4.78 (s, 1H), 6.94 (m, 1H), 7.10 (m, 1H), 7.19 (m, 1H), 7.35 (m, 3H), 7.46 (m, 4H), 7.75 (d, 2H, J=7.44); MS (ES+) 372/373.

### Example 68

### 2-azetidin-1-yl-5-phenyl-3-thien-2-yl-3H-1,4-benzodiazepine

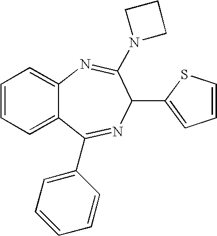

Using an analogous procedure to that described in Example 50 using steps 4 to 8, the titled compound was formed from (2-aminophenyl)(phenyl)methanone except that azetidine was used in step 8. ¹H NMR (300, CDCl₃) δ2.07 (m, 2H), 3.37 (m, 1H), 3.81 (m, 1H), 3.96 (m, 1H), 4.16 (m, 1H), 4.72 (s, 1H), 6.97 (m, 1H), 7.12 (m, 1H), 7.22 (m, 1H), 7.36 (m, 2H), 7.46 (m, 5H), 7.72 (m, 2H); MS (ES+) 358/359.

### Example 69

### 2-(4,5-dihydro-1H-pyrazol-1-yl)-5-phenyl-3-thien-2-yl-3H-1,4-benzodiazepine

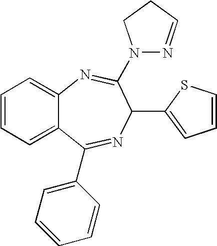

Using an analogous procedure to that described in Example 50 using steps 4 to 8, the titled compound was formed from (2-aminophenyl)(phenyl)methanone except that 4,5-dihydro-1H-pyrazole was used in step 8. ¹H NMR (300, CDCl₃) δ 1.58 (m, 2H), 4.13 (m, 2H), 4.64 (m, 1H), 4.82 (s, 1H), 6.97 (m, 1H), 7.19 (m, 2H), 7.35 (m, 2H), 7.44 (m, 5H), 7.69 (d, 2H, J=6.78); MS (ES+) 371/372.

### Example 70

### 2-[(5-phenyl-3-thien-2-yl-3H-1,4-benzodiazepin-2-yl)amino]ethanol

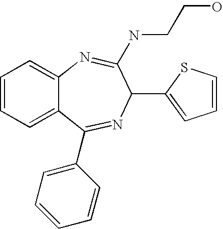

Using an analogous procedure to that described in Example 50 using steps 4 to 8, the titled compound was formed from (2-aminophenyl)phenyl)methanone except that ethanolamine was used in step 8. ¹H NMR (300, CDCl₃) δ 2.98 (s, 1H), 3.29 (m, 1H), 3.64 (m, 1H), 3.79 (m, 2H), 4.91 (s, 1H), 5.44 (br s, 1H), 7.07 (m, 1H), 7.20 (m, 1H), 7.24 (m, 1H), 7.36-7.51 (m, 7H), 7.69 (m, 2H); MS (ES+) 362/363.

### Example 71

### N-(2-methoxyethyl)-5-phenyl-3-thien-2-yl-3H-1,4-benzodiazepin-2-amine

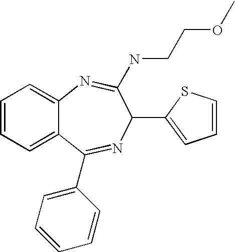

Using an analogous procedure to that described in Example 50 using steps 4 to 8, the titled compound was formed from (2-aminophenyl)(phenyl)methanone except that 2-methoxyethanamine was used in step 8. ¹H NMR (300, CDCl₃) δ 3.26 (m, 2H), 3.27 (s, 3H), 3.45 (m, 1H), 3.52 (m, 1H), 4.86 (s, 1H), 5.25 (br s, 1H), 6.99 (m, 1H), 7.19 (m, 2H), 7.44 (m, 7H), 7.70 (m, 2H); MS (ES+) 376/377.

### Example 72

### 1-(5-phenyl-3-thien-2-yl-3H-1,4-benzodiazepin-2-yl)pyrrolidin-3-ol

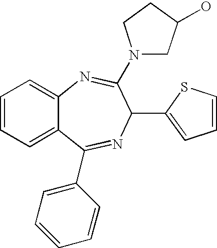

Using an analogous procedure to that described in Example 50 using steps 4 to 8, the titled compound was formed from (2-aminophenyl)(phenyl)methanone except that pyrrolidin-3-ol was used in step 8. ¹H NMR (300, CDCl₃) δ 1.76 (m, 2H), 3.05 (m, 1H), 3.26 (m, 1H), 4.03 (m, 1H), 4.23 (m, 1H), 4.77 (s, 1H), 4.85 (m, 1H), 6.98 (m, 1H), 7.09 (m, 1H), 7.21 (m, 1H), 7.36 (m, 3H), 7.46 (m, 4H), 7.72 (m, 2H); MS (ES+) 388/389.

### Example 73

### 1-(5-phenyl-3-thien-2-yl-3H-1,4-benzodiazepin-2-yl)azetidin-3-ol

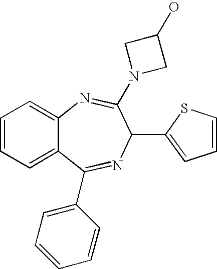

Using an analogous procedure to that described in Example 50 using steps 4 to 8, the titled compound was formed from (2-aminophenyl)(phenyl)methanone except that azetidin-3-ol was used in step 8. ¹H NMR (300, DMSO-d₆) δ 3.30 (m, 4H), 4.22 (m, 1H), 5.47 (m, 1H), 7.02 (m, 1H), 7.15 (m, 1H), 7.25 (m, 2H), 7.51 (m, 6H), 7.59 (m, 2H); MS (ES+) 374.

### Example 74

### N-methyl-5-phenyl-3-(2-thienyl)-3H-pyrido[2,3-e][1,4]diazepin-2-amine

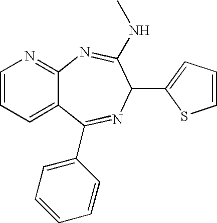

Using an analogous procedure to that described in Example 50, the titled compound was formed from benzaldehyde. ¹H NMR (300 MHz, CHLOROFORM-d): δ ppm 2.94 (d, J=4.75 Hz, 3H); 4.90 (s, 1H); 5.13 (s, br, 1H); 6.95 (m, 1H); 7.16 (m, 2H); 7.45 (m, 4H); 7.66 (m, 3H); 8.68 (m, 1H). ES (MH⁺)⁺=333.

### Example 75

### N-ethyl-5-phenyl-3-thien-2-yl-3H-pyrido[2,3-e][1,4]diazepin-2-amine

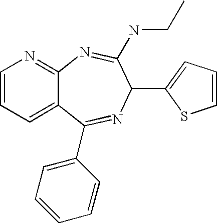

Using an analogous procedure to that described in Example 50, the titled compound was formed from benzaldehyde except that ethylamine was used in step 8. ¹H NMR (300, CDCl₃) δ 1.11 (t, J=7.25, 3H), 3.35-3.44 (m, 1H), 3.48-3.59 (m, 1H), 4.90 (s, 1H), 5.07 (br s, 1H), 6.97 (m, 1H), 7.21 (m, 2H), 7.47 (m, 4H), 7.68 (m, 3H), 8.71 (dd, J=1.88, 4.71, 1H); MS (ES+) 347.

### Example 76

### N-(2-fluoroethyl)-5-phenyl-3-thien-2-yl-3H-pyrido[2,3-e][1,4]diazepin-2-amine

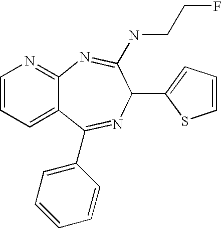

Using an analogous procedure to that described in Example 50, the titled compound was formed from benzaldehyde except that 2-fluoroethylamine was used in step 8. ¹H NMR (300, DMSO-d₆) δ 3.46-3.61 (m, 2H) 4.33-4.63 (m, 2H) 4.83 (s, 1H) 6.39 (m, 1H) 7.04 (m, 1H) 7.15-7.28 (m, 2H) 7.45-7.60 (m, 5H) 7.62-7.69 (m, 2H) 8.62 (m, 1H); MS (ES+) 365.

### Example 77

### 5-(4-chlorophenyl)-N-methyl-3-(2-thienyl)-3H-1,4-benzodiazepin-2-amine

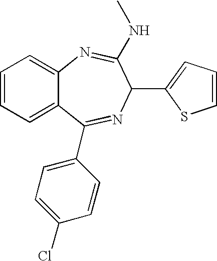

Using an analogous procedure to that described in Example 50 using steps 4 to 8, the titled compound was formed from (2-aminophenyl)(4-chlorophenyl)methanone. ¹H NMR (300 MHz, CHLOROFORM-d): δ ppm 2.87 (d, J=4.74 Hz, 3H); 4.86 (s, 1H); 4.91 (s, br, 1H); 6.9-7.60 (m, 11H). ES (MH⁺)⁺=366

### Example 78

### 7-chloro-N-methyl-5-pyridin-3-yl-3-(2-thienyl)-3H-1,4-benzodiazepin-2-amine

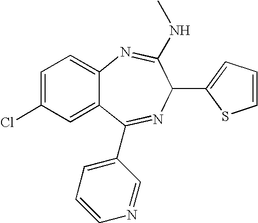

Using an analogous procedure to that described in Example 50 using steps 4 to 8, the titled compound was formed from (2-amino-5-chlorophenyl)(pyridin-3-yl)methanone. ¹H NMR (300 MHz, CHLOROFORM-d): δ ppm 2.88 (d, J=4.72 Hz, 3H); 4.85 (s, 1H); 4.98 (s, br 1H); 7.0-7.5 (m, 7H); 7.99 (d, J=7.83 Hz, 1H); 8.72 (d, J=4.30 Hz, 1H); 8.92 (s, 1H). ES (MH⁺)⁺=367

### Example 79

### 7-chloro-N-methyl-3,5-di-2-thienyl-3H-1,4-benzodiazepin-2-amine

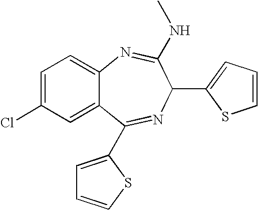

Using an analogous procedure to that described in Example 50 using steps 4 to 8, the titled compound was formed from (2-amino-5-chlorophenyl)(thien-2-yl)methanone. ¹H NMR (300 MHz, CHLOROFORM-d): δ ppm 2.86 (d, J=4.81 Hz, 3H); 4.86 (s, 1H); 4.93 (s, br, 1H); 7.0-7.3 (m, 5H); 7.45 (m, 3H); 7.66 (d, J=2.35 Hz, 1H). ES (MH⁺)⁺=372

### Example 80

### 5-phenyl-3-(2-thienyl)-N-(2,2,2-trifluoroethyl)-3H-pyrido[2,3-e][1,4]diazepin-2-amine

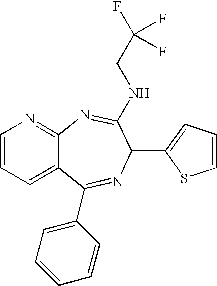

Using an analogous procedure to that described in Example 50, the titled compound was formed from benzaldehyde except that 2,2,2-trifluoroethanamine was used in step 8. ¹H NMR (300 MHz, CHLOROFORM-d): δ ppm 4.11 (m, 2H); 4.93 (s, 1H); 5.28 (s, br, 1H); 7.04 (m, 1H); 7.20 (m, 2H); 7.49 (m, 4H); 7.65 (d, J=7.01 Hz, 2H); 7.77 (d, J=7.76 Hz, 1H); 8.76 (d, J=1.51 Hz, 1H). ES (MH⁺)⁺−401

### Example 81

### N-(2,2-difluoroethyl)-5-phenyl-3-(2-thienyl)-3H-pyrido[2,3-e][1,4]diazepin-2-amine

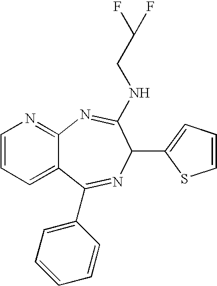

Using an analogous procedure to that described in Example 50, the titled compound was formed from benzaldehyde except that 2,2,-difluoroethanamine was used in step 8. ¹H NMR (300 MHz, CHLOROFORM-d): δ ppm 3.78 (m, 1H); 4.05 (m, 1H); 5.06 (s, 1H); 5.93 (s, br, 1H); 7.2-7.8 (m, 10H); 8.05 (d, J=5.19 Hz, 1H); 9.13 (s, 1H). ES (MH⁺)⁺=383

### Example 82

### 1-[5-phenyl-3-(2-thienyl)-3H-pyrido[2,3-e][1,4]diazepin-2-yl]azetidin-3-ol

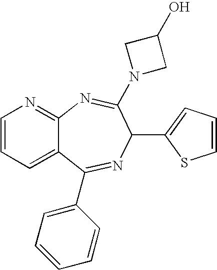

Using an analogous procedure to that described in Example 50, the titled compound was formed from benzaldehyde except that azetidin-3-ol was used in step 8. ¹H NMR (300 MHz, CHLOROFORM-d): δ ppm 3.2-4.5 (m, 5H); 4.85 (s, 1H); 6.85 (m, 1H); 7.03 (s, 1H); 7.2-7.8 (m, 9H); 8.59 (s, 1H). ES (MH⁺)⁺=375

### Example 83

### 1-[5-phenyl-3-(2-thienyl)-3H-pyrido[2,3-e][1,4]diazepin-2-yl]azetidin-3-amine

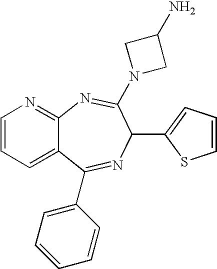

Using an analogous procedure to that described in Example 50, the titled compound was formed from benzaldehyde except that tert-butyl azetidin-3-ylcarbamate was used in step 8 followed by BOC deprotection with TFA/CH₂Cl₂. ¹H NMR (300 MHz, CHLOROFORM-d): δ ppm 2.40 (s, br, 2H); 3.2-4.6 (m, 5H); 4.70 (s, 1H); 6.85 (s, 1H); 7.0 (m, 1H); 7.20 (s, 1H); 7.3-7.5 (m, 4H); 7.63 (m, 3H); 8.55 (s, 1H). ES (MH⁺)⁺=374

### Example 84

### N-cyclopropyl-3-(4-fluorophenyl)-5-(2-thienyl)-3H-pyrido[2,3-e][1,4]diazepin-2-amine

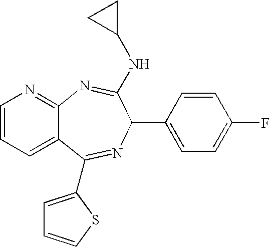

### a) (2-aminopyridin-3-yl)(2-thienyl)methanone

A mixture of (2-chloropyridin-3-yl)(2-thienyl)methanone (3.0 g), 28% aqueous ammonium hydroxide (50 mL), and 2M ammonia in methanol (50 mL) was heated in a sealed reactor at 150° C. for 5 hrs. Reaction was allowed to cool to room temperature. Solvent methanol was removed under reduced pressure, and extracted twice with ethyl acetate. The combined organic extracts were dried over anhydrous sodium sulfate, filtered, and evaporated under reduced pressure. The residue was purified by column chromatography over silica eluting with 30-40% ethyl acetate in hexanes to give the subtitle compound (2.2 g). Mass: 205.2 (M+H).

### b) [(tert-butoxycarbonyl)amino](4-fluorophenyl)acetic acid

This compound was synthesized following the procedure described in the literature (J. Org. Chem., 62, 1997, 1553-1555) starting from amino(4-fluorophenyl)acetic acid and di-tert-butyl dicarbonate. Mass: 268.3 (M−H).

### c) tert-butyl (1-(4-fluorophenyl)-2-oxo-2-{[3-(2-thienylcarbonyl)pyridin-2-yl]amino}ethyl)carbamate

1,3-Dicyclohexylcarbodiimide (1.85 g) was added as a solid to a mixture of (2-aminopyridin-3-yl)(2-thienyl)methanone (0.612 g) and [(tert-butoxycarbonyl)amino](4-fluorophenyl)acetic acid (1.614 g) in dichloromethane (30 mL). The reaction was stirred at room temperature for 3 days. N,N′-dicyclohexylurea precipitated from the reaction was filtered and washed with dichloromethane. Combined filtrates were concentrated under reduced pressure. The residue was purified by column chromatography over silica eluting with 30-45% ethyl acetate in hexanes to give the subtitle compound (0.631 g). Mass: 456.1 (M+H).

d) 3-(4-fluorophenyl)-5-(2-thienyl)-1,3-dihydro-2H-pyrido[2,3-e][1,4]diazepin-2-one tert-butyl (1-(4-fluorophenyl)-2-oxo-2-{[3-(2-thienylcarbonyl)pyridin-2-yl]amino}ethyl)carbamate (0.415 g) was treated with 30% TFA in dichloromethane (30 mL) at room temperature for one hour. Solvent methanol and TFA were removed under reduced pressure. The residue was dissolved in acetic acid (10 mL) and treated with ammonium acetate (2 g) at room temperature for 3 days. Reaction was diluted with water and extracted with ethyl acetate. The combined organic extracts were dried over anhydrous sodium sulfate, filtered, and evaporated under reduced pressure to give the subtitle compound (0.285 g). Mass: 338.1 (M+H).

e) N-cyclopropyl-3-(4-fluorophenyl)-5-(2-thienyl)-3H-pyrido[2,3-e][1,4]diazepin-2-amine Titanium tetrachloride (0.44 mL, 1M solution in dichloromethane) was added to a mixture of 3-(4-fluorophenyl)-5-(2-thienyl)-1,3-dihydro-2H-pyrido[2,3-e][1,4]diazepin-2-one (0.05 g) and cyclopropylamine (0.1 mL) in dry THF (3.5 mL). The mixture was heated in microwave reactor at 80° C. for 20 minutes. Reaction was cooled and poured into 5% aqueous sodium bicarbonate solution. Inorganic salts separated were filtered through celite and washed with ethyl acetate. Organic layer separated and aqueous layer was further extracted with ethyl acetate. The combined organic layers were dried over anhydrous sodium sulfate, filtered, and evaporated under reduced pressure. The residue was purified by column chromatography over silica eluting with 50-80% ethyl acetate in hexanes to give the subtitle compound (0.0325 g). Mass: 377.1 (M+H). ¹H NMR (400 MHz, DMSO-D6) δ 0.18-0.41 (m, 1H) 0.42-0.72 (m, 3H) 2.78 (d, J=3.91 Hz, 1H) 4.54 (s, 1H) 5.99 (s, 1H) 7.01-7.24 (m, 3H) 7.29 (t, J=8.32 Hz, 2H) 7.63-7.88 (m, 3H) 8.06 (d, J=7.83 Hz, 1H) 8.63 (d, J=4.89 Hz, 1H)

### Example 85

### N-cyclopropyl-3-(3-fluorophenyl)-5-(2-thienyl)-3H-pyrido[2,3-e][1,4]diazepin-2-amine

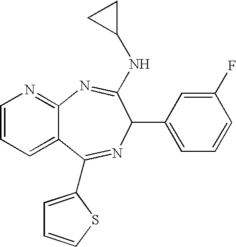

This compound was synthesized following reaction sequence described above for the example 84 starting from (2-aminopyridin-3-yl)(2-thienyl)methanone and [(tert-butoxycarbonyl)amino](3-fluorophenyl)acetic acid. Mass: 377.1 (M+H). ¹H NMR (400 MHz, DMSO-D6) δ 0.15-0.34 (m, 1H) 0.34-0.64 (m, 3H) 2.72 (s, 1H) 4.49 (s, 1H) 5.99 (d, J=3.91 Hz, 1H) 6.96-7.24 (m, 4H) 7.30-7.55 (m, 3H) 7.70 (d, J=4.89 Hz, 1H) 7.99 (d, J=7.83 Hz, 1H) 8.57 (d, J=3.91 Hz, 1H)

### Example 86

### N-cyclopropyl-3,5-di-2-thienyl-3H-pyrido[2,3-e][1,4]diazepin-2-amine

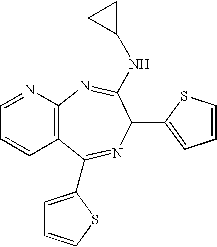

This compound was synthesized following the procedure described in the example 84e using 3,5-di-2-thienyl-1,3-dihydro-2H-pyrido[2,3-e][1,4]diazepin-2-one and cyclopropylamine. Mass: 365.11 (M+H). ¹H NMR (400 MHz, DMSO-D6) δ 0.29-0.44 (m, 1H) 0.46-0.71 (m, 3H) 2.81 (s, 1H) 4.79 (s, 1H) 6.13 (d, J=3.91 Hz, 1H) 7.03-7.27 (m, 5H) 7.60 (d, J=4.89 Hz, 1H) 7.78 (d, J=4.89 Hz, 1H) 8.05 (d, J=8.80 Hz, 1H) 8.63 (d, J=4.89 Hz, 1H)

### Example 87

### N-prop-2-yn-1-yl-3,5-di-2-thienyl-3H-pyrido[2,3-e][1,4]diazepin-2-amine

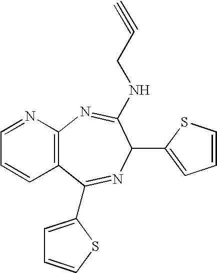

This compound was synthesized following the procedure described in the example 84e using 3,5-di-2-thienyl-1,3-dihydro-2H-pyrido[2,3-e][1,4]diazepin-2-one and prop-2-yn-1-amine. Mass: 363.06 (M+H). ¹H NMR (400 MHz, DMSO-D6) δ 3.00 (s, 1H) 3.89-4.00 (m, 2H) 4.76-4.98 (m, 1H) 6.63 (t, J=5.87 Hz, 1H) 7.03-7.30 (m, 5H) 7.64 (d, J=4.89 Hz, 1H) 7.78 (d, J=4.89 Hz, 1H) 8.07 (d, J=7.83 Hz, 1H) 8.64 (s, 1H)

### Example 88

### 2-azetidin-1-yl-3,5-di-2-thienyl-3H-pyrido[2,3-e][1,4]diazepine

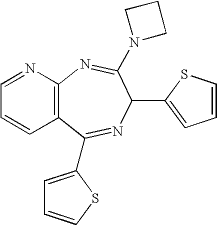

This compound was synthesized following the procedure described in the example 84e using 3,5-di-2-thienyl-1,3-dihydro-2H-pyrido[2,3-e][1,4]diazepin-2-one and azetidine. Mass: 365.1 (M+H). ¹H NMR (400 MHz, DMSO-D6) δ 1.91-2.17 (m, 2H) 3.20-3.40 (m, 1H) 3.50-3.72 (m, 1H) 3.76-3.96 (m, 1H) 3.97-4.17 (m, 1H) 4.74 (s, 1H) 7.00-7.23 (m, 4H) 7.28 (d, J=3.91 Hz, 1H) 7.58 (d, J=4.89 Hz, 1H) 7.80 (d, J=4.89 Hz, 1H) 8.07 (d, J=7.83 Hz, 1H) 8.62 (s, 1H)

### Example 89

### N-ethyl-3,5-di-2-thienyl-3H-pyrido[2,3-e][1,4]diazepin-2-amine

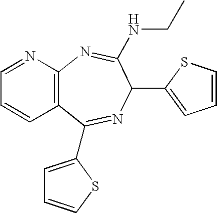

This compound was synthesized following the procedure described in the example 84e using 3,5-di-2-thienyl-1,3-dihydro-2H-pyrido[2,3-e][1,4]diazepin-2-one and ethylamine. Mass: 353.1 (M+H). ¹H NMR (300 MHz, DMSO-D6) δ 1.01 (t, J=7.16 Hz, 3H) 3.24 (q, J=7.16 Hz, 2H) 4.83 (s, 1H) 6.27 (s, 1H) 7.08 (dd, J=7.72, 4.71 Hz, 1H) 7.13-7.28 (m, 4H) 7.63 (d, J=4.90 Hz, 1H) 7.78 (d, J=4.14 Hz, 1H) 7.97-8.13 (m, 1H) 8.55-8.66 (m, 1H)

### Example 90

### 5-(2-furyl)-N-methyl-3-(1-methyl-1H-pyrrol-2-yl)-3H-pyrido[2,3-e][1,4]diazepin-2-amine

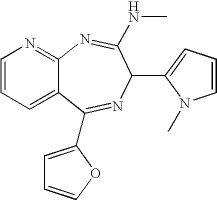

a) 5-(2-furyl)-3-(1-methyl-1H-pyrrol-2-yl)-1,3-dihydro-2H-pyrido[2,3-e][1,4]diazepin-2-one A mixture of 3-[2-furyl(imino)methyl]pyridin-2-amine (0.374 g) and ethyl amino(1-methyl-1H-pyrrol-2-yl)acetate (0.364 g) in methanol (5 mL) was heated at 90° C. for 30 minutes in microwave reactor. Reaction was cooled to room temperature, and 1,8-diazabicyclo[5.4.0]undec-7-ene (0.912 mL) was added. Then the reaction was again heated in microwave oven at 70° C. for 25 minutes. Solvent methanol was removed under reduced pressure, quenched with 5% aqueous ammonium chloride solution, and extracted with ethyl acetate. The combined organic extracts were dried over anhydrous sodium sulfate, filtered, and evaporated under reduced pressure. The residue was triturated with methanol. The solid product filtered, and dried to give the subtitle compound (0.24 g). Mass: 307.04 (M+H). Mother liquor also contains considerable amount of required product that can be further purified.

ethyl amino(1-methyl-1H-pyrrol-2-yl)acetate was prepared as follows: Pyridine (20 mL, 0.45 mol) in dry CH₂Cl₂ (400 mL) was added drop wise to a solution of chloroethyl oxalate in CH₂Cl₂ (400 mL) at −20° C. to −30° C. under nitrogen atmosphere followed by a dropwise addition of N-methylpyrrole (20.0 mL, 0.23 mol) in CH₂Cl₂ (300 mL). The reaction mixture was stirred at −20° C. to −30° C. for 4 h, and overnight at 0° C. The mixture was washed with 0.25 M aq. HCl (3×100 mL), water (2×100 mL), brine (2×100 mL), dried (MgSO₄), filtered, and concentrated to give ethyl (1-methyl-1H-pyrrol-2-yl)(oxo)acetate (40.0 g, near quantitative yield, 95% pure by H NMR) as a golden yellow oil.

A mixture of compound ethyl (1-methyl-1H-pyrrol-2-yl)(oxo)acetate (40.0 g, 0.23 mol), NH₂OH.HCl (33.5 g, 0.48 mol), and pyridine (39.0 mL, 0.48 mol) in absolute ethanol was refluxed for 4 h. The reaction mixture was concentrated to ⅓ volume and extracted with ether (3×100 mL). The combined organic extracts were washed with water (2×50 mL), brine (2×50 mL), dried (MgSO₄), filtered and concentrated. The crude product was purified by flash chromatography using gradient of 20%, 22%, 25% EtOAc/hexanes as eluent to give ethyl (2Z)-(hydroxyimino)(1-methyl-1H-pyrrol-2-yl)acetate (20.7 g, 44% yield) as a yellow solid.

Aluminum foil [4.7 g, 0.15 mol, cut as strips (˜1×2.5 cm) and then rolled into cylinders] was amalgamated in 2% aq. HgCl₂ solution for 120 seconds and decanted. The resulting amalgam was rinsed in absolute ethanol followed by THF, and was placed in a solution of THF:water (10:1, 770 mL). A solution of ethyl (2Z)-(hydroxyimino)(1-methyl-1H-pyrrol-2-yl)acetate in THF (50 mL) was added quickly and the resulting mixture was heated to reflux for 3 h. The reaction mixture was filtered through Celite and washed with EtOAc. The solid waste was disposed separately as mercury waste. The filtrate as concentrated to give the crude product. Purification by flash chromatography using CH₂Cl₂ and ether as eluent gave amine as a yellow oil (8.9 g). It was dissolved in dry ether and was treated with 2M HCl in ether at 0° C. to give a mono-HCl salt of ethyl amino(1-methyl-1H-pyrrol-2-yl)acetate (10.51 g, 45% yield) as a white solid after concentration.

b) 5-(2-furyl)-N-methyl-3-(1-methyl-1H-pyrrol-2-yl)-3H-pyrido[2,3-e][1,4]diazepin-2-amine This compound was synthesized following the procedure described in the example 84e using 5-(2-furyl)-3-(1-methyl-1H-pyrrol-2-yl)-1,3-dihydro-2H-pyrido[2,3-e][1,4]diazepin-2-one and methylamine. Mass: 320.09 (M+H). ¹H NMR (300 MHz, DMSO-D6) δ 2.72 (d, J=4.52 Hz, 3H) 3.43 (s, 3H) 4.55 (s, 1H) 6.01-6.19 (m, 2H) 6.53 (s, 1H) 6.66 (s, 1H) 6.83 (d, J=3.01 Hz, 2H) 7.05 (dd, J=7.91, 4.90 Hz, 1H) 7.81-8.07 (m, 2H) 8.58 (d, J=3.01 Hz, 1H)

### Example 91

### N-methyl-3-(1-methyl-1H-pyrrol-2-yl)-5-(2-thienyl)-3H-pyrido[2,3-e][1,4]diazepin-2-amine

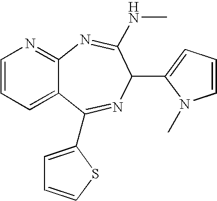

This compound was synthesized following reaction sequence described above for the Example 90 starting from 3-[imino(2-thienyl)methyl]pyridin-2-amine and ethyl amino(1-methyl-1H-pyrrol-2-yl)acetate. Mass: 336.08 (M+H). ¹H NMR (300 MHz, DMSO-D6) δ 2.65 (d, J=4.52 Hz, 3H) 3.38 (s, 3H) 4.51 (s, 1H) 5.97-6.14 (m, 2H) 6.42 (s, 1H) 6.77 (s, 1H) 7.00 (dd, J=7.54, 4.52 Hz, 1H) 7.05-7.13 (m, 2H) 7.62-7.73 (m, 1H) 7.96 (d, J=6.03 Hz, 1H) 8.52 (d, J=4.52 Hz, 1H)

### Example 92

### 5-(4-fluorophenyl)-N-methyl-3-thien-2-yl-3H-pyrido[2,3-e][1,4]diazepin-2-amine

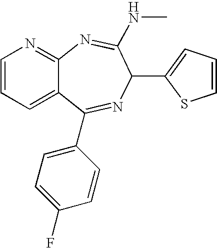

### a) (4-fluorophenyl)(2-fluoropyridin-3-yl)methanol

To a solution of 2-fluoropyridine (4.4 mL) in THF (40 mL) was cooled to −78° C. Lithium diisopropyl amide (26 mL) was then added dropwise. The reaction was stirred at −78° C. for 1 hr after which 4-fluorobenzaldehyde (5.4 mL) in 20 mL of THF was added dropwise to the reaction. The reaction was allowed to slowly warm up to room temperature and stirred for 2.5 hrs. The reaction was quenched by the addition of water and THF was removed under reduced pressure. The residue was poured into ethyl acetate and washed with water. Extraction was done three times with ethyl acetate. The combined organic extracts were washed with brine and then dried with Na₂SO₄ and filtered. Ethyl acetate was evaporated under reduced pressure and the residue was dried under the house vacuum to give the subtitled compound (8.28 g). Mass: 222.3 (M+H).

### b) (4-fluorophenyl)(2-fluoropyridin-3-yl)methanone

To a solution of (4-fluorophenyl)(2-fluoropyridin-3-yl)methanol (8.28 g) in dichloromethane (40 mL) was added PCC (9 g). The reaction was stirred at room temperature for 3 hrs. The mixture was filtered through a bed of Celite and later washed with water. Extraction was done three times with dichloromethane. The combined organic extracts were dried with Na₂SO₄ and filtered. Dichloromethane was then removed under reduced pressure. The residue was dried under the house vacuum to give the subtitled compound (6.62 g). Mass: 220.2 (M+H).

### c) (2-aminopyridin-3-yl)(4-fluorophenyl)methanone

To a solution of (4-fluorophenyl)(2-fluoropyridin-3-yl)methanone (6.62 g) in methanol (100 mL) was added 7M ammonia in methanol (40 mL). The reaction was stirred at 100° C. in a pressure reactor (bomb) overnight. After allowing the reaction to cool to room temperature, methanol was removed under reduced pressure. The residue was then redissolved in ethyl acetate and washed with water. Extraction was done three times with ethyl acetate. The combined organic extracts were dried with Na₂SO₄ and filtered. Ethyl acetate was removed under reduced pressure and the residue was purified by column chromatography over silica eluting with hexanes:ethyl acetate (3:2) to give the subtitled compound (3.36 g). Mass: 217.2 (M+H)

### d) tert-butyl 2-{[3-(4-fluorobenzoyl)pyridin-2-yl]amino}-2-oxo-1-thien-2-ylethylcarbamate

To a solution of (2-aminopyridin-3-yl)(4-fluorophenyl)methanone (1.1 g) in dichloromethane (40 mL) was added [(tert-butoxycarbonyl)amino](thien-2-yl)acetic acid (2 g). The reaction was stirred at room temperature and DCC (2.6 g) in dichloromethane (20 mL) was added fast dropwise. The reaction was allowed to stir overnight at room temperature. The next day, the residue was filtered and washed with water. Extraction was done three times with dichloromethane. The combined organic extracts were dried with Na₂SO₄ and dichloromethane was removed under reduced pressure. The residue was purified by column chromatography over silica eluting with hexanes:ethyl acetate (3:2) to give the subtitled compound (1.63 g). Mass: 456.1 (M+H)

### e) 5-(4-fluorophenyl)-3-thien-2-yl-1,3-dihydro-2H-pyrido[2,3-e][1,4]diazepin-2-one

A solution of tert-butyl 2-{[3-(4-fluorobenzoyl)pyridin-2-yl]amino}-2-oxo-1-thien-2-ylethylcarbamate (1.1 g) in dichloromethane (35 mL) was cooled in an ice bath to 0° C. TFA (15 mL) was then added fast dropwise while the reaction was stirring at 0° C. The reaction was allowed to warm to room temperature and stirred for 2 hrs. After 2 hrs, the solvent system was removed under reduced pressure and the residue was dried under the house vacuum for about 2 hrs. To the residue was added acetic acid (30 mL) and ammonium acetate (6 g). The reaction was stirred at room temperature overnight. The next day, the reaction was washed with water and extracted with dichloromethane. The combined organic extracts were dried with Na₂SO₄ and dichloromethane was removed under reduced pressure. An attempt was made to purify the residue by column chromatography over silica eluting with hexanes:ethyl acetate:methanol (16:3:1) which was unsuccessful. The subtitled compound was obtained with some impurities (340 mg). Mass: 338.1 (M+H).

### f) 5-(4-fluorophenyl)-N-methyl-3-thien-2-yl-3H-pyrido[2,3-e][1,4]diazepin-2-amine

To 5-(4-fluorophenyl)-3-thien-2-yl-1,3-dihydro-2H-pyrido[2,3-e][1,4]diazepin-2-one (27 mg) was added 1M methylamine in THF (1 mL). Titanium (IV) chloride solution in dichloromethane 1M (0.3 mL) was then added to the reaction. The mixture was heated for 5 minutes at 80° C. in the microwave reactor. The reaction was cooled then poured into a solution of sodium bicarbonate and ethyl acetate. The mixture was then filtered through a bed of celite and washed with ethyl acetate. Organic layer was separated and the aqueous layer was further extracted with ethyl acetate. The combined organic extracts were dried with Na₂SO₄ and ethyl acetate was removed under reduced pressure. The residue was purified by column chromatography over silica eluting with ethyl acetate to give the subtitled compound (2.8 mg). ¹H NMR (300 MHz, DMSO-D6) δ ppm 2.72 (d, J=4.33 Hz, 3H) 4.82 (s, 1H) 6.46 (s, 1H) 7.04 (s, 1H) 7.15-7.21 (m, 1H) 7.23 (s, 1H) 7.33 (t, J=8.85 Hz, 2H) 7.58-7.72 (m, 4H) 8.62 (s, 1H). Mass: 351.1 (M+H)

### Example 93

### N-cyclopropyl-5-(4-fluorophenyl)-3-thien-2-yl-3H-pyrido[2,3-e][1,4]diazepin-2-amine

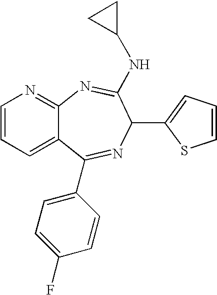

The subtitled compound was synthesized following reaction described above for the example 92f starting with 5-(4-fluorophenyl)-3-thien-2-yl-1,3-dihydro-2H-pyrido[2,3-e][1,4]diazepin-2-one and cyclopropylamine. ¹H NMR (300 MHz, DMSO-D6) δ ppm 0.18-0.35 (m, 1H) 0.38-0.63 (m, 3H) 2.67-2.81 (m, 1H) 4.67 (s, 1H) 6.08 (d, J=3.77 Hz, 1H) 6.97 (dd, J=7.63, 4.80 Hz, 1H) 7.05-7.12 (m, 1H) 7.15 (d, J=3.01 Hz, 1H) 7.20-7.33 (m, 2H) 7.41-7.78 (m, 4H) 8.55 (d, J=3.01 Hz, 1H). Mass: 377.1 (M+H).

### Example 94

### N-ethyl-5-(4-fluorophenyl)-3-thien-2-yl-3H-pyrido[2,3-e][1,4]diazepin-2-amine

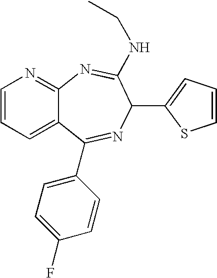

The subtitled compound was synthesized following reaction described above for the example 92f starting with 5-(4-fluorophenyl)-3-thien-2-yl-1,3-dihydro-2H-pyrido[2,3-e][1,4]diazepin-2-one and ethylamine. ¹H NMR (300 MHz, DMSO-D6) δ ppm 0.92 (t, J=7.06 Hz, 3H) 3.06-3.22 (m, 2H) 4.70 (s, 1H) 6.19 (t, J=5.46 Hz, 1H) 6.94 (dd, J=7.82, 4.62 Hz, 1H) 7.06-7.14 (m, 1H) 7.16 (d, J=3.39 Hz, 1H) 7.25 (t, J=8.76 Hz, 2H) 7.39-7.82 (m, 4H) 8.28-8.74 (m, 1H). Mass: 365.1 (M+H).

### Example 95

### 5-(3-fluorophenyl)-N-methyl-3-thien-2-yl-3H-pyrido[2,3-e][1,4]diazepin-2-amine

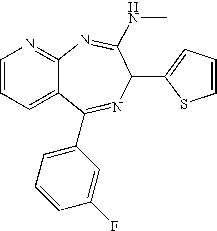

The subtitled compound was synthesized following reaction sequence described above for the example 92 starting with 2-fluoropyridine and 3-fluorobenzaldehyde. ¹H NMR (300 MHz, DMSO-D6) δ ppm 2.65 (d, J=4.52 Hz, 3H) 4.74 (s, 1H) 6.33 (d, J=4.71 Hz, 1H) 6.95 (dd, J=7.72, 4.71 Hz, 1H) 7.06-7.14 (m, 1H) 7.13-7.20 (m, 1H) 7.22-7.38 (m, 3H) 7.41-7.51 (m, 1H) 7.56 (d, J=4.90 Hz, 1H) 7.62 (dd, J=7.72, 1.51 Hz, 1H) 8.54 (d, J=2.83 Hz, 1H). Mass: 351.1 (M+H).

### Example 96

### N-cyclopropyl-5-(3-fluorophenyl)-3-thien-2-yl-3H-pyrido[2,3-e][1,4]diazepin-2-amine

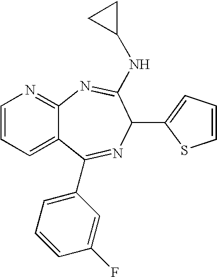

The subtitled compound was synthesized following reaction described above for the example 92f starting with 5-(3-fluorophenyl)-3-thien-2-yl-1,3-dihydro-2H-pyrido[2,3-e][1,4]diazepin-2-one and cyclopropylamine. ¹H NMR (300 MHz, DMSO-D6) δ ppm 0.14-0.35 (m, 1H) 0.36-0.68 (m, 3H) 2.62-2.89 (m, 1H) 4.70 (s, 1H) 6.11 (d, J=3.77 Hz, 1H) 6.98 (dd, J=7.82, 4.62 Hz, 1H) 7.09 (dd, J=4.90, 3.58 Hz, 1H) 7.12-7.19 (m, 1H) 7.24-7.39 (m, 3H) 7.42-7.52 (m, 1H) 7.52-7.58 (m, 1H) 7.63 (dd, J=7.91, 1.88 Hz, 1H) 8.56 (dd, J=4.52, 1.88 Hz, 1H). Mass: 377.1 (M+H).

### Example 97

### N-ethyl-5-(3-fluorophenyl)-3-thien-2-yl-3H-pyrido[2,3-e][1,4]diazepin-2-amine

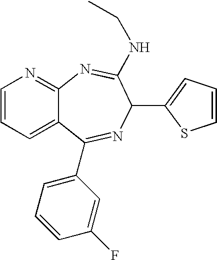

The subtitled compound was synthesized following reaction described above for the example 92f starting with 5-(3-fluorophenyl)-3-thien-2-yl-1,3-dihydro-2H-pyrido[2,3-e][1,4]diazepin-2-one and ethylamine. ¹H NMR (300 MHz, DMSO-D6) δ ppm 0.93 (t, J=7.06 Hz, 3H) 3.08-3.22 (m, 2H) 4.74 (s, 1H) 6.25 (s, 1H) 6.95 (dd, J=7.82, 4.62 Hz, 1H) 7.12 (dd, J=5.09, 3.58 Hz, 1H) 7.18 (d, J=3.58 Hz, 1H) 7.23-7.40 (m, 3H) 7.46 (d, J=14.69 Hz, 1H) 7.53-7.69 (m, 2H) 8.54 (dd, J=4.62, 1.98 Hz, 1H). Mass: 365.1 (M+H).

### Example 98

### N-methyl-5-pyridin-3-yl-3-thien-2-yl-3H-pyrido[2,3-e][1,4]diazepin-2-amine

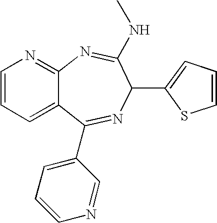

The subtitled compound was synthesized following reaction sequence described above for the example 92 starting 2-fluoro pyridine and 3-pyridine carboxaldehyde. ¹H NMR (300 MHz, DMSO-D6) δ ppm 2.65 (d, J=4.52 Hz, 3H) 4.76 (s, 1H) 6.35 (d, J=4.90 Hz, 1H) 6.96 (dd, J=7.91, 4.52 Hz, 1H) 7.11 (dd, J=5.09, 3.58 Hz, 1H) 7.19 (d, J=3.39 Hz, 1H) 7.45 (dd, J=7.91, 4.90 Hz, 1H) 7.57 (d, J=3.77 Hz, 1H) 7.62 (dd, J=7.72, 2.07 Hz, 1H) 7.77-7.89 (m, 1H) 8.56 (dd, J=4.52, 1.88 Hz, 1H) 8.65 (dd, J=4.71, 1.70 Hz, 1H) 8.69 (d, J=2.26 Hz, 1H). Mass: 334.0 (M+H).

### Example 99

### 2-azetidin-1-yl-5-pyridin-3-yl-3-thien-2-yl-3H-pyrido[2,3-e][1,4]diazepine

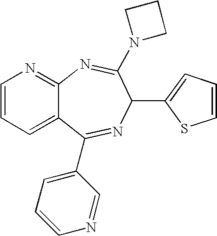

The subtitled compound was synthesized following reaction described above for the example 92f starting with 5-pyridin-3-yl-3-thien-2-yl-1,3-dihydro-2H-pyrido[2,3-e][1,4]diazepin-2-one and azetidine. ¹H NMR (300 MHz, DMSO-D6) δ ppm 0.75 (d, J=6.59 Hz, 1H) 1.96 (d, J=26.00 Hz, 2H) 3.54 (s, 1H) 3.80 (s, 1H) 3.90-4.09 (m, 1H) 4.66 (s, 1H) 6.97 (dd, J=7.72, 4.52 Hz, 1H) 7.08 (dd, J=5.09, 3.58 Hz, 1H) 7.26 (d, J=3.39 Hz, 1H) 7.47 (dd, J=7.82, 4.80 Hz, 1H) 7.54 (d, J=4.90 Hz, 1H) 7.64 (dd, J=7.72, 1.88 Hz, 1H) 7.83-7.92 (m, 1H) 8.57 (dd, J=4.52, 1.88 Hz, 1H) 8.66 (dd, J=4.80, 1.41 Hz, 1H) 8.71 (d, J=1.88 Hz, 1H). Mass 360.0 (M+H).

### Example 100

### N-methyl-5-pyridin-2-yl-3-thien-2-yl-3H-pyrido[2,3-e][1,4]diazepin-2-amine

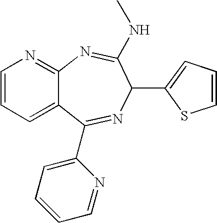

The subtitled compound was synthesized following reaction sequence described above for the example 92 starting with 2-fluoropyridine and 2-pyridine carboxaldehyde. ¹H NMR (300 MHz, DMSO-D6) δ ppm 2.64 (d, J=4.52 Hz, 3H) 4.80 (s, 1H) 6.30 (d, J=4.52 Hz, 1H) 6.92 (dd, J=7.82, 4.62 Hz, 1H) 7.11 (dd, J=4.80, 3.67 Hz, 1H) 7.19 (d, J=3.39 Hz, 1H) 7.46 (dd, J=6.41, 5.09 Hz, 1H) 7.53-7.69 (m, 2H) 7.87-7.98 (m, 1H) 8.00-8.14 (m, 1H) 8.45-8.60 (m, 2H). Mass: 334.1 (M+H).

### Example 101

### N-methyl-3,5-di-2-thienyl-3H-1,4-benzodiazepin-2-amine

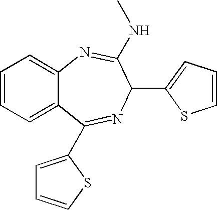

Using an analogous procedure to that described in Example 50 using steps 4 to 8, the titled compound was formed from (2-aminophenyl)(thien-2-yl)methanone. ¹H NMR (300 MHz, CHLOROFORM-d): δ ppm 2.88 (d, J=4.79 Hz, 3H); 4.88 (s, 2H); 7.0-7.6 (m, 9H); 7.71 (d, J=7.86 Hz, 1H). ES (MH⁺)⁺=338

### Example 102

### N-methyl-3,5-di-2-thienyl-3H-pyrido[2,3-e][1,4]diazepin-2-amine

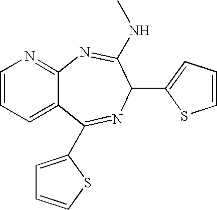

Using analogous procedures to that described in Example 50 and Example 51, the titled compound was formed from thiophene-2-carbaldehyde. ¹H NMR (300 MHz, CHLOROFORM-d): δ ppm 2.94 (d, J=4.58 Hz, 3H); 4.92 (s, 1H); 5.13 (s, br, 1H); 7.0 7.2 (m, 5H); 7.47 (dd, J=13.6, 4.54 Hz, 2H); 8.05 (d, J=7.59 Hz, 1H); 8.71 (d, J=3.70 Hz, 1H). ES (MH⁺)⁺=339

### Example 103

### N-methyl-5-(3-methyl-2-thienyl)-3-(2-thienyl)-3H-pyrido[2,3-e][1,4]diazepin-2-amine

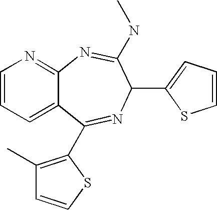

Using analogous procedures to that described in Example 50 and Example 51, the titled compound was formed from 3-methylthiophene-2-carbaldehyde. ¹H NMR (300 MHz, CHLOROFORM-d): δ ppm 2.02 (s, 3H); 2.89 (s, 3H); 4.80 (s, 1H); 5.14 (s, br, 1H); 6.87 (d, 1H); 6.93 (dd, 1H); 7.10 (s, 2H); 7.30 (d, 1H); 7.38 (d, 1H); 7.52 (s, 1H); 7.80 (d, J=7.59 Hz, 1H); 8.62 (d, 1H). ES (MH⁺)⁺=353

### Example 104

### N-methyl-5-(4-methyl-1H-imidazol-5-yl)-3-(2-thienyl)-3H-pyrido[2,3-e][1,4]diazepin-2-amine

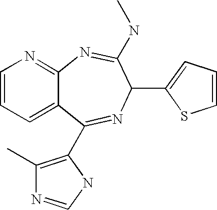

Using analogous procedures to that described in Example 50 and Example 51, the titled compound was formed from 4-methyl-1H-imidazole-5-carbaldehyde. ¹H NMR (300 MHz, DMSO-d₆): δ ppm 2.55 (s, 3H); 2.70 (d, J=4.50 Hz, 3H); 4.72 (s, 1H); 6.18 (d, J=5.2 Hz, 1H); 6.96 (m, 1H); 7.17 (m, 2H); 7.55 (m, 1H); 7.60 (d, J=4.5 Hz, 1H); 7.86 (d, J=7.6 Hz, 1H); 8.50 (d, J=4.5 Hz, 1H); 12.31 (s, 1H). ES (MH⁺)⁺=337

### Example 105

### N-methyl-5-(1,3-thiazol-2-yl)-3-(2-thienyl)-3H-pyrido[2,3-e][1,4]diazepin-2-amine

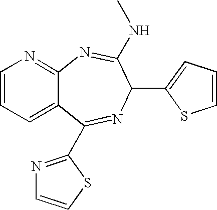

Using analogous procedures to that described in Example 50 and Example 51, the titled compound was formed from 1,3-thiazole-2-carbaldehyde. ¹H NMR (300 MHz, DMSO-d₆): δ ppm 2.75 (d, J=4.6 Hz, 3H); 4.99 (s, 1H); 6.50 (d, J=4.6 Hz, 1H); 7.12 (dd, J=7.9, 4.6 Hz, 1H); 7.22 (dd, J=4.9, 3.6 Hz, 1H); 7.29 (d, J=3.4 Hz, 1H); 7.69 (d, J=4.9 Hz, 1H); 8.00 (d, J=3.2 Hz, 1H); 8.06 (d, J=3.2 Hz, 1H); 8.31 (dd, J=7.7, 1.9 Hz, 1H); 8.65 (dd, J=4.5, 1.9 Hz, 1H). ES (MH⁺)⁺=340.

### Example 106

### 5-(2-furyl)-N-methyl-3-(2-thienyl)-3H-pyrido[2,3-e][1,4]diazepin-2-amine

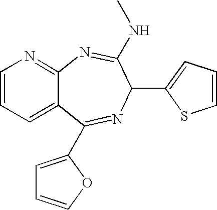

Using analogous procedures to that described in Example 50 and Example 51, the titled compound was formed from furan-2-carbaldehyde. ¹H NMR (400 MHz, DMSO-d₆): δ ppm 2.76 (d, J=4.5 Hz, 3H); 4.86 (s, 1H); 6.40 (d, J=4.5 Hz, 1H); 6.74 (m, 1H); 6.89 (d, J=3.3 Hz, 1H); 7.12 (dd, J=7.8, 4.6 Hz, 1H); 7.22 (m, 1H); 7.28 (d, J=3.1 Hz, 1H); 7.67 (d, J=5 Hz, 1H); 8.00 (s, 1H); 8.03 (dd, J=7.8, 1.7 Hz, 1H); 8.66 (dd, J=4.5, 1.6 Hz), 1H). ES (MH⁺)⁺=323.

### Example 107

### N-methyl-5-(1,3-oxazol-2-yl)-3-thien-2-yl-3H-pyrido[2,3-e][1,4]diazepin-2-amine

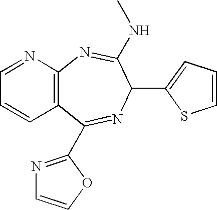

Using analogous procedures to that described in Example 50 and Example 51, the titled compound was formed from 1,3-oxazole-2-carbaldehyde. ¹H NMR (400 MHz, CHLOROFORM-D) δ ppm 2.94 (d, J=4.89 Hz, 3H) 4.96 (s, 1H) 5.15 (d, J=4.16 Hz, 1H) 7.07 (dd, J=7.83, 4.65 Hz, 1H) 7.14-7.21 (m, 1H) 7.24 (d, J=3.42 Hz, 1H) 7.34 (s, 1H) 7.46 (d, J=5.14 Hz, 1H) 7.85 (s, 1H) 8.30 (dd, J=7.83, 1.96 Hz, 1H) 8.72 (dd, J=4.52, 1.83 Hz, 1H); ES (MH⁺)⁺=324; mp=217-219.

### Example 108

### 5-(1H-imidazol-4-yl)-N-methyl-3-thien-2-yl-3H-pyrido[2,3-e][1,4]diazepin-2-amine

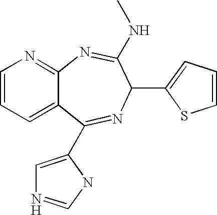

Using analogous procedures to that described in Example 50 and Example 51, the titled compound was formed from 1-{[2-(trimethylsilyl)ethoxy]methyl}-1H-imidazole-4-carbaldehyde. ¹H NMR (400 MHz, DMSO-D6) mixture of tautomers δ ppm 2.75 & 3.22 (d, J=5.38 Hz, 3H) 4.77 & 4.84 (s, 1H) 6.25 & 6.34 (d, J=4.40 Hz, 1H) 7.02-7.14 (m, 3H) 7.18-7.28 (m, 3H) 7.45 & 7.63-7.73 (m, 3H) 7.79 & 7.88 (s, 1H) 8.06 & 8.11 (d, J=7.34 Hz, 1H) 8.58 &) 8.64 (d, J=3.42 Hz, 1H) 12.51 & 12.53 (s, 1H); ES (MH⁺)⁺=323; mp=280-290.

### Example 109

### N-methyl-5-(1-methyl-1H-imidazol-2-yl)-3-thien-2-yl-3H-pyrido[2,3-e][1,4]diazepin-2-amine

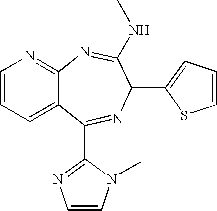

Using analogous procedures to that described in Example 50 and Example 51, the titled compound was formed from 1-methyl-1H-imidazole-2-carbaldehyde. ¹H NMR (300 MHz, CHLOROFORM-D) δ ppm 2.95 (d, J=4.90 Hz, 3H) 4.13 (s, 3H) 4.98 (s, 2H) 6.98-7.10 (m, 2H) 7.12-7.22 (m, 3H) 7.45 (dd, J=5.09, 1.13 Hz, 1H) 8.11 (dd, J=8.01, 1.79 Hz, 1H) 8.70 (dd, J=4.52, 2.07 Hz, 1H); ES (MH⁺)⁺=337; mp=dec>250.

### Example 110

### 2-azetidin-1-yl-5-(1-methyl-1H-imidazol-2-yl)-3-thien-2-yl-3H-pyrido[2,3-e][1,4]diazepine

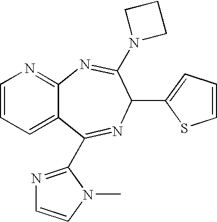

Using analogous procedures to that described in Example 50 and Example 51, the titled compound was formed from 1-methyl-1H-imidazole-2-carbaldehyde except that azetidine was used in Step 8. ¹H NMR (300 MHz, CHLOROFORM-D) δ ppm 2.07 (s, 2H) 3.38 (s, 1H) 3.73 (s, 1H) 4.05 (s, 1H) 4.19 (s, 3H) 4.32 (s, 1H) 4.84 (s, 1H) 6.99 (dd, J=8.10, 4.52 Hz, 1H) 7.08 (s, 1H) 7.13 (d, J=4.71 Hz, 1H) 7.18 (s, 2H) 7.37 (s, 1H) 8.04 (d, J=1.70 Hz, 1H) 8.68 (s, 1H); ES (MH⁺)⁺=363; mp=215-217.

### Example 111

### N-methyl-5-(1-methyl-1H-pyrazol-3-yl)-3-thien-2-yl-3H-pyrido[2,3-e][1,4]diazepin-2-amine

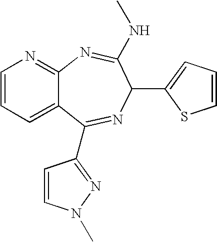

Using analogous procedures to that described in Example 50 and Example 51, the titled compound was formed from 1-methyl-1H-pyrazole-3-carbaldehyde. ¹H NMR (400 MHz, CHLOROFORM-D) δ ppm 2.87-2.96 (d, 3H) 3.92-4.01 (m, 3H) 4.91 (s, 1H) 5.04 (s, 1H) 6.79 (d, J=2.45 Hz, 1H) 6.99 (dd, J=7.70, 4.77 Hz, 1H) 7.12-7.20 (m, 2H) 7.33-7.43 (m, 2H) 8.13 (dd, J=7.70, 2.08 Hz, 1H) 8.66 (dd, J=4.77, 2.08 Hz, 1H); ES (MH⁺)⁺=337.

### Example 112

### N-methyl-5-(1H-pyrrol-2-yl)-3-thien-2-yl-3H-pyrido[2,3-e][1,4]diazepin-2-amine

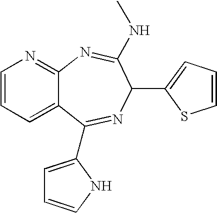

Using analogous procedures to that described in Example 50 and Example 51, the titled compound was formed from 1-{[2-(trimethylsilyl)ethoxy]methyl}-1H-pyrrole-2-carbaldehyde. ¹H NMR (400 MHz, CHLOROFORM-D) δ ppm 2.92 (d, J=4.65 Hz, 3H) 4.94 (s, 1H) 5.02 (d, J=4.16 Hz, 1H) 6.28 (d, J=2.57 Hz, 1H) 6.39 (s, 1H) 6.97-7.05 (m, 2H) 7.10-7.21 (m, 2H) 7.43 (d, J=4.89 Hz, 1H) 8.16 (d, J=7.70 Hz, 1H) 8.68 (d, J=4.65 Hz, 1H) 9.39 (s, 1H); ES (MH⁺)⁺=322; mp=165-170.

### Example 113

### N-methyl-5-(1H-pyrazol-4-yl)-3-thien-2-yl-3H-pyrido[2,3-e][1,4]diazepin-2-amine

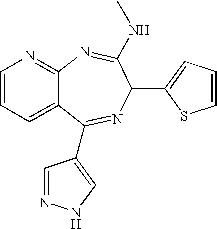

Using analogous procedures to that described in Example 50 and Example 51, the titled compound was formed from 1-{[2-(trimethylsilyl)ethoxy]methyl}-1H-pyrazole-4-carbaldehyde. ¹H NMR (400 MHz, CHLOROFORM-D) δ ppm 2.93 (d, J=4.80 Hz, 3H) 4.89 (s, 1H) 5.11 (d, J=4.80 Hz, 1H) 7.00 (dd, J=7.83, 4.29 Hz, 1H) 7.11-7.21 (m, 2H) 7.44 (d, J=4.29 Hz, 1H) 7.84-7.93 (m, 2H) 7.96 (d, J=7.07 Hz, 1H) 8.70 (d, J=4.55 Hz, 1H) 11.13 (br s, 1H); ES (MH⁺)⁺=323; mp=187-194.

1-{[2-(trimethylsilyl)ethoxy]methyl}-1H-pyrazole-4-carbaldehyde was prepared as follows: To a solution of 4-bromo pyrazole (16.90 g, 115 mmol) in THF (400 mL) under N₂ at 0° C. was added NaH (3.31 g, 138 mmol) and stirred for 15 min. Neat SEM-Cl (21.10 g, 126.46 mmol) was added drop wise to the reaction mixture and stirred overnight at rt. The reaction was quenched with water (200 mL) and excess THF was removed in vacuo. The residue was extracted with EtOAc (2×500 mL), dried over MgSO₄ and removed in vacuo to give crude 4-bromo-1-{[2-(trimethylsilyl)ethoxy]methyl}-1H-pyrazole (36 g) as a yellow oil. The crude material was used in next step without further purification.

To a solution of 4-bromo-1-{[2-(trimethylsilyl)ethoxy]methyl}-1H-pyrazole (35.83 g, 129.35 mmol) in THF (750 mL) at −78° C. under N₂ was added n-BuLi (67.26 mL, 168.16 mmol, 2.5 M) slowly. After the reaction mixture was stirred for 3 h at −78° C., DMF (50 mL) was added at −78° C. and warmed to rt overnight. It was cooled to −30° C., quenched with 1 N HCl (120 mL) and stirred at 0° C. for 30 min and 30 min at rt. The extraction was carried out with EtOAc (2×500 mL) and washed with brine (2×150 mL). The combined organic layers were dried over MgSO₄, filtered and removed in vacuo. The crude material was purified on silica gel and eluted with EtOAc/hexanes (from 5% to 20%) to yield 1-{[2-(trimethylsilyl)ethoxy]methyl}-1H-pyrazole-4-carbaldehyde (12.34 g, 34%) as brown oil.

### Example 114

### N-methyl-5-(1H-pyrazol-3-yl)-3-thien-2-yl-3H-pyrido[2,3-e][1,4]diazepin-2-amine

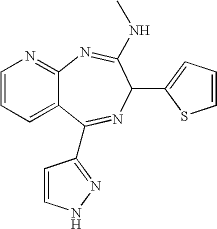

Using analogous procedures to that described in Example 50 and Example 51, the titled compound was formed from 1-{[2-(trimethylsilyl)ethoxy]methyl}-1H-pyrazole-3-carbaldehyde. ¹H NMR (400 MHz, DMSO-D6) mixture of tautomers δ ppm 2.69 & 3.16 (d, J=5.14 Hz, 3H) 4.05-4.14 (m, 1H) 4.78 (s, 1H) 6.25 (d, J=3.91 Hz, 1H) 6.77 (s, 1H) 7.00 (dd, J=7.21, 4.77 Hz, 1H) 7.11-7.18 (m, 1H) 7.21 (s, 1H) 7.61 (d, J=4.65 Hz, 1H) 7.84 (s, 1H) 8.05 (d, J=7.34 Hz, 1H) 8.54 (s, 1H) 13.15 (s, 1H); ES (MH⁺)⁺=323; mp=269-272.

1-{[2-(trimethylsilyl)ethoxy]methyl}-1H-pyrazole-3-carbaldehyde was prepared as follows: 3-Methyl pyrazole (50 g, 0.61 mol) was placed in a 5 L round-bottom flask equipped with mechanical stirrer. 3 L of water was added and heated to 80° C. KMnO₄ (211.90 g, 1.34 mol) was added portion wise and refluxed for 4.5 h. After stirring at rt overnight, solid was filtered and washed with water. The water was removed in vacuo and 100 mL of water was kept in the flask which was acidified with 1 N HCl to pH 4. It was extracted with EtOAc (2×1 L), washed with brine (2×150 mL), dried over MgSO₄, filtered and removed in vacuo to yield 1H-pyrazole-3-carboxylic acid (38 g, 56%) as a white solid.

38 g (0.34 mol) of 1H-pyrazole-3-carboxylic acid was refluxed in anhydrous ethanol (1 L) and conc. sulfuric acid (60 mL) for 20 h under nitrogen. Ethanol was removed and crude was basified to pH 8. Precipitated solid was filtered. The filtrate was extracted with THF/CHCl₃ (2:3, 3×1 L), dried over MgSO₄, filtered and removed in vacuo to yield ethyl 1H-pyrazole-3-carboxylate (39 g, 82%) as a white solid.

To a suspension of ethyl 1H-pyrazole-3-carboxylate (4.42 g, 31.57 mmol) in 1,4-dioxane (140 mL) under N₂ atmosphere at 0° C. was added NaH (0.91 g, 37.88 mmol) and stirred for 15 min. Neat SEM-Cl (5.79 g, 34.73 mmol) was added drop wise to reaction mixture and stirred overnight at rt. It was quenched with water (30 mL) and excess 1,4-dioxane was removed in vacuo. The residue was extracted with EtOAc (2×250 mL), washed with water (1×50 mL), dried over MgSO₄, filtered and removed in vacuo to give crude ethyl 1-{[2-(trimethylsilyl)ethoxy]methyl}-1H-pyrazole-3-carboxylate (8.84 g) as a yellow oil. The crude material was used in next step without purification.

To a suspension of LiAlH₄ in THF (100 mL) at 0° C. under N₂ atmosphere was added a solution of ethyl 1-{[2-(trimethylsilyl)ethoxy]methyl}-1H-pyrazole-3-carboxylate (8.88 g, 32.88 mmol) slowly. After addition was completed the cooling bath was removed and reaction mixture was stirred overnight. It was quenched with water (10 mL) carefully at 0° C. THF was removed and residue was diluted with DCM (250 mL) and organic layer was separated, dried over MgSO₄ and removed in vacuo. The crude material was plugged thru a pad of silica gel with EtOAc/hexanes (from 10% to 100%) to yield (1-{[2-(trimethylsilyl)ethoxy]methyl}-1H-pyrazol-3-yl)methanol (5.80 g, 77%) as an yellow oil. 53.75 g (0.24 mol) of (1-{[2-(trimethylsilyl)ethoxy]methyl}-1H-pyrazol-3-yl)methanol was dissolved in THF and 122.97 g (1.41 mol) of MnO₂ was added. The resulting mixture was refluxed for 60 h. Solid material was filtered through a pad of celite and washed with hot THF. The filtrate was removed in vacuo to give crude product. The crude was plugged thru a pad of silica gel and eluted with EtOAc/hexanes (from 20% to 50%) to yield 1-{[2-(trimethylsilyl)ethoxy]methyl}-1H-pyrazole-3-carbaldehyde (50.88 g, 86.5%) as a red oil.

### Example 115

### 5-(3-furyl)-N-methyl-3-thien-2-yl-3H-pyrido[2,3-e][1,4]diazepin-2-amine

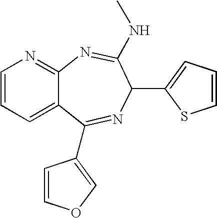

Using analogous procedures to that described in Example 50 and Example 51, the titled compound was formed from 3-furaldehyde except that in step 3 the temperature is 70° C. and time is for 3 h. ¹H NMR (400 MHz, CHLOROFORM-D) δ ppm 2.94 (d, 3H) 4.89 (s, 1H) 5.08 (d, 1H) 6.85 (s, 1H) 6.99 (dd, J=7.83, 4.65 Hz, 1H) 7.11-7.20 (m, 2H) 7.43 (d, J=4.40 Hz, 1H) 7.48 (s, 1H) 7.61 (s, 1H) 7.89-8.18 (m, 1H) 8.68 (d, J=3.67 Hz, 1H); ES (MH⁺)⁺=323; mp=110-114.

### Example 116

### N-methyl-5-(1H-pyrrol-3-yl)-3-thien-2-yl-3H-pyrido[2,3-e][1,4]diazepin-2-amine

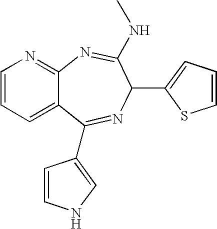

Using analogous procedures to that described in Example 50 and Example 51, the titled compound was formed from 3-furaldehyde except that in step 3 the temperature is 100° C. and time is for 8 h. ¹H NMR (400 MHz, CHLOROFORM-D) δ ppm 2.91 (d, J=4.89 Hz, 3H) 4.88 (s, 1H) 5.04 (d, J=4.65 Hz, 1H) 6.62 (d, J=1.47 Hz, 1H) 6.82 (q, J=2.20 Hz, 1H) 6.95 (dd, J=7.83, 4.65 Hz, 1H) 7.03 (d, J=1.22 Hz, 1H) 7.12-7.17 (m, 2H) 7.41 (dd, J=4.65, 1.22 Hz, 1H) 8.03 (dd, J=7.83, 1.96 Hz, 1H) 8.60 (s, 1H) 8.65 (dd, J=4.65, 1.96 Hz, 1H); ES (MH⁺)⁺=322; mp=187-188 C.

### Example 117

### 2-azetidin-1-yl-5-(1H-pyrrol-3-yl)-3-thien-2-yl-3H-pyrido[2,3-e][1,4]diazepine

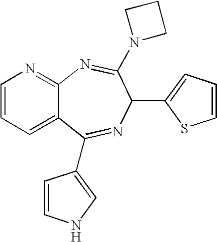

Using an analogous procedure to that described in Example 116, the titled compound was formed except that azetidine was used in the last step. ¹H NMR (300 MHz, CHLOROFORM-D) δ ppm 1.89-2.19 (m, 2H) 3.23-3.48 (m, J=6.78 Hz, 1H) 3.79 (d, J=6.59 Hz, 1H) 4.01 (d, J=6.97 Hz, 1H) 4.19-4.42 (m, J=6.78 Hz, 1H) 4.76 (d, J=0.94 Hz, 1H) 6.62-6.69 (m, 1H) 6.79-6.87 (m, 1H) 6.91 (dd, J=7.82, 4.62 Hz, 1H) 7.00-7.12 (m, 2H) 7.18 (d, J=3.39 Hz, 1H) 7.33 (dd, J=5.09, 1.13 Hz, 1H) 8.01 (dd, J=7.82, 1.98 Hz, 1H) 8.62 (dd, J=4.62, 1.98 Hz, 1H) 8.80 (s, 1H); ES (MH⁺)⁺=348; mp=168-172.

### Example 118

### N-cyclopropyl-5-(1H-pyrrol-3-yl)-3-thien-2-yl-3H-pyrido[2,3-e][1,4]diazepin-2-amine

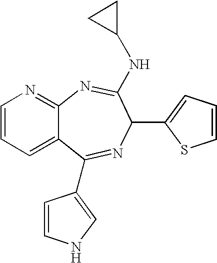

Using an analogous procedure to that described in Example 116, the titled compound was formed except that cyclopropylamine was used in the last step. ¹H NMR (300 MHz, CHLOROFORM-D) δ ppm 0.30-0.43 (m, 1H) 0.43-0.55 (m, 1H) 0.65-0.80 (m, 2H) 2.88 (dd, J=7.06, 3.67 Hz, 1H) 4.83 (s, 1H) 5.08 (d, J=1.88 Hz, 1H) 6.54-6.68 (m, 1H) 6.79-6.87 (m, 1H) 6.96 (dd, J=7.82, 4.62 Hz, 1H) 7.04 (d, J=1.32 Hz, 1H) 7.09-7.16 (m, 2H) 7.40 (dd, J=3.86, 2.35 Hz, 1H) 8.03 (dd, J=7.72, 1.88 Hz, 1H) 8.54 (s, 1H) 8.67 (dd, J=4.62, 1.98 Hz, 1H).

### Example 119

### N-methyl-5-(1-methyl-1H-pyrazol-4-yl)-3-thien-2-yl-3H-pyrido[2,3-e][1,4]diazepin-2-amine

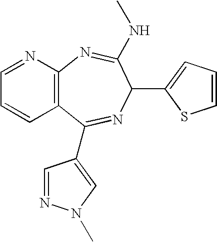

Using analogous procedures to that described in Example 50 and Example 51, the titled compound was formed from 1-methyl-1H-pyrazole-4-carbaldehyde. ¹H NMR (400 MHz, CHLOROFORM-D) δ ppm 2.88-2.95 (d, 3H) 3.90-3.98 (m, 3H) 4.81-4.89 (m, 1H) 5.07 (d, J=4.16 Hz, 1H) 6.99 (dd, J=7.70, 4.28 Hz, 1H) 7.14 (ddd, J=5.75, 3.06, 2.93 Hz, 2H) 7.35-7.45 (m, 1H) 7.71 (s, 2H) 7.96 (d, J=7.83 Hz, 1H) 8.68 (d, J=4.16 Hz, 1H); ES (MH⁺)⁺=337.

### Example 120

### N-methyl-5-(1-methyl-1H-pyrrol-3-yl)-3-thien-2-yl-3H-pyrido[2,3-e][1,4]diazepin-2-amine

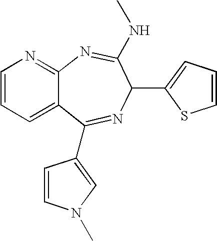

Using analogous procedures to that described in Example 50 and Example 51, the titled compound was formed from 1-methyl-1H-pyrrole-3-carbaldehyde. ¹H NMR (400 MHz, CHLOROFORM-D) δ ppm 2.91 (d, J=4.65 Hz, 3H) 3.64-3.69 (m, 3H) 4.86 (s, 1H) 5.02 (d, J=4.16 Hz, 1H) 6.45 (s, 1H) 6.60 (s, 1H) 6.91 (s, 1H) 6.95 (dd, J=7.83, 4.40 Hz, 1H) 7.11-7.19 (m, 2H) 7.41 (d, J=3.67 Hz, 1H) 8.03 (d, J=7.58 Hz, 1H) 8.64 (d, J=4.65 Hz, 1H); ES (MH⁺)⁺=336.

### Example 121

### N-methyl-5-(1-methyl-1H-pyrrol-2-yl)-3-thien-2-yl-3H-pyrido[2,3-e][1,4]diazepin-2-amine

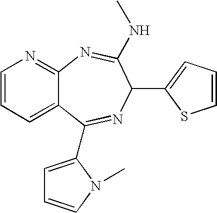

Using analogous procedures to that described in Example 50 and Example 51, the titled compound was formed from 1-methyl-1H-pyrrole-2-carbaldehyde. ¹H NMR (400 MHz, CHLOROFORM-D) δ ppm 2.92 (d, J=4.89 Hz, 3H) 3.98-4.06 (m, 3H) 4.90 (s, 1H) 4.98 (d, J=4.40 Hz, 1H) 6.08-6.17 (m, 2H) 6.81 (s, 1H) 6.94 (dd, J=7.58, 4.65 Hz, 1H) 7.10 (d, J=3.67 Hz, 1H) 7.13-7.17 (m, 1H) 7.41 (d, J=4.89 Hz, 1H) 7.98 (dd, J=7.58, 1.96 Hz, 1H) 8.66 (dd, J=4.65, 1.71 Hz, 1H); ES (MH⁺)⁺=336.

### Example 122

### 5-isothiazol-5-yl-N-methyl-3-thien-2-yl-3H-pyrido[2,3-e][1,4]diazepin-2-amine

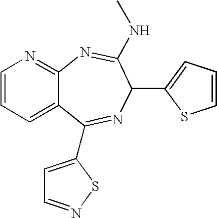

Using analogous procedures to that described in Example 50 and Example 51, the titled compound was formed from isothiazole-5-carbaldehyde. ¹H NMR (400 MHz, CHLOROFORM-D) δ ppm 2.87-3.03 (m, 3H) 4.93 (s, 1H) 5.16 (d, J=3.18 Hz, 1H) 7.04 (dd, J=7.70, 4.77 Hz, 1H) 7.11-7.23 (m, 2H) 7.23-7.33 (m, 2H) 7.46 (d, J=4.89 Hz, 1H) 7.98 (dd, 1H) 8.45-8.57 (m, 1H) 8.74 (d, J=3.18 Hz, 1H); ES (MH⁺)⁺=340; mp=106-109.

### Example 123

### N-methyl-3-thien-2-yl-5-thien-3-yl-3H-pyrido[2,3-e][1,4]diazepin-2-amine

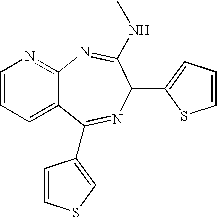

Using analogous procedures to that described in Example 50 and Example 51, the titled compound was formed from thiophene-3-carbaldehyde. ¹H NMR (400 MHz, CHLOROFORM-D) δ ppm 2.87-2.96 (m, 3H) 4.90 (s, 1H) 5.08 (d, J=4.16 Hz, 1H) 6.98 (dd, J=7.58, 4.65 Hz, 1H) 7.12-7.20 (m, 2H) 7.36 (dd, J=4.89, 2.93 Hz, 1H) 7.44 (dd, J=4.52, 1.59 Hz, 1H) 7.51 (d, J=4.89 Hz, 1H) 7.54 (d, J=2.69 Hz, 1H) 7.85-7.93 (m, 1H) 8.69 (dd, J=4.65, 1.96 Hz, 1H); ES (MH⁺)⁺=339; mp=85-88.

### Example 124

### 2-azetidin-1-yl-3-thien-2-yl-5-thien-3-yl-3H-pyrido[2,3-e][1,4]diazepine

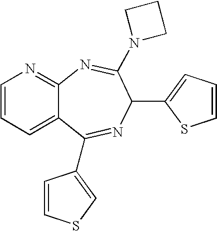

Using an analogous procedure to that described in Example 123, the titled compound was formed except that azetidine was used in the last step. ¹H NMR (300 MHz, CHLOROFORM-D) δ ppm 2.05 (m, 2H) 3.23-3.48 (m, 1H) 3.79 (m, 1H) 4.01 (m, 1H) 4.19-4.42 (m, 1H) 4.76 (d, J=0.94 Hz, 1H) 6.93 (dd, J=7.83, 4.65 Hz, 1H) 7.09 (dd, J=5.01, 3.55 Hz, 1H) 7.20 (d, J=3.18 Hz, 1H) 7.37 (m, 2H) 7.54 (m, 2H) 7.87 (dd, J=7.83, 1.96 Hz, 1H) 8.66 (dd, J=4.40, 1.96 Hz, 1H); ES (MH⁺)⁺=365; mp=90-95.

### Example 125

### N-cyclopropyl-3-thien-2-yl-5-thien-3-yl-3H-pyrido[2,3-e][1,4]diazepin-2-amine

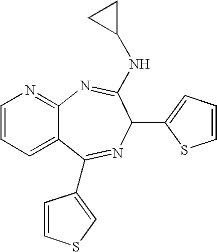

Using an analogous procedure to that described in Example 123, the titled compound was formed except that cyclopropylamine was used in the last step. ¹H NMR (400 MHz, CHLOROFORM-D) δ ppm 0.36 (dt, J=9.60, 4.86 Hz, 1H) 0.42-0.53 (m, J=5.38, 5.01, 4.83, 4.83 Hz, 1H) 0.68-0.80 (m, 2H) 2.90 (td, J=7.34, 3.67 Hz, 1H) 4.80-4.90 (m, 1H) 5.11 (s, 1H) 6.99 (dd, J=7.83, 4.40 Hz, 1H) 7.10-7.20 (m, 2H) 7.32-7.38 (m, 1H) 7.39-7.45 (m, 1H) 7.49 (d, J=4.65 Hz, 1H) 7.55 (d, J=2.20 Hz, 1H) 7.90 (d, J=7.83 Hz, 1H) 8.70 (d, J=4.65 Hz, 1H); ES (MH⁺)⁺=365; mp=90-96.

### Example 126

### N-methyl-3-(5-methylthien-2-yl)-5-thien-2-yl-3H-pyrido[2,3-e][1,4]diazepin-2-amine

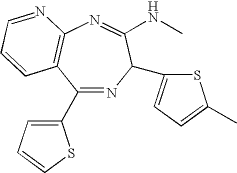

Using analogous procedures to that described in Example 50 (steps 4-6), the titled compound was formed from (2-aminopyridin-3-yl)(2-thienyl)methanone and [(tert-butoxycarbonyl)amino](5-methylthien-2-yl)acetic acid. ES M+H⁺=353. ¹H NMR (300 MHz, CHLOROFORM-D) δ ppm 2.52 (s, 3H) 2.91 (d, J=6 Hz, 3H) 4.80 (s, 1H) 5.21 (d br, J=6 Hz, 1H) 6.76 (d, J=3 Hz, 1H) 6.96-6.98 (m, 2H) 7.05 (m, 1H) 7.12 (d, J=3 Hz, 1H) 7.45 (d, J=6 Hz, 1H) 8.00 (d, J=6 Hz, 1H) 8.66 (d, J=6 Hz, 1H). [(tert-butoxycarbonyl)amino](5-methylthien-2-yl)acetic acid was prepared as follows: amino(5-methylthien-2-yl)acetonitrile, (5 g, 32.7 mmol) was dissolved in 60 mL anhydrous methanol. The solution was cooled in an ice bath and saturated with HCl. The solution was stirred for 2 days at room temperature, then volatiles removed under reduced pressure. The residue was taken up in 200 mL 0.2M HCl and 200 mL ethyl ether. Some dark insoluble material was removed by filtration, and the aqueous layer washed with 100 mL ether. The solution was neutralized with solid NaHCO₃, volatiles removed under reduced pressure and the residue dissolved in 100 mL acetone. Lithium hydroxide (2.1 g, 50 mmol) dissolved in 10 mL water was added and the solution was stirred overnight. Volatiles were removed under reduced pressure and the residue dissolved in 200 mL water and brought to pH 12 with NaOH. The solution was washed three times with 75 mL portions of ethyl ether and concentrated at reduced pressure to approximately 75 mL. Boc anhydride (10 g, 45.8 mmol) was dissolved in 75 mL dioxane and added to the amino acid solution. The mixture was stirred for 2 days at room temperature, dioxane was removed at reduced pressure and the resulting solution washed three times with 50 mL portions of ethyl ether. The aqueous layer was cooled in an ice bath and brought to pH 4 with 1N HCl. The solution was washed four times with 100 mL portions of dichloromethane. The combined organic fractions were dried over MgSO₄, and volatiles were removed under reduced pressure to give 5.2 g (50%) of a light brown oily solid sufficiently pure to be used in subsequent steps. ¹H NMR (300 MHz, CHLOROFORM-D) δ ppm 1.3-1.5 (m br, 9H) 2.43 (s, 3H) 5.48 (s, 1H) 6.58 (d, J=3 Hz, 1H) 6.86 (s, J=3 Hz, 1H)

### Example 127

### 3-(5-chlorothien-2-yl)-N-methyl-5-thien-2-yl-3H-pyrido[2,3-e][1,4]diazepin-2-amine

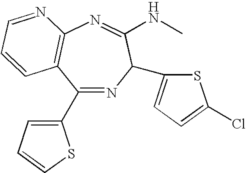

Amino(5-chlorothien-2-yl)acetic acid was Boc-protected in aqueous dioxane following a procedure analogous to that described in Example 126. The perfluorophenyl ester was prepared by treating a mixture of [(tert-butoxycarbonyl)amino](5-chlorothien-2-yl)acetic acid (1 eq) and pentafluorophenol (1.1 eq.) in dichloromethane with DCC (1.2 eq.) for one hour. DCC urea was removed by filtration and the crude product was purified by chromatography (3% ethyl acetate in dichloromethane).

3-(5-chlorothien-2-yl)-5-thien-2-yl-1,3-dihydro-2H-pyrido[2,3-e][1,4]diazepin-2-one: (2-Aminopyridin-3-yl)(2-thienyl)methanone (0.700 g, 3.43 mmol) and pentafluorophenyl [(tert-butoxycarbonyl)amino](5-chlorothien-2-yl)acetate (1.57 g, 3.43 mmol) were dissolved in 10 mL anhydrous 1,2 dichloroethane under nitrogen. The solution was heated for 45 minutes at 130° C. in a microwave reactor. Volatiles were removed under reduced pressure and the product purified by chromatography on silica (5% methanol in dichloromethane. The Boc group was removed by heating the product in 20 mL 30% trifluoroacetic acid/dichloromethane at 40° C. for one hour. Volatiles were removed under reduced pressure and the residue dissolved in 10 mL glacial acetic acid. Ammonium acetate (2 g) was added and the mixture stirred at room temperature for 48 hours. Volatiles were removed under reduced pressure and the residue partitioned in 300 mL dichloromethane/100 mL saturated NaHCO₃ solution. A precipitate was filtered off. The organic layer was evaporated under reduced pressure and the residue suspended in 20 mL methanol. The suspension was filtered and the resulting solid combined with the previously isolated precipitate to give 650 mg §0 (53%) of 3-(5-chlorothien-2-yl)-5-thien-2-yl-1,3-dihydro-2H-pyrido[2,3-e][1,4]diazepin-2-one as an off white solid. The amide was converted to the amidine following the procedure described in Example 51 (step 7) ES M+H⁺=373. ¹H NMR (300 MHz, DMSO-D6) δ ppm 2.69 (d, J=4.52 Hz, 3H) 4.78 (s, 1H) 6.69 (d, J=4.52 Hz, 1H) 7.05 (m, 2H) 7.15 (m, 3H) 7.77 (dd, J=4.90, 1.13 Hz, 1H) 8.02 (dd, J=7.82, 1.98 Hz, 1H) 8.60 (dd, J=4.62, 1.98 Hz, 1H)

### Example 128

### 3-(5-chlorothien-2-yl)-N-cyclopropyl-5-thien-2-yl-3H-pyrido[2,3-e][1,4]diazepin-2-amine

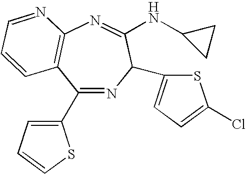

Using an analogous procedure to that described in Example 127, the titled compound was prepared. ES M+H⁺=399. ¹H NMR (300 MHz, CHLOROFORM-D) δ ppm 0.40 (m, 1H) 0.53 (m, 1H) 0.80 (m, 2H) 2.92 (m, 1H) 4.76 (m, 1H) 5.17 (br. s, 1H) 6.87 (dd, J=3.77, 0.94 Hz, 1H) 6.93 (m, 1H) 7.05 (m, 2H) 7.17 (dd, J=3.77, 0.94 Hz, 1H) 7.49 (dd, J=4.99, 1.04 Hz, 1H) 8.03 (dd, J=7.82, 1.98 Hz, 1H) 8.71 (dd, J=4.71, 1.88 Hz, 1H)

### Example 129

### N-methyl-3-(3-methylthien-2-yl)-5-thien-2-yl-3H-pyrido[2,3-e][1,4]diazepin-2-amine

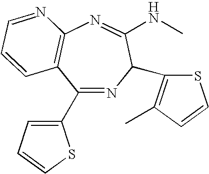

Using analogous procedures to those described in Example 127, the titled compound was formed from (2-Aminopyridin-3-yl)(2-thienyl)methanone and pentafluorophenyl [(tert-butoxycarbonyl)amino](3-methylthien-2-yl)acetate. ES M+H⁺=353. ¹H NMR (300 MHz, CHLOROFORM-D) δ ppm 2.03 (s, 3H) 2.87 (d, J=4.90 Hz, 3H) 4.71 (s, 1H) 5.02 (d, J=4.52 Hz, 1H) 6.88 (d, J=5.09 Hz, 1H) 6.94 (dd, J=7.82, 4.62 Hz, 1H) 7.01 (dd, J=4.99, 3.67 Hz, 1H) 7.08 (dd, J=3.77, 1.13 Hz, 1H) 7.30 (d, J=5.09 Hz, 1H) 7.41 (dd, J=4.99, 1.04 Hz, 1H) 7.97 (dd, J=7.82, 1.98 Hz, 1H) 8.63 (dd, J=4.62, 1.98 Hz, 1H)

### Example 130

### N-cyclopropyl-3-(3-methylthien-2-yl)-5-thien-2-yl-3H-pyrido[2,3-e][1,4]diazepin-2-amine

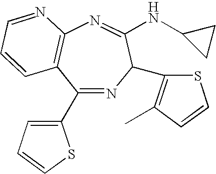

Using an analogous procedure to that described in Example 129, the titled compound was formed. ES M+H⁺=379. ¹H NMR (300 MHz, CHLOROFORM-D) δ ppm 0.35 (m, 1H) 0.48 (m, 1H) 0.75 (m, 2H) 2.07 (s, 3H) 2.88 (m, 1H) 4.71 (s, 1H) 5.08 (d, J=2.26 Hz, 1H) 6.92 (d, J=5.09 Hz, 1H) 7.00 (dd, J=7.82, 4.62 Hz, 1H) 7.07 (dd, J=5.09, 3.77 Hz, 1H) 7.15 (dd, J=3.77, 1.13 Hz, 1H) 7.34 (d, J=5.09 Hz, 1H) 7.47 (dd, J=5.09, 1.13 Hz, 1H) 8.02 (dd, J=7.82, 1.98 Hz, 1H) 8.71 (dd, J=4.62, 1.98 Hz, 1H)

### Example 131

### 5-(2-furyl)-N-methyl-3-(3-methylthien-2-yl)-3H-pyrido[2,3-e][1,4]diazepin-2-amine

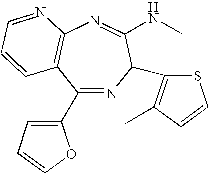

Using analogous procedures to that described in Example 6 (steps 2-3), the titled compound was formed from methyl amino(3-methylthien-2-yl)acetate hydrochloride and 3-[imino(thien-2-yl)methyl]pyridin-2-amine ES M+H⁺=337. ¹H NMR (300 MHz, CHLOROFORM-D) δ ppm 2.12 (m, 3H) 2.92 (d, J=4.90 Hz, 3H) 4.77 (s, 1H) 5.09 (s, 1H) 6.50 (dd, J=3.49, 1.79 Hz, 1H) 6.77 (d, J=3.39 Hz, 1H) 6.98 (m, 2H) 7.35 (d, J=4.90 Hz, 1H) 7.61 (s, 1H) 7.98 (dd, J=7.82, 1.98 Hz, 1H) 8.69 (dd, J=4.62, 1.98 Hz, 1H)

### Example 132

### N-methyl-3-(1,3-thiazol-2-yl)-5-thien-2-yl-3H-pyrido[2,3-e][1,4]diazepin-2-amine

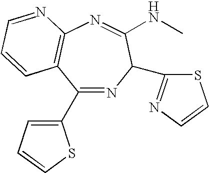

Using analogous procedures to that described in Example 6 (steps 2-3), the titled compound was formed from methyl amino(1,3-thiazol-2-yl)acetate hydrochloride and 3-[imino(thien-2-yl)methyl]pyridin-2-amine. ES M+H⁺=340. ¹H NMR (300 MHz, DMSO-D6) δ ppm 2.85 (d, J=3.58 Hz, 3H) 5.46 (s, 1H) 7.22 (dd, J=4.90, 3.96 Hz, 1H) 7.31 (d, J=3.58 Hz, 1H) 7.43 (dd, J=7.82, 5.93 Hz, 1H) 7.91 (m, 2H) 8.01 (d, J=3.20 Hz, 1H) 8.19 (d, J=4.52 Hz, 1H) 8.64 (dd, J=7.82, 1.41 Hz, 1H) 8.70 (dd, J=5.65, 1.51 Hz, 1H)

### Example 133

### 5-(2-furyl)-N-methyl-3-(1,3-thiazol-2-yl)-3H-pyrido[2,3-e][1,4]diazepin-2-amine

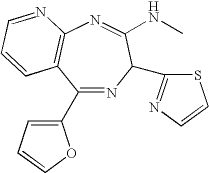

Using analogous procedures to that described in Example 6 (steps 2-3), the titled compound was formed from methyl amino(1,3-thiazol-2-yl)acetate hydrochloride and 3-[2-furyl(imino)methyl]pyridin-2-amine. ES M+H⁺=324. ¹H NMR (300 MHz, CHLOROFORM-D) δ ppm 2.96 (d, J=4.90 Hz, 3H) 4.88 (s, 1H) 5.89 (s, 1H) 6.52 (dd, J=3.30, 1.79 Hz, 1H) 6.78 (d, J=3.20 Hz, 1H) 7.00 (dd, J=7.82, 4.62 Hz, 1H) 7.48 (d, J=3.20 Hz, 1H) 7.61 (d, J=0.75 Hz, 1H) 7.94 (d, J=3.20 Hz, 1H) 8.01 (dd, J=7.72, 1.88 Hz, 1H) 8.69 (dd, J=4.52, 1.88 Hz, 1H)

### Example 134

### 3-cyclopentyl-5-(2-furyl)-N-methyl-3H-pyrido[2,3-e][1,4]diazepin-2-amine

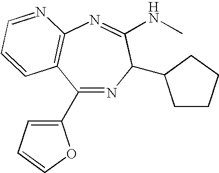

Using analogous procedures to that described in Example 6 (steps 2-3), the titled compound was formed from methyl amino(cyclopentyl)acetate hydrochloride and 3-[2-furyl(imino)methyl]pyridin-2-amine. ES M+H⁺=309. ¹H NMR (300 MHz, CHLOROFORM-D) δ ppm 1.36 (m, 3H) 1.59 (m, 4H) 1.95 (m, 1H) 2.20 (dd, J=12.15, 5.56 Hz, 1H) 2.81 (m, 1H) 3.01 (d, J=4.71 Hz, 3H) 5.35 (d, J=3.77 Hz, 1H) 6.49 (dd, J=3.39, 1.70 Hz, 1H) 6.62 (d, J=3.39 Hz, 1H) 6.98 (dd, J=7.82, 4.62 Hz, 1H) 7.58 (d, J=0.94 Hz, 1H) 7.99 (dd, J=7.82, 1.98 Hz, 1H) 8.66 (dd, J=4.62, 1.98 Hz, 1H)

### Example 135

### 3-(4-chloro-2-thienyl)-N-methyl-5-(2-thienyl)-3H-pyrido[2,3-e][1,4]diazepin-2-amine

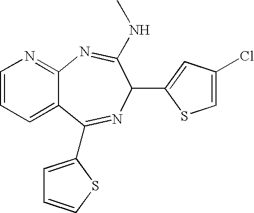

Using analogous procedures to that described in Example 50 and Example 51, the titled compound was formed from thiophene-3-carbaldehyde and [(tert-butoxycarbonyl)amino](4-chlorothien-2-yl)acetic acid. ¹H NMR (300 MHz, CHLOROFORM-d): δ ppm 2.98 (s, 3H); 4.85 (s, 1H); 5.14 (s, br, 1H); 7.0-7.4 (m, 5H); 7.52 (s, 1H); 8.09 (s, 1H); 8.73 (s, 1H). ES (MH⁺)⁺=373

### Example 136

### 3-(3-chloro-2-thienyl)-N-methyl-5-(2-thienyl)-3H-pyrido[2,3-e][1,4]diazepin-2-amine

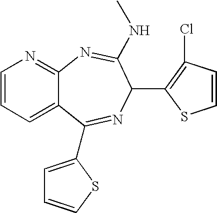

Using analogous procedures to that described in Example 50 and Example 51, the titled compound was formed from thiophene-3-carbaldehyde and [(tert-butoxycarbonyl)amino](3-chlorothien-2-yl)acetic acid. ¹H NMR (300 MHz, CHLOROFORM-d): δ ppm 2.92 (d, J=5.20 Hz, 3H); 4.79 (s, 1H); 5.18 (s, br, 1H); 6.98-7.08 (m, 4H); 7.44 (dd, J=8.0 Hz, 5.30 Hz, 2H); 8.05 (d, J=7.50 Hz, 1H); 8.68 (d, J=4.6 Hz, 1H). ES (MH⁺)⁺=373

Utility

The compounds of the present invention have utility for the prevention and treatment of H. pylori infection. Methods of treatment target the prevention of peptidoglycan biosynthesis through the MurI enzyme. Compounds that inhibit MurI activity control the production of cell wall biosynthesis. The inhibition of MurI will inhibit growth of H. pylori and will reduce or prevent the diseases resulting from H. pylori infection such as gastritis, mucosa-associated lymphoid tissue lymphoma, gastric adenocarcinoma, adenocarcinoma of the stomach, or ulcer diseases including but not limited to pepetic ulcer. The compounds of the present invention have utility for the prevention and treatment of such disorders.

Compounds of the present invention have been shown to inhibit MurI, as determined by glutamate racemase activity assay described herein.

Compounds provided by this invention should also be useful as standards and reagents in determining the ability of a potential pharmaceutical to inhibit MurI. These would be provided in commercial kits comprising a compound of this invention.

Abbreviations

As used herein “rt” denotes room temperature, “ug” denotes microgram, “mg” denotes milligram, “g” denotes gram, “uL” denotes microliter, “mL” denotes milliliter, “L” denotes liter, “nM” denotes nanomolar, “uM” denotes micromolar, “mM” denotes millimolar, “M” denotes molar, “nm” denotes nanometer, “DMSO” denotes dimethyl sulfoxide, “DTT” denotes dithiothreitol, “EDTA” denotes ethylenediaminetetraacetate,

Assay

Glutamate Racemase Activity Assay:

Glutamate racemase (MurI) activity was assayed by measuring the conversion of glutamate from D to L enantiomer. This reaction was coupled to the reduction of NAD⁺ to NADH by L-glutamate dehydrogenase (LGDH). LGDH from bovine liver was obtained as a lyophilized powder (Roche #197734) and dissolved in buffer containing 10 mM Tris (Sigma #T-6791), pH 7.5, 0.1 mM EDTA (Fisher #BP118-500) and 50% (weight/volume) glycerol (Sigma #G-9012). The assay mixture consisted of 100 mM Tris-HCl, pH 8.0, 10 mM β-NAD (Sigma #N-1511), 5 mM DTT (Sigma #D-5545), 0.03% PEG (mw 8000, Sigma #P-5413), 0.03 mg/mL BSA (Pierce #23210), 15 U/mL LGDH, D-glutamate (40 μM, Fluka #49460), and purified MurI (50 nM or 1 uM). The assay was performed in 96-well black microtiter plates (Greiner #XN²-9511) in a final assay volume of 100 μL. Compounds were prepared as 20 mM stock solutions in dimethyl sulfoxide (DMSO, Sigma #D-5879) and serial dilutions were prepared from these solutions using DMSO, 2 μL of which were added to the wells. Activity at room temperature was measured by monitoring the increase in fluorescence using a TECAN Ultra plate reader with 340 nm excitation and 465 nm emission filters. The compounds described have a measured IC₅₀ in this assay of less than 400 μM

1. A compound having the structural formula (I): 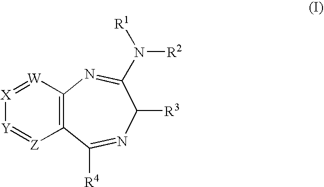
wherein:
W is N or CR^{a};
X is N or CR^{a};
Y is N or CR^{a};
Z is N or CR^{a};
provided however, only one of W, X, Y or Z is N;
R¹ and R² are, at each occurrence independently selected from H, or optionally substituted C₁₋₅alkyl, optionally substituted C₂₋₅alkenyl, optionally substituted C₂₋₅alkynyl, optionally substituted C₃₋₇cycloalkyl, optionally substituted C₃₋₇cycloalkyl-C₁₋₃alkyl, optionally substituted N—C₁₋₅alkyl, optionally substituted N—C₃₋₇cycloalkyl, optionally substituted O—C₁₋₅alkyl, optionally substituted heterocyclyl, or optionally substituted phenyl; or R¹ and R² and the N to which they are attached in combination form an optionally substituted 3-7 member saturated, unsaturated or aromatic ring having 1, 2, 3 or 4 nitrogen atoms, and 0 or 1 oxygen atoms, and 0 or 1 sulfur atoms;
R³ is selected from optionally substituted C₃₋₇cycloalkyl, optionally substituted C₅₋₇cycloalkenyl, optionally substituted heterocyclyl, or optionally substituted phenyl;
R⁴ is selected from optionally substituted C₃₋₇cycloalkyl, optionally substituted C₅₋₇cycloalkenyl, optionally substituted heterocyclyl, or optionally substituted phenyl;
R^{a} is independently selected from H, cyano, halogen, nitro, C₁₋₅alkyl, C₂₋₅alkenyl, C₂₋₅ alkynyl, hydroxyl, amino, C₁₋₅alkylamino, di(C₁₋₅ alkyl)amino, C₁₋₅alkoxyl, C₁₋₅alkylthio each substituted by zero, one or more of halogen, cyano, amino, hydroxyl, oxo carboxylate, CO₂—C₁₋₆alkyl, CONH₂, CONH—C₁₋₆alkyl, CON(C₁₋₆alkyl)-C₁₋₆alkyl, SO(C₁₋₆alkyl), SO₂(C₁₋₆alkyl), SO₂NH—C₁₋₆alkyl, SO₂NH₂, and SO₂N(C₁₋₆alkyl)-C₁₋₆alkyl.

2. A compound as recited in claim 1 wherein:
R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl or optionally substituted O—C₁₋₅alkyl, or R¹ and R² and the N to which they are attached in combination form an optionally substituted 4-5 member saturated, unsaturated or aromatic ring having 1 additional nitrogen atoms.

3. A compound as recited in claim 1 wherein:
R³ is selected from optionally substituted heterocyclyl, or optionally substituted phenyl.

4. A compound as recited in claim 1 wherein:
R⁴ is selected from optionally substituted heterocyclyl, or optionally substituted phenyl, or optionally substituted C₃₋₇cycloalkyl.

5. A compound as recited in claim 1 wherein:
R^{a} is independently selected from H, NH₂, OH, halogen, C₁₋₅alkyl, cyano, nitro, trifluoromethyl, alkoxy, alkylamino.

6. A compound as recited in claim 1 wherein:
W is N or CR^{a};
X is N or CR^{a};
Y is N or CR^{a};
Z is N or CR^{a};
provided however, only one of W, X, Y or Z is N;
R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl or optionally substituted O—C₁₋₅alkyl, or R¹ and R² and the N to which they are attached in combination form an optionally substituted 4-5 member saturated, unsaturated or aromatic ring having 1 additional nitrogen atoms;
R³ is selected from optionally substituted heterocyclyl, optionally substituted phenyl;
R⁴ is selected from optionally substituted heterocyclyl, or optionally substituted phenyl or optionally substituted C₃₋₇cycloalkyl;
R^{a} is independently selected from H, NH₂, OH, halogen, C₁₋₅alkyl, cyano, nitro, trifluoromethyl, alkoxy, alkylamino.

7. A compound as recited in claim 1 wherein:
W is N;
X is CR^{a};
Y is CR^{a};
Z is CR^{a};
R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl or optionally substituted O—C₁₋₅alkyl, or R¹ and R² and the N to which they are attached in combination form an optionally substituted 4-5 member saturated, unsaturated or aromatic ring having 1 additional nitrogen atoms;
R³ is selected from optionally substituted heterocyclyl, optionally substituted phenyl;
R⁴ is selected from optionally substituted heterocyclyl, or optionally substituted phenyl or optionally substituted C₃₋₇cycloalkyl;
R^{a} is independently selected from H, NH₂, OH, halogen, C₁₋₅alkyl, cyano, nitro, trifluoromethyl, alkoxy, alkylamino.

8. A compound as recited in claim 1 wherein:
W is N or CR^{a};
X is N or CR^{a};
Y is N or CR^{a};
Z is N or CR^{a};
provided however, only one of W, X, Y or Z is N;
R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl, or optionally substituted O—C₁₋₅alkyl;
R³ is selected from optionally substituted heterocyclyl, optionally substituted phenyl;
R⁴ is selected from optionally substituted heterocyclyl, or optionally substituted phenyl or optionally substituted C₃₋₇cycloalkyl;
R^{a} is independently selected from H, NH₂, OH, halogen, C₁₋₅alkyl, cyano, nitro, trifluoromethyl, alkoxy, alkylamino.

9. A compound as recited in claim 1 wherein:
W is N;
X is CR^{a};
Y is CR^{a};
Z is CR^{a};
R¹ and R² are, at each occurrence, independently selected from H, optionally substituted C₁₋₅alkyl, optionally substituted C₂₋₅alkenyl, optionally substituted C₂₋₅alkynyl, optionally substituted C₃₋₇cycloalkyl, optionally substituted N—C₁₋₅alkyl, optionally substituted N—C₄₋₆cycloalkyl, or optionally substituted O—C₁₋₅alkyl;
R³ is selected from optionally substituted heterocyclyl, optionally substituted phenyl;
R⁴ is selected from optionally substituted heterocyclyl, or optionally substituted phenyl or optionally substituted C₃₋₇cycloalkyl;
R^{a} is independently selected from H, NH₂, OH, halogen, C₁₋₅alkyl, cyano, nitro, trifluoromethyl, alkoxy, alkylamino.

10. A compound as recited in claim 1 wherein:
W is N or CR^{a};
X is N or CR^{a};
Y is N or CR^{a};
Z is N or CR^{a};
provided however, only one of W, X, Y or Z is N;
R¹ and R² are at each occurrence, independently selected from H, optionally substituted C₁₋₅alkyl, optionally substituted C₃₋₇cycloalkyl, optionally substituted N—C₁₋₅alkyl or optionally substituted N—C₄₋₆cycloalkyl;
R³ is selected from optionally substituted heterocyclyl, optionally substituted phenyl;
R⁴ is selected from optionally substituted heterocyclyl, or optionally substituted phenyl or optionally substituted C₃₋₇cycloalkyl;
R^{a} is independently selected from H, NH₂, OH, halogen, C₁₋₅alkyl.

11. A compound as recited in claim 1 wherein:
W is N;
X is CR^{a};
Y is CR^{a};
Z is CR^{a};
R¹ and R² are at each occurrence, independently selected from H, optionally substituted C₁₋₅alkyl, optionally substituted C₃₋₇cycloalkyl, optionally substituted N—C₁₋₅alkyl or optionally substituted N—C₄₋₆cycloalkyl;
R³ is selected from optionally substituted heterocyclyl, optionally substituted phenyl;
R⁴ is selected from optionally substituted heterocyclyl, or optionally substituted phenyl or optionally substituted C₃₋₇cycloalkyl;
R^{a} is independently selected from H, NH₂, OH, halogen, C₁₋₅alkyl.

12. A compound selected from:
5-(4-fluorophenyl)-N-methyl-3-phenyl-3H-1,4-benzodiazepin-2-amine;
5-(4-fluorophenyl)-N-methyl-3-thien-2-yl-3H-1,4-benzodiazepin-2-amine;
5-(4-fluorophenyl)-3-phenyl-3H-1,4-benzodiazepin-2-amine;
2-azetidin-1-yl-5-(4-fluorophenyl)-3-phenyl-3H-1,4-benzodiazepine;
N-ethyl-5-(4-fluorophenyl)-3-phenyl-3H-1,4-benzodiazepin-2-amine;
N-methyl-5-(4-methylphenyl)-3-thien-2-yl-3H-pyrido[2,3e][1,4]diazepin-2-amine;
5-(4-methoxyphenyl)-N-methyl-3-thien-2-yl-3H-pyrido[2,3-e][1,4]diazepin-2-amine;
N-cyclopropyl-5-(4-methyl phenyl)-3-thien-2-yl-3H-pyrido[2,3-e][1,4]diazepin-2-amine;
N-methyl-3-pyridin-4-yl-5-thien-2-yl-3H-pyrido[2,3-e][1,4]diazepin-2-amine;
N-ethyl-3-pyridin-4-yl-5-thien-2-yl-3H-pyrido[2,3-e][1,4]diazepin-2-amine;
N-cyclopropyl-3-pyridin-4-yl-5-thien-2-yl-3H-pyrido[2,3-e][1,4]diazepin-2-amine;
3-(2-amino-1,3-thiazol-4-yl)-N-methyl-5-thien-2-yl-3H-pyrido[2,3-e][1,4]diazepin-2-amine;
5-(6-aminopyridin-3-yl)-N-methyl-3-thien-2-yl-3H-pyrido[2,3-e][1,4]diazepin-2-amine;
5-(2-aminopyridin-4-yl)-N-methyl-3-thien-2-yl-3H-pyrido[2,3-e][1,4]diazepin-2-amine;
5-(2-aminopyridin-4-yl)-N-cyclopropyl-3-thien-2-yl-3H-pyrido[2,3-e][1,4]diazepin-2-amine;
3-(3-chlorothien-2-yl)-5-(2-furyl)-N-methyl-3H-pyrido[2,3-e][1,4]diazepin-2-amine;
3-(4-bromothien-2-yl)-5-(4-fluorophenyl)-N-methyl-3H-pyrido[2,3-e][1,4]diazepin-2-amine;
3-(4-bromothien-2-yl)-N-methyl-5-thien-2-yl-3H-pyrido[2,3-e][1,4]diazepin-2-amine;
5-[2-(methylamino)-5-thien-2-yl-3H-pyrido[2,3-e][1,4]diazepin-3-yl]thiophene-3-carbonitrile;
3-(4-bromothien-2-yl)-5-(2-furyl)-N-methyl-3H-pyrido[2,3-e][1,4]diazepin-2-amine;
3-(2-furyl)-N-methyl-5-thien-2-yl-3H-pyrido[2,3-e][1,4]diazepin-2-amine;
3,5-di-2-furyl-N-methyl-3H-pyrido[2,3-e][1,4]diazepin-2-amine;
5-(2-furyl)-3-(4-iodothien-2-yl)-N-methyl-3H-pyrido[2,3-e][1,4]diazepin-2-amine;
3-{5-[5-(2-furyl)-2-(methylamino)-3H-pyrido[2,3-e][1,4]diazepin-3-yl]thien-3-yl}prop-2-yn-1-ol;
3-{4-[3-(dimethylamino)prop-1-ynyl]thien-2-yl}-5-(2-furyl)-N-methyl-3H-pyrido[2,3-e][1,4]diazepin-2-amine;
4-{5-[5-(2-furyl)-2-(methylamino)-3H-pyrido[2,3-e][1,4]diazepin-3-yl]thien-3-yl}-2-methylbut-3-yn-2-ol;
4-{5-[5-(2-furyl)-2-(methylamino)-3H-pyrido[2,3-e][1,4]diazepin-3-yl]thien-3-yl}but-3-yn-2-ol;
4-{5-[5-(2-furyl)-2-(methylamino)-3H-pyrido[2,3-e][1,4]diazepin-3-yl]thien-3-yl}but-3-yn-1-ol;
N-(3-{5-[5-(2-furyl)-2-(methylamino)-3H-pyrido[2,3-e][1,4]diazepin-3-yl]thien-3-yl}prop-2-ynyl)acetamide;
5-(4-fluorophenyl)-N,8-dimethyl-3-phenyl-3H-pyrido[2,3-e][1,4]diazepin-2-amine;
N-cyclopropyl-5-(4-fluorophenyl)-8-methyl-3-thien-2-yl-3H-pyrido[2,3-e][1,4]diazepin-2-amine;
N,8-dimethyl-5-phenyl-3-thien-2-yl-3H-pyrido[2,3-e][1,4]diazepin-2-amine;
5-(4-fluorophenyl)-N,8-dimethyl-3-thien-2-yl-3H-pyrido[2,3-e][1,4]diazepin-2-amine;
N²-cyclopropyl-5-(4-fluorophenyl)-3-thien-2-yl-3H-pyrido[2,3-e][1,4]diazepine-2,8-diamine;
5-(4-fluorophenyl)-N²-methyl-3-thien-2-yl-3H-pyrido[2,3-e][1,4]diazepine-2,8-diamine;
7-chloro-N-methyl-3,5-dithien-2-yl-3H-pyrido[2,3-e][1,4]diazepin-2-amine;
N-cyclopropyl-5-(2-furyl)-3-thien-2-yl-3H-pyrido[2,3-e][1,4]diazepin-2-amine;
N-ethyl-5-(2-furyl)-3-thien-2-yl-3H-pyrido[2,3-e][1,4]diazepin-2-amine;
5-(2-furyl)-N-prop-2-ynyl-3-thien-2-yl-3H-pyrido[2,3-e][1,4]diazepin-2-amine;
2-azetidin-1-yl-5-(2-furyl)-3-thien-2-yl-3H-pyrido[2,3-e][1,4]diazepine;
7-fluoro-5-(2-furyl)-N-methyl-3-thien-2-yl-3H-pyrido[2,3-e][1,4]diazepin-2-amine;
N-methyl-3-pyridin-3-yl-5-thien-2-yl-3H-pyrido[2,3-e][1,4]diazepin-2-amine;
5-(2-furyl)-N,8-dimethyl-3-thien-2-yl-3H-pyrido[2,3-e][1,4]diazepin-2-amine;
N,8-dimethyl-3,5-dithien-2-yl-3H-pyrido[2,3-e][1,4]diazepin-2-amine;
2-(2,2-dimethyl hydrazino)-3,5-dithien-2-yl-3H-pyrido[2,3-e][1,4]diazepine;
5-(2-furyl)-N-methyl-3-(1,3-thiazol-5-yl)-3H-pyrido[2,3-e][1,4]diazepin-2-amine;
N-methyl-3-(1,3-thiazol-5-yl)-5-thien-2-yl-3H-pyrido[2,3-e][1,4]diazepin-2-amine;
N-cyclopropyl-5-(2-furyl)-3-(1,3-thiazol-5-yl)-3H-pyrido[2,3-e][1,4]diazepin-2-amine;
N-methyl-3-(1H-pyrazol-4-yl)-5-thien-2-yl-3H-pyrido[2,3-e][1,4]diazepin-2-amine;
N-methyl-3,5-di-2-thienyl-3H-pyrido[2,3-e][1,4]diazepin-2-amine;
5-cyclohexyl-N-methyl-3-thien-2-yl-3H-pyrido[2,3-e][1,4]diazepin-2-amine;
2-azetidin-1-yl-5-cyclohexyl-3-thien-2-yl-3H-pyrido[2,3-e][1,4]diazepine;
5-cyclopentyl-N-methyl-3-thien-2-yl-3H-pyrido[2,3-e][1,4]diazepin-2-amine;
N-methyl-5-phenyl-3-(2-thienyl)-3H-1,4-benzodiazepin-2-amine;
N-methyl-3,5-diphenyl-3H-1,4-benzodiazepin-2-amine;
7-chloro-N-methyl-3,5-diphenyl-3H-1,4-benzodiazepin-2-amine;
N-methyl-5-phenyl-3-thien-3-yl-3H-1,4-benzodiazepin-2-amine;
N-ethyl-5-phenyl-3-thien-2-yl-3H-1,4-benzodiazepin-2-amine;
5-phenyl-N-propyl-3-thien-2-yl-3H-1,4-benzodiazepin-2-amine;
N-isopropyl-5-phenyl-3-thien-2-yl-3H-1,4-benzodiazepin-2-amine;
N-cyclopropyl-5-phenyl-3-thien-2-yl-3H-1,4-benzodiazepin-2-amine;
N,8-dimethyl-5-phenyl-3-thien-2-yl-3H-1,4-benzodiazepin-2-amine;
N-allyl-5-phenyl-3-thien-2-yl-3H-1,4-benzodiazepin-2-amine;
5-phenyl-N-prop-2-ynyl-3-thien-2-yl-3H-1,4-benzodiazepin-2-amine;
5-phenyl-3-thien-2-yl-N-(2,2,2-trifluoroethyl)-3H-1,4-benzodiazepin-2-amine;
N,N-dimethyl-5-phenyl-3-thien-2-yl-3H-1,4-benzodiazepin-2-amine;
5-phenyl-2-pyrrolidin-1-yl-3-thien-2-yl-3H-1,4-benzodiazepine;
2-azetidin-1-yl-5-phenyl-3-thien-2-yl-3H-1,4-benzodiazepine;
2-(4,5-dihydro-1H-pyrazol-1-yl)-5-phenyl-3-thien-2-yl-3H-1,4-benzodiazepin;
2-[(5-phenyl-3-thien-2-yl-3H-1,4-benzodiazepin-2-yl)amino]ethanol;
N-(2-methoxyethyl)-5-phenyl-3-thien-2-yl-3H-1,4-benzodiazepin-2-amine;
1-(5-phenyl-3-thien-2-yl-3H-1,4-benzodiazepin-2-yl)pyrrolidin-3-ol;
1-(5-phenyl-3-thien-2-yl-3H-1,4-benzodiazepin-2-yl)azetidin-3-ol;
N-methyl-5-phenyl-3-(2-thienyl)-3H-pyrido[2,3-e][1,4]diazepin-2-amine;
N-ethyl-5-phenyl-3-thien-2-yl-3H-pyrido[2,3-e][1,4]diazepin-2-amine;
N-(2-fluoroethyl)-5-phenyl-3-thien-2-yl-3H-pyrido[2,3-e][1,4]diazepin-2-amine;
5-(4-chlorophenyl)-N-methyl-3-(2-thienyl)-3H-1,4-benzodiazepin-2-amine;
7-chloro-N-methyl-5-pyridin-3-yl-3-(2-thienyl)-3H-1,4-benzodiazepin-2-amine;
7-chloro-N-methyl-3,5-di-2-thienyl-3H-1,4-benzodiazepin-2-amine;
5-phenyl-3-(2-thienyl)-N-(2,2,2-trifluoroethyl)-3H-pyrido[2,3-e][1,4]diazepin-2-amine;
N-(2,2-difluoroethyl)-5-phenyl-3-(2-thienyl)-3H-pyrido[2,3-e][1,4]diazepin-2-amine;
1-[5-phenyl-3-(2-thienyl)-3H-pyrido[2,3-e][1,4]diazepin-2-yl]azetidin-3-ol;
1-[5-phenyl-3-(2-thienyl)-3H-pyrido[2,3-e][1,4]diazepin-2-yl]azetidin-3-amine;
N-cyclopropyl-3-(4-fluorophenyl)-5-(2-thienyl)-3H-pyrido[2,3-e][1,4]diazepin-2-amine;
N-cyclopropyl-3-(3-fluorophenyl)-5-(2-thienyl)-3H-pyrido[2,3-e][1,4]diazepin-2-amine;
N-cyclopropyl-3,5-di-2-thienyl-3H-pyrido[2,3-e][1,4]diazepin-2-amine;
N-prop-2-yn-1-yl-3,5-di-2-thienyl-3H-pyrido[2,3-e][1,4]diazepin-2-amine;
2-azetidin-1-yl-3,5-di-2-thienyl-3H-pyrido[2,3-e][1,4]diazepine;
N-ethyl-3,5-di-2-thienyl-3H-pyrido[2,3-e][1,4]diazepin-2-amine;
5-(2-furyl)-N-methyl-3-(1-methyl-1H-pyrrol-2-yl)-3H-pyrido[2,3-e][1,4]diazepin-2-amine;
N-methyl-3-(1-methyl-1H-pyrrol-2-yl)-5-(2-thienyl)-3H-pyrido[2,3-e][1,4]diazepin-2-amine;
5-(4-fluorophenyl)-N-methyl-3-thien-2-yl-3H-pyrido[2,3-e][1,4]diazepin-2-amine;
N-cyclopropyl-5-(4-fluorophenyl)-3-thien-2-yl-3H-pyrido[2,3-e][1,4]diazepin-2-amine;
N-ethyl-5-(4-fluorophenyl)-3-thien-2-yl-3H-pyrido[2,3-e][1,4]diazepin-2-amine;
5-(3-fluorophenyl)-N-methyl-3-thien-2-yl-3H-pyrido[2,3-e][1,4]diazepin-2-amine;
N-cyclopropyl-5-(3-fluorophenyl)-3-thien-2-yl-3H-pyrido[2,3-e][1,4]diazepin-2-amine;
N-ethyl-5-(3-fluorophenyl)-3-thien-2-yl-3H-pyrido[2,3-e][1,4]diazepin-2-amine;
N-methyl-5-pyridin-3-yl-3-thien-2-yl-3H-pyrido[2,3-e][1,4]diazepin-2-amine;
2-azetidin-1-yl-5-pyridin-3-yl-3-thien-2-yl-3H-pyrido[2,3-e][1,4]diazepine;
N-methyl-5-pyridin-2-yl-3-thien-2-yl-3H-pyrido[2,3-e][1,4]diazepin-2-amine;
N-methyl-3,5-di-2-thienyl-3H-1,4-benzodiazepin-2-amine;
N-methyl-3,5-di-2-thienyl-3H-pyrido[2,3-e][1,4]diazepin-2-amine;
N-methyl-5-(3-methyl-2-thienyl)-3-(2-thienyl)-3H-pyrido[2,3-e][1,4]diazepin-2-amine;
N-methyl-5-(4-methyl-1H-imidazol-5-yl)-3-(2-thienyl)-3H-pyrido[2,3-e][1,4]diazepin-2-amine;
N-methyl-5-(1,3-thiazol-2-yl)-3-(2-thienyl)-3H-pyrido[2,3-e][1,4]diazepin-2-amine;
5-(2-furyl)-N-methyl-3-(2-thienyl)-3H-pyrido[2,3-e][1,4]diazepin-2-amine;
N-methyl-5-(1,3-oxazol-2-yl)-3-thien-2-yl-3H-pyrido[2,3-e][1,4]diazepin-2-amine;
5-(1H-imidazol-4-yl)-N-methyl-3-thien-2-yl-3H-pyrido[2,3-e][1,4]diazepin-2-amine;
N-methyl-5-(1-methyl-1H-imidazol-2-yl)-3-thien-2-yl-3H-pyrido[2,3-e][1,4]diazepin-2-amine;
2-azetidin-1-yl-5-(1-methyl-1H-imidazol-2-yl)-3-thien-2-yl-3H-pyrido[2,3-e][1,4]diazepine;
N-methyl-5-(1-methyl-1H-pyrazol-3-yl)-3-thien-2-yl-3H-pyrido[2,3-e][1,4]diazepin-2-amine;
N-methyl-5-(1H-pyrrol-2-yl)-3-thien-2-yl-3H-pyrido[2,3-e][1,4]diazepin-2-amine;
N-methyl-5-(1H-pyrazol-4-yl)-3-thien-2-yl-3H-pyrido[2,3-e][1,4]diazepin-2-amine;
N-methyl-5-(1H-pyrazol-3-yl)-3-thien-2-yl-3H-pyrido[2,3-e][1,4]diazepin-2-amine;
5-(3-furyl)-N-methyl-3-thien-2-yl-3H-pyrido[2,3-e][1,4]diazepin-2-amine;
N-methyl-5-(1H-pyrrol-3-yl)-3-thien-2-yl-3H-pyrido[2,3-e][1,4]diazepin-2-amine;
2-azetidin-1-yl-5-(1H-pyrrol-3-yl)-3-thien-2-yl-3H-pyrido[2,3-e][1,4]diazepine;
N-cyclopropyl-5-(1H-pyrrol-3-yl)-3-thien-2-yl-3H-pyrido[2,3-e][1,4]diazepin-2-amine;
N-methyl-5-(1-methyl-1H-pyrazol-4-yl)-3-thien-2-yl-3H-pyrido[2,3-e][1,4]diazepin-2-amine;
N-methyl-5-(1-methyl-1H-pyrrol-3-yl)-3-thien-2-yl-3H-pyrido[2,3-e][1,4]diazepin-2-amine;
N-methyl-5-(1-methyl-1H-pyrrol-2-yl)-3-thien-2-yl-3H-pyrido[2,3-e][1,4]diazepin-2-amine;
5-isothiazol-5-yl-N-methyl-3-thien-2-yl-3H-pyrido[2,3-e][1,4]diazepin-2-amine;
N-methyl-3-thien-2-yl-5-thien-3-yl-3H-pyrido[2,3-e][1,4]diazepin-2-amine;
2-azetidin-1-yl-3-thien-2-yl-5-thien-3-yl-3H-pyrido[2,3-e][1,4]diazepine;
N-cyclopropyl-3-thien-2-yl-5-thien-3-yl-3H-pyrido[2,3-e][1,4]diazepin-2-amine;
N-methyl-3-(5-methylthien-2-yl)-5-thien-2-yl-3H-pyrido[2,3-e][1,4]diazepin-2-amine;
3-(5-chlorothien-2-yl)-N-methyl-5-thien-2-yl-3H-pyrido[2,3-e][1,4]diazepin-2-amine;
3-(5-chlorothien-2-yl)-N-cyclopropyl-5-thien-2-yl-3H-pyrido[2,3-e][1,4]diazepin-2-amine;
N-methyl-3-(3-methylthien-2-yl)-5-thien-2-yl-3H-pyrido[2,3-e][1,4]diazepin-2-amine;
N-cyclopropyl-3-(3-methylthien-2-yl)-5-thien-2-yl-3H-pyrido[2,3-e][1,4]diazepin-2-amine;
5-(2-furyl)-N-methyl-3-(3-methylthien-2-yl)-3H-pyrido[2,3-e][1,4]diazepin-2-amine;
N-methyl-3-(1,3-thiazol-2-yl)-5-thien-2-yl-3H-pyrido[2,3-e][1,4]diazepin-2-amine;
5-(2-furyl)-N-methyl-3-(1,3-thiazol-2-yl)-3H-pyrido[2,3-e][1,4]diazepin-2-amine;
3-cyclopentyl-5-(2-furyl)-N-methyl-3H-pyrido[2,3-e][1,4]diazepin-2-amine;
3-(4-chloro-2-thienyl)-N-methyl-5-(2-thienyl)-3H-pyrido[2,3-e][1,4]diazepin-2-amine;
3-(3-chloro-2-thienyl)-N-methyl-5-(2-thienyl)-3H-pyrido[2,3-e][1,4]diazepin-2-amine.

13-14. (canceled)

15. A method for the treatment of a disorder such as gastritis, mucosa-associated lymphoid tissue lymphoma, gastric adenocarcinoma, adenocarcinoma of the stomach, or ulcer diseases comprising administering to a host in need of such treatment a therapeutically effective amount of a compound as defined in claim 1.

16. A method for the treatment of infections associated with H. pylori comprising administering to a host in need of such treatment a therapeutically effective amount of a compound as defined in claim 1.

17. A method for the prophylaxis treatment of infections associated with H. pylori comprising administering to a host in need of such treatment a therapeutically effective amount of a compound as defined in claim 1.

18. A method for the treatment or prophylaxis of H. pylori infection comprising administering a therapeutically effective amount of a compound as defined in claim 1 or a pharmaceutically acceptable salt.

19. A pharmaceutical composition comprising a compound as defined in claim 1, together with at least one pharmaceutically acceptable carrier, diluent or excipient.
